SEQUENCE LISTING (149 sequences)

SEQ ID NO: 1 - protein, 8 aa, Homo sapiens
GFSFSVAW

SEQ ID NO: 2 - protein, 10 aa, Homo sapiens
IKRKTDGGTT

SEQ ID NO: 3 - protein, 20 aa, Homo sapiens
TTDPLLRYSDWLSHSLMGDV

SEQ ID NO: 4 - protein, 6 aa, Homo sapiens
SVAWMS

SEQ ID NO: 5 - protein, 19 aa, Homo sapiens
RIKRKTDGGTTDYAAPVKG

SEQ ID NO: 6 - protein, 18 aa, Homo sapiens
DPLLRYSDWLSHSLMGDV

SEQ ID NO: 7 - protein, 129 aa, Homo sapiens
EVQLVESGGGLVKPGGSLRLSCAASGFSFSVAWMSWVRQAPGKGLEWVGRIKRKTDGGTTDYAAPVKGRFIISRDDSIKALYLQMNSLKTEDTAVYYCTTDPLLRYSDWLSHSLMGDVWGQGTTVTVSS

SEQ ID NO: 8 - DNA, 388 bp, Homo sapiens
  source 1..388: mol_type = unassigned DNA
gaggtgcagctggtggagtctgggggaggcttggtaaagcctggggggtcccttagactctcctgtgcagcctctggattcagtttcagtgtcgcctggatgagctgggtccgccaggctccagggaaggggctggagtgggttggccgtattaagcgcaaaactgatggtgggacaacagactacgctgcacccgtgaaaggcagattcatcatctcaagagatgattcaataaaagcgctgtatctgcaaatgaacagcctgaaaaccgaggacacagccgtgtattactgtaccacagatcccttattacgatattctgactggttatcccactcccttatgggggacgtctggggccaagggaccacggtcaccgtctcctcag

SEQ ID NO: 9 - protein, 458 aa, Homo sapiens
EVQLVESGGGLVKPGGSLRLSCAASGFSFSVAWMSWVRQAPGKGLEWVGRIKRKTDGGTTDYAAPVKGRFIISRDDSIKALYLQMNSLKTEDTAVYYCTTDPLLRYSDWLSHSLMGDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 10 - DNA, 1375 bp, Homo sapiens
  source 1..1375: mol_type = unassigned DNA
gaggtgcagctggtggagtctgggggaggcttggtaaagcctggggggtcccttagactctcctgtgcagcctctggattcagtttcagtgtcgcctggatgagctgggtccgccaggctccagggaaggggctggagtgggttggccgtattaagcgcaaaactgatggtgggacaacagactacgctgcacccgtgaaaggcagattcatcatctcaagagatgattcaataaaagcgctgtatctgcaaatgaacagcctgaaaaccgaggacacagccgtgtattactgtaccacagatcccttattacgatattctgactggttatcccactcccttatgggggacgtctggggccaagggaccacggtcaccgtctcctcaggccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacggcgccctgcacaaccactacacccagaagtccctgtccctgagccccggc

SEQ ID NO: 11 - protein, 6 aa, Homo sapiens
QNINNY

SEQ ID NO: 12 - skipped

SEQ ID NO: 13 - protein, 13 aa, Homo sapiens
QQTYSIPLTFGGG

SEQ ID NO: 14 - protein, 5 aa, Homo sapiens
NNYLN

SEQ ID NO: 15 - protein, 12 aa, Homo sapiens
YAASSLQSGVPS

SEQ ID NO: 16 - protein, 13 aa, Homo sapiens
QQTYSIPLTFGGG

SEQ ID NO: 17 - protein, 107 aa, Homo sapiens
DIQMTQSPSSLSASVGDRVAITCRTSQNINNYLNWYQQKPGKAPKPLIYAASSLQSGVPSRFSGSGSGTDFTLTISTLHPEDFATYYCQQTYSIPLTFGGGTKVEIK

SEQ ID NO: 18 - DNA, 321 bp, Homo sapiens
  source 1..321: mol_type = unassigned DNA
gatatccagatgacccagtccccctccagcctgtctgcctctgtgggcgacagagtggccatcacctgtcggacctcccagaacatcaacaactacctgaactggtatcagcagaagcccggcaaggcccccaagcccctgatctacgctgcttccagtctgcagtccggcgtgccctctagattctccggctctggctctggcaccgacttcaccctgaccatctccaccctgcaccccgaggacttcgccacctactactgccagcagacctactccatccccctgacctttggcggaggcaccaaggtggaaatcaaa

SEQ ID NO: 19 - protein, 214 aa, Homo sapiens
DIQMTQSPSSLSASVGDRVAITCRTSQNINNYLNWYQQKPGKAPKPLIYAASSLQSGVPSRFSGSGSGTDFTLTISTLHPEDFATYYCQQTYSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 642 bp, Homo sapiens
  source 1..642: mol_type = unassigned DNA
gatatccagatgacccagtccccctccagcctgtctgcctctgtgggcgacagagtggccatcacctgtcggacctcccagaacatcaacaactacctgaactggtatcagcagaagcccggcaaggcccccaagcccctgatctacgctgcttccagtctgcagtccggcgtgccctctagattctccggctctggctctggcaccgacttcaccctgaccatctccaccctgcaccccgaggacttcgccacctactactgccagcagacctactccatccccctgacctttggcggaggcaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 21 - protein, 6 aa, Homo sapiens
QSVSSY

SEQ ID NO: 22 - skipped

SEQ ID NO: 23 - protein, 9 aa, Homo sapiens
QQRRNWPLT

SEQ ID NO: 24 - protein, 11 aa, Homo sapiens
RVSQSVSSYLA

SEQ ID NO: 25 - protein, 7 aa, Homo sapiens
DTSNRAT

SEQ ID NO: 26 - protein, 9 aa, Homo sapiens
QQRRNWPLT

SEQ ID NO: 27 - protein, 125 aa, Homo sapiens
EVQLVESGGGLVQPGGSLRLSCAASGFTFSRYAMSWVRQAPGKGLEWVSSISASGATTFYADPVKGRFTISRDNSKKTLYLQMNSLRAEDTAVYYCAKDQDFDILTGYLNWFDPWGQGTLVTVSS

SEQ ID NO: 28 - DNA, 375 bp, Homo sapiens
  source 1..375: mol_type = unassigned DNA
gaagtgcagctggttgaatctggcggcggattggttcagcctggcggatctctgagactgtcttgtgccgcctccggcttcaccttctccagatacgctatgtcctgggtccgacaggctcctggcaaaggactggaatgggtgtcctccatctctgcctctggcgccaccaccttttacgccgatcctgtgaagggcagattcaccatcagccgggacaactccaagaaaaccctgtacctgcagatgaactccctgagagccgaggacaccgccgtgtactactgtgccaaggaccaggacttcgacatcctgaccggctacctgaattggttcgacccttggggccagggcacactggttacagtgtctagc

SEQ ID NO: 29 - protein, 454 aa, Homo sapiens
EVQLVESGGGLVQPGGSLRLSCAASGFTFSRYAMSWVRQAPGKGLEWVSSISASGATTFYADPVKGRFTISRDNSKKTLYLQMNSLRAEDTAVYYCAKDQDFDILTGYLNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 30 - DNA, 1362 bp, Homo sapiens
  source 1..1362: mol_type = unassigned DNA
gaagtgcagctggttgaatctggcggcggattggttcagcctggcggatctctgagactgtcttgtgccgcctccggcttcaccttctccagatacgctatgtcctgggtccgacaggctcctggcaaaggactggaatgggtgtcctccatctctgcctctggcgccaccaccttttacgccgatcctgtgaagggcagattcaccatcagccgggacaactccaagaaaaccctgtacctgcagatgaactccctgagagccgaggacaccgccgtgtactactgtgccaaggaccaggacttcgacatcctgaccggctacctgaattggttcgacccttggggccagggcacactggttacagtgtctagcgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacggcgccctgcacaaccactacacccagaagtccctgtccctgagccccggc

SEQ ID NO: 31 - protein, 6 aa, Homo sapiens
QSVSSY

SEQ ID NO: 32 - skipped

SEQ ID NO: 33 - protein, 9 aa, Homo sapiens
QQRRNWPLT

SEQ ID NO: 34 - protein, 11 aa, Homo sapiens
RVSQSVSSYLA

SEQ ID NO: 35 - protein, 7 aa, Homo sapiens
DTSNRAT

SEQ ID NO: 36 - protein, 9 aa, Homo sapiens
QQRRNWPLT

SEQ ID NO: 37 - protein, 107 aa, Homo sapiens
EIVLTQSPATLSLSPGERVILSCRVSQSVSSYLAWYQQKPGQAPRLLIYDTSNRATGIPARFSGSGSGTDFTLTISSLEPEDFAVYYCQQRRNWPLTFGGGTKVEIK

SEQ ID NO: 38 - DNA, 321 bp, Homo sapiens
  source 1..321: mol_type = unassigned DNA
gagatcgtgctgacccagtctcctgccacactgtcactgtctccaggcgagagagtgatcctgtcttgccgggtgtcccagtccgtgtcctcttacctggcttggtatcagcagaagcccggccaggctcctagactgctgatctacgacacctccaacagagccacaggcatccccgccagattttccggctctggctctggcacagactttaccctgaccatctccagcctggaacctgaggacttcgccgtgtactactgccagcagcggagaaactggcccctgacatttggcggcggaacaaaggtggaaatcaag

SEQ ID NO: 39 - protein, 214 aa, Homo sapiens
EIVLTQSPATLSLSPGERVILSCRVSQSVSSYLAWYQQKPGQAPRLLIYDTSNRATGIPARFSGSGSGTDFTLTISSLEPEDFAVYYCQQRRNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 642 bp, Homo sapiens
  source 1..642: mol_type = unassigned DNA
gagatcgtgctgacccagtctcctgccacactgtcactgtctccaggcgagagagtgatcctgtcttgccgggtgtcccagtccgtgtcctcttacctggcttggtatcagcagaagcccggccaggctcctagactgctgatctacgacacctccaacagagccacaggcatccccgccagattttccggctctggctctggcacagactttaccctgaccatctccagcctggaacctgaggacttcgccgtgtactactgccagcagcggagaaactggcccctgacatttggcggcggaacaaaggtggaaatcaagcgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 41 - protein, 8 aa, Homo sapiens
GLTFNNYA

SEQ ID NO: 42 - protein, 8 aa, Homo sapiens
ISGSGGST

SEQ ID NO: 43 - protein, 15 aa, Homo sapiens
AKGGLLYFGEFHFDY

SEQ ID NO: 44 - protein, 4 aa, Homo sapiens
NNYA

SEQ ID NO: 45 - protein, 17 aa, Homo sapiens
TISGSGGSTYYADSAKG

SEQ ID NO: 46 - protein, 13 aa, Homo sapiens
GGLLYFGEFHFDY

SEQ ID NO: 47 - protein, 122 aa, Homo sapiens
EVQLVESGGGLVQLGGSLRLSCAASGLTFNNYAMSWVRQAPGKGLEWVSTISGSGGSTYYADSAKGRFTISRDNSKNTLYLQMNSLRAEDTAVYYCAKGGLLYFGEFHFDYWGQGTLVTVSS

SEQ ID NO: 48 - DNA, 366 bp, Homo sapiens
  source 1..366: mol_type = unassigned DNA
gaggtgcagctggtggagtctgggggaggcttggtacagcttggggggtccctgagactctcctgtgcagcctctggactcacctttaacaactatgccatgagctgggtccgccaggctccagggaaggggctggagtgggtctcaactattagtggaagtggtggcagcacatactacgcagactccgcgaagggccggttcaccatctccagagacaattccaagaacacgctgtatctgcaaatgaacagcctgagagccgaggacacggccgtatattactgtgcgaaagggggcttactatacttcggggagttccactttgactactggggccagggaaccctggtcaccgtctcctca

SEQ ID NO: 49 - protein, 451 aa, Homo sapiens
EVQLVESGGGLVQLGGSLRLSCAASGLTFNNYAMSWVRQAPGKGLEWVSTISGSGGSTYYADSAKGRFTISRDNSKNTLYLQMNSLRAEDTAVYYCAKGGLLYFGEFHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 50 - DNA, 1353 bp, Homo sapiens
  source 1..1353: mol_type = unassigned DNA
gaggtgcagctggtggagtctgggggaggcttggtacagcttggggggtccctgagactctcctgtgcagcctctggactcacctttaacaactatgccatgagctgggtccgccaggctccagggaaggggctggagtgggtctcaactattagtggaagtggtggcagcacatactacgcagactccgcgaagggccggttcaccatctccagagacaattccaagaacacgctgtatctgcaaatgaacagcctgagagccgaggacacggccgtatattactgtgcgaaagggggcttactatacttcggggagttccactttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacggcgccctgcacaaccactacacccagaagtccctgtccctgagccccggc

SEQ ID NO: 51 - protein, 6 aa, Homo sapiens
QGISNY

SEQ ID NO: 52 - skipped

SEQ ID NO: 53 - protein, 9 aa, Homo sapiens
QHYNSYPLT

SEQ ID NO: 54 - protein, 11 aa, Homo sapiens
RASQGISNYLA

SEQ ID NO: 55 - protein, 7 aa, Homo sapiens
AASSLQS

SEQ ID NO: 56 - protein, 9 aa, Homo sapiens
QHYNSYPLT

SEQ ID NO: 57 - protein, 107 aa, Homo sapiens
DIQMTQSPSSLSASVGDRVTIICRASQGISNYLAWFQQKPGKAPKSLIYAASSLQSGVPSKFSGSGSGTDFTLTISSLQPEDFATYYCQHYNSYPLTFGGGTKVEIK

SEQ ID NO: 58 - DNA, 321 bp, Homo sapiens
  source 1..321: mol_type = unassigned DNA
gacatccagatgacccagtctccatcctcactgtctgcatctgtaggagacagagtcaccatcatttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacattataatagttacccgctcactttcggcggagggaccaaggtggagatcaaa

SEQ ID NO: 59 - protein, 214 aa, Homo sapiens
DIQMTQSPSSLSASVGDRVTIICRASQGISNYLAWFQQKPGKAPKSLIYAASSLQSGVPSKFSGSGSGTDFTLTISSLQPEDFATYYCQHYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - DNA, 642 bp, Homo sapiens
  source 1..642: mol_type = unassigned DNA
gacatccagatgacccagtctccatcctcactgtctgcatctgtaggagacagagtcaccatcatttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacattataatagttacccgctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 61 - protein, 8 aa, Homo sapiens
GVTFSNAW

SEQ ID NO: 62 - protein, 10 aa, Homo sapiens
IKSKTDGGTT

SEQ ID NO: 63 - protein, 11 aa, Homo sapiens
TIEAVAGHFDY

SEQ ID NO: 64 - protein, 6 aa, Homo sapiens
SNAWMS

SEQ ID NO: 65 - protein, 19 aa, Homo sapiens
RIKSKTDGGTTDYAAPVKG

SEQ ID NO: 66 - protein, 9 aa, Homo sapiens
EAVAGHFDY

SEQ ID NO: 67 - protein, 120 aa, Homo sapiens
EVQLVESGGGLVKPGGSPRLSCAAYGVTFSNAWMSWVRQAPGKGLEWVGRIKSKTDGGTTDYAAPVKGRFTISRDDSKNTLYLQMNSLKTEDTAVYYCTIEAVAGHFDYWGQGTLVTVSS

SEQ ID NO: 68 - DNA, 360 bp, Homo sapiens
  source 1..360: mol_type = unassigned DNA
gaggtgcagttggtggagtctgggggaggcttggtaaagcctggggggtcccctagactctcctgtgcagcctatggagtcactttcagtaacgcctggatgagttgggtccgccaggctccagggaaggggctggagtgggttggccgtattaaaagcaaaactgatggtgggacaacagactatgctgcacccgtgaaaggcagattcaccatctcaagagatgattcaaaaaacacgctgtatctgcaaatgaacagcctgaaaaccgaggacacagccgtgtattactgtaccatagaagcagtggctggtcactttgactactggggccagggaaccctggtcaccgtctcctca

SEQ ID NO: 69 - protein, 449 aa, Homo sapiens
EVQLVESGGGLVKPGGSPRLSCAAYGVTFSNAWMSWVRQAPGKGLEWVGRIKSKTDGGTTDYAAPVKGRFTISRDDSKNTLYLQMNSLKTEDTAVYYCTIEAVAGHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 70 - DNA, 1347 bp, Homo sapiens
  source 1..1347: mol_type = unassigned DNA
gaggtgcagttggtggagtctgggggaggcttggtaaagcctggggggtcccctagactctcctgtgcagcctatggagtcactttcagtaacgcctggatgagttgggtccgccaggctccagggaaggggctggagtgggttggccgtattaaaagcaaaactgatggtgggacaacagactatgctgcacccgtgaaaggcagattcaccatctcaagagatgattcaaaaaacacgctgtatctgcaaatgaacagcctgaaaaccgaggacacagccgtgtattactgtaccatagaagcagtggctggtcactttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacggcgccctgcacaaccactacacccagaagtccctgtccctgagccccggc

SEQ ID NO: 71 - protein, 6 aa, Homo sapiens
QSISSW

SEQ ID NO: 72 - skipped

SEQ ID NO: 73 - protein, 9 aa, Homo sapiens
QQYNNYSPT

SEQ ID NO: 74 - protein, 11 aa, Homo sapiens
RASQSISSWLA

SEQ ID NO: 75 - protein, 7 aa, Homo sapiens
KASSLES

SEQ ID NO: 76 - protein, 9 aa, Homo sapiens
QQYNNYSPT

SEQ ID NO: 77 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNNYSPTFGQGTKVEIK

SEQ ID NO: 78 - DNA, 321 bp, Homo sapiens
  source 1..321: mol_type = unassigned DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataataattattctccgacgttcggccaggggaccaaggtggaaatcaaa

SEQ ID NO: 79 - protein, 214 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNNYSPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - DNA, 642 bp, Homo sapiens
  source 1..642: mol_type = unassigned DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataataattattctccgacgttcggccaggggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 81 - DNA, 723 bp, Homo sapiens
  source 1..723: mol_type = unassigned DNA
atggccgggcctccgaggctcctgctgctgcccctgcttctggcgctggctcgcggcctgcctggggccctggctgcccaagaggtgcagcagtctccccactgcacgactgtccccgtgggagcctccgtcaacatcacctgctccaccagcgggggcctgcgtgggatctacctgaggcagctcgggccacagccccaagacatcatttactacgaggacggggtggtgcccactacggacagacggttccggggccgcatcgacttctcagggtcccaggacaacctgactatcaccatgcaccgcctgcagctgtcggacactggcacctacacctgccaggccatcacggaggtcaatgtctacggctccggcaccctggtcctggtgacagaggaacagtcccaaggatggcacagatgctcggacgccccaccaagggcctctgccctccctgccccaccgacaggctccgccctccctgacccgcagacagcctctgccctccctgacccgccagcagcctctgccctccctgcggccctggcggtgatctccttcctcctcgggctgggcctgggggtggcgtgtgtgctggcgaggacacagataaagaaactgtgctcgtggcgggataagaattcggcggcatgtgtggtgtacgaggacatgtcgcacagccgctgcaacacgctgtcctcccccaaccagtaccagtga

SEQ ID NO: 82 - protein, 238 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQ

SEQ ID NO: 83 - DNA, 723 bp, Macaca fascicularis
  source 1..723: mol_type = unassigned DNA
atggccgggctcccgaggctcctgctgctgcccctcctcctggcgctggctcgtggcctgcctggggccctggccgcccaagaggtgcagcagtctccccactgcacgattgcccccgtgggaggctccgtcaacatcacctgctccaccagcggggaactgcatgggatctacctgaggcagctcgggccacagccccaaaacatcatttactacgaggacagggtggtgcccactacggacaaacggttccagggccgcattgacttctcggggtcccaggacaacctgactattaccatgcaccacctgcagccgtcggacactggcacctacacctgccaggccgtcacggagatcaatgtctacggctccggtaccctggtcctggtgacagaggaacagtcccaaggattgcacagatgctcggacgccccgccgacaggctctgccctccctgtcccgccgacaacctctgccctccctgccctgccaacagcctctgccctccctgccctgccgacagcctctgccctccctgtggccctggcagtggtctccttcctcctcgggctggccctgggggtggcctgtgtgctggtgaggacgcagatgaagaaactgtgctcgtcgcgggataagaattcggcagcgtgtgtggtgtacgaggacatgtcccgcagccgctgcaacacgctgtcctcccccaaccagtaccagtga

SEQ ID NO: 84 - protein, 240 aa, Macaca fascicularis
MAGLPRLLLLPLLLALARGLPGALAAQEVQQSPHCTIAPVGGSVNITCSTSGELHGIYLRQLGPQPQNIIYYEDRVVPTTDKRFQGRIDFSGSQDNLTITMHHLQPSDTGTYTCQAVTEINVYGSGTLVLVTEEQSQGLHRCSDAPPTGSALPVPPTTSALPALPTASALPALPTASALPVALAVVSFLLGLALGVACVLVRTQMKKLCSSRDKNSAACVVYEDMSRSRCNTLSSPNQYQ

SEQ ID NO: 85 - DNA, 630 bp, Rattus norvegicus
  source 1..630: mol_type = unassigned DNA
atgactcaggcactgttggctttgctacttacactggctagaatcctgcctggccccctggatgcccaagaggtacatcagtctccccgagtcgtgatcgcctccgagggagagtccatcaacatcacctgctctacaagaggggatttggaagggctcataatgaagcggatctggccacaggcttccaatgtgatttactttgaagacgaattggagcccacagtagacagtgccttctcaggccgaattaatttctctgggtcccagaagaatctgaccatcatcatgagccttctccaggaggcagacactggagcctacacctgcgaggccgtcagaaaagtcagtgtccatggcttgttcaccacggttgtggtgaaagaaaaactatcccatgaagcgtacagatcccaggaacctctgcagacatcagtttccctcccagctgccattgctgtaagcttcttcctcactgggctgatccttggggtgctgtgcagcatgctgaggaagacgcagatcaagaaactgtgtgcctcagggaataaggactctctgtgcgtcgtgtatgaagacatgtcctgcagcaaccgcaagataccatgtacccccaaccagtaccaatga

SEQ ID NO: 86 - protein, 209 aa, Rattus norvegicus
MTQALLALLLTLARILPGPLDAQEVHQSPRVVIASEGESINITCSTRGDLEGLIMKRIWPQASNVIYFEDELEPTVDSAFSGRINFSGSQKNLTIIMSLLQEADTGAYTCEAVRKVSVHGLFTTVVVKEKLSHEAYRSQEPLQTSVSLPAAIAVSFFLTGLILGVLCSMLRKTQIKKLCASGNKDSLCVVYEDMSCSNRKIPCTPNQYQ

SEQ ID NO: 87 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 88 - protein, 330 aa, Homo sapiens [sequence omitted]

SEQ ID NO: 89 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 90 - protein, 330 aa, Homo sapiens [sequence omitted]

SEQ ID NO: 91 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 92 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaa

SEQ ID NO: 94 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 95 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 96 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 978 bp, Homo sapiens
  source 1..978: mol_type = unassigned DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 98 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 99 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgacctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 100 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 101 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 102 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 103 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 104 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 105 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 106 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaa

SEQ ID NO: 108 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaa

SEQ ID NO: 110 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 111 - DNA, 981 bp, synthetic construct
  misc_feature 1..981: note = Human IgG4-PE constant region
  source 1..981: mol_type = other DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 112 - DNA, 981 bp, synthetic construct
  misc_feature 1..981: note = Human IgG4-PE constant region
  source 1..981: mol_type = other DNA
gcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 113 - DNA, 981 bp, synthetic construct
  misc_feature 1..981: note = Human IgG4-PE constant region
  source 1..981: mol_type = other DNA
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 114 - protein, 327 aa, synthetic construct
  REGION 1..327: note = Human IgG4-PE constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - DNA, 981 bp, synthetic construct
  misc_feature 1..981: note = Inactivated Human IgG4
  source 1..981: mol_type = other DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctccagttgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 116 - protein, 327 aa, synthetic construct
  REGION 1..327: note = Inactivated Human IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - DNA, 321 bp, Homo sapiens
  source 1..321: mol_type = unassigned DNA
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 118 - protein, 107 aa, Homo sapiens [sequence omitted]

SEQ ID NO: 119 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggagagcaaggacagcacctacagcctcagcagcaccctgacgctgagcaaagcagactacgagaaacacaaagtctacgccggcgaagtcacccatcagggcctgagctcgcccgtcacaaagagcttcaacaggggagagtgt

SEQ ID NO: 120 - protein, 107 aa, Homo sapiens
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 121 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagcggaaggtggataacgccctccaatcgggtaactcccaggagagtgtcacagagcaggagagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 122 - protein, 107 aa, Homo sapiens
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 123 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 124 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 125 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagctcgcccgtcacaaagagcttcaacaggggagagtgc

SEQ ID NO: 126 - protein, 107 aa, Homo sapiens
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 127 - DNA, 312 bp, Homo sapiens
  source 1..312: mol_type = unassigned DNA
cccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 128 - protein, 104 aa, Homo sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 129 - DNA, 318 bp, Homo sapiens
  source 1..318: mol_type = unassigned DNA
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 130 - DNA, 318 bp, Homo sapiens; identical to SEQ ID NO: 129
  source 1..318: mol_type = unassigned DNA

SEQ ID NO: 131 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 132 - DNA, 318 bp, Homo sapiens
  source 1..318: mol_type = unassigned DNA
ggccagcctaaggccgctccttctgtgaccctgttccccccatcctccgaggaactgcaggctaacaaggccaccctcgtgtgcctgatcagcgacttctaccctggcgccgtgaccgtggcctggaaggctgatagctctcctgtgaaggccggcgtggaaaccaccaccccttccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcc

SEQ ID NO: 133 - DNA, 318 bp, Homo sapiens
  source 1..318: mol_type = unassigned DNA
ggccagcctaaagctgcccccagcgtcaccctgtttcctccctccagcgaggagctccaggccaacaaggccaccctcgtgtgcctgatctccgacttctatcccggcgctgtgaccgtggcttggaaagccgactccagccctgtcaaagccggcgtggagaccaccacaccctccaagcagtccaacaacaagtacgccgcctccagctatctctccctgacccctgagcagtggaagtcccaccggtcctactcctgtcaggtgacccacgagggctccaccgtggaaaagaccgtcgcccccaccgagtgctcc

SEQ ID NO: 134 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 135 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 136 - protein, 106 aa, Homo sapiens; identical to SEQ ID NO: 134

SEQ ID NO: 137 - DNA, 312 bp, Homo sapiens
  source 1..312: mol_type = unassigned DNA
cccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 138 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 139 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcataagtgacttctacccggggccagtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacggaatgttca

SEQ ID NO: 140 - protein, 106 aa, Homo sapiens
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 141 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 142 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 143 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 144 - protein, 106 aa, Homo sapiens; identical to SEQ ID NO: 134

SEQ ID NO: 145 - DNA, 318 bp, Homo sapiens
  source 1..318: mol_type = unassigned DNA
ggtcagcccaaggctgccccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 146 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 147 - DNA, 318 bp, Homo sapiens
  source 1..318: mol_type = unassigned DNA
ggtcagcccaaggctgccccatcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 148 - protein, 106 aa, Homo sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 149 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcgtaagtgacttcaacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

(19) United States
(12) Patent Application Publication
(10) Pub. No.: US 2026/0098089 A1
(43) Pub. Date: Apr. 9, 2026

(54) ANTAGONIST ANTI-CD7 ANTIBODIES

(71) Applicant: KYMAB LIMITED, Cambridge (GB)

(72) Inventors: E-Chiang LEE, Cambridge (GB); Gwenoline BORHIS, Cambridge (GB); Cassandra VAN KRINKS, Cambridge (GB); Lucy HEPBURN, Cambridge (GB); Luke BAYLISS, Cambridge (GB); Miha KOSMAC, Cambridge (GB)

(21) Appl. No.: 19/322,242

(22) Filed: Sep. 8, 2025

Related U.S. Application Data
(62) Division of application No. 17/604,209, now Pat. No. 12,435,134.

(30) Foreign Application Priority Data
Apr. 18, 2019 (GB) 1905552.4

(51) Int. Cl. C07K 16/28; A61K 39/00; A61K 39/395; A61K 45/06; A61P 35/02

(52) U.S. Cl. CPC C07K 16/2803; A61K 39/39558; A61K 45/06; A61P 35/02; A61K 2039/505; C07K 2317/33; C07K 2317/34; C07K 2317/565; C07K 2317/732; C07K 2317/734

(57) ABSTRACT

The invention relates to Cluster of Differentiation 7 (CD7) antagonists, such as antibodies and fragments, as well as methods, uses and combinations.

### RELATED APPLICATIONS

This application is a divisional of U.S. patent application Ser. No. 17/604,209, filed Oct. 15, 2021, now U.S. Pat. No. 12,435,134, which is a 35 U.S.C. § 371 filing of International Patent Application No. PCT/GB2020/050976, filed Apr. 17, 2020, which claims priority to Great Britain Patent Application No. 1905552.4, filed Apr. 18, 2019, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Sep. 5, 2025, is named 769326_SA9-645USDIV_ST26.xml and is 172,617 bytes in size.

### FIELD OF THE INVENTION

The invention relates to Cluster of Differentiation 7 (CD7)-expressing cell depletion or CD7 antagonism, such as antibodies and fragments, as well as methods, uses and combinations.

### BACKGROUND

CD7 is a type I transmembrane protein expressed on the surface of T and NK lineage cells. CD7 is highly expressed in T-ALL cells. CD7 has been shown to be a validated biomarker for diagnosis of T-ALL. In addition, it is expressed in all CEBPA double mutated AML cases comprising 5-14% AML patients. It is also expressed in subset of MDS (Myelodysplastic syndromes) blasts. Based on these observations, it would be desirable to target CD7 with a ligand, such as an antibody, to kill tumour cells to treat these malignant diseases.

T-ALL is an uncommon aggressive leukaemia resulting from the malignant transformation of T cell progenitors. Rates of relapsed and refractory T-ALLs under current standard of care from multi-centre clinical trials are 20% in children and 40% in adults. Among relapsed patients, only 5% survive for more than 5 years. There is obviously high medical need for those T-ALL patients who are therapy resistant or who relapse after initial treatment.

### STATEMENT OF INVENTION

In a first configuration the invention provides:

An antibody or fragment comprising a binding site which specifically binds to CD7 (Cluster of Differentiation 7), wherein the binding site comprises a VH domain that is encoded by a nucleotide sequence that is derived from the recombination of a human VH gene segment, DH gene segment and JH gene segment, wherein the VH gene segment is selected from IGHV3-15 and IGHV3-23.

In a second configuration the invention provides:

An antibody or fragment which specifically binds to CD7 and comprises a VH domain which comprises a CDRH3 sequence of an antibody selected from G09, F05, C02 and E04; or said sequence comprising 3, 2 or 1 amino acid substitution(s).

In a third configuration the invention provides:

An antibody or fragment (optionally according to any preceding claim) which specifically binds to CD7 and comprises a VL domain which comprises a CDRL3 sequence selected from SEQ ID NO: 13, 16, 33, 36, 53, 56, 73 and 76, or said selected CDRL3 sequence comprising 3, 2 or 1 amino acid substitution(s).

In a fourth configuration the invention provides:

An antibody or fragment comprising a binding site which specifically binds to CD7, wherein the binding site comprises a VL domain that comprises the amino acid sequence of a VL domain of an antibody selected from G09, F05, C02 and E04; or an amino acid that is at least 70% identical thereto.

In a fifth configuration the invention provides:

An antibody or fragment which specifically binds to CD7 and comprises the heavy chain amino acid sequence of an antibody selected from G09, F05, C02 and E04; or an amino acid that is at least 70% identical thereto.

In a sixth configuration the invention provides:

An antibody or fragment which specifically binds to CD7 and comprises the light chain amino acid sequence of an antibody selected from G09, F05, C02 and E04; or an amino acid that is at least 70% identical thereto.

In a seventh configuration the invention provides:

An antibody or fragment which specifically binds to a human CD7 epitope that is identical to an epitope to which the antibody of any preceding claim binds.

In a eighth configuration the invention provides:

An antibody or fragment which competes for binding to human CD7 with the antibody of any preceding configuration.

In a ninth configuration the invention provides:

A combination of an amount of an anti-CD7 antibody or fragment of the invention and an amount of a chemotherapeutic agent.

In a tenth configuration the invention provides:

Use of the antibody, fragment or combination of the invention in the manufacture of a medicament for administration to a subject for treating or preventing a CD7-mediated disease or condition, optionally a cancer.

In a eleventh configuration the invention provides:

A method of treating or preventing a CD7-mediated disease or condition in a subject (optionally a cancer), the method comprising administering to said subject a therapeutically effective amount of an antibody, fragment or combination of the invention, wherein the CD7-mediated disease or condition is thereby treated or prevented.

In a twelfth configuration the invention provides:

A pharmaceutical composition comprising the antibody, fragment or combination.

In a thirteenth configuration the invention provides:

A nucleic acid that encodes a VH domain and/or a VL domain of an antibody or fragment of the invention.

In a fourteenth configuration the invention provides:

A nucleic acid that encodes a VH domain comprising the amino acid sequence of a VH domain of an antibody selected from G09, F05, C02 and E04; or an amino acid that is at least 70% identical thereto.

In a fifteenth configuration the invention provides:

A nucleic acid that encodes a VL domain comprising the amino acid sequence of a VL domain of an antibody selected from G09, F05, C02 and E04; or an amino acid that is at least 70% identical thereto.

In a sixteenth configuration the invention provides:

A nucleic acid comprising
(a) a nucleotide sequence that is at least 70% identical to the sequence of SEQ ID NO: 10; and/or
(b) a nucleotide sequence that is at least 70% identical to the sequence of SEQ ID NO: 20.

In a seventeenth configuration the invention provides:

A nucleic acid that encodes a heavy chain and/or a light chain of an antibody or fragment of the invention.

In a eighteenth configuration the invention provides:

A nucleic acid that encodes a heavy chain comprising an amino acid sequence that is at least 70% identical to SEQ ID NO: 8.

In a nineteenth configuration the invention provides:

A nucleic acid that encodes a light chain comprising an amino acid sequence that is at least 70% identical to SEQ ID NO: 18.

In a twentieth configuration the invention provides:

A nucleic acid (eg, in a host cell, eg, a CHO or HEK293 or Cos cell) comprising
(a) a nucleotide sequence that is at least 70% identical to a heavy chain sequence selected of an antibody selected from G09, F05, C02 and E04; and/or
(b) a nucleotide sequence that is at least 70% identical to a sequence selected of an antibody selected from G09, F05, C02 and E04.

In a twenty-first configuration the invention provides:

A vector comprising the nucleic acid(s); optionally wherein the vector is a CHO or HEK293 vector.

In a twenty-second configuration the invention provides:

A host cell comprising the nucleic acid(s) or the vector of the invention.

In a twenty-third configuration the invention provides:

An antibody, fragment, combination, vector, host cell, use or method as herein described.

In a twenty-fourth configuration the invention provides:

A method of diagnosing a CD7-mediated disease or condition in a subject (optionally a cancer), the method comprising combining an antibody or fragment of the invention with an isolated cell sample (eg, a blood or serum sample) and determining that cells comprised by the sample are specifically bound by the antibody or fragment.

Also provided is:

An in vitro assay for detecting CD7-positive cells in a sample, the assay comprising combining an antibody or fragment of the invention with an isolated cell sample (eg, a blood or serum sample) and determining that cells comprised by the sample are specifically bound by the antibody or fragment.

### BRIEF DESCRIPTION OF THE DRAWINGS

FIG. 1: Effect of IgG1 Fc variant on CDC activity of the anti-CD7 benchmark monoclonal antibody, RFT2. CEM cells were plated at 1,750 cells/well in a 384-well plate and human complement serum added at a 1/16 final concentration. Titrations of each RFT2 IgG1 variant as well as an isotype control were added and incubated for 2 hours at 37° C. CellTiter-Glo® was added and luminescence read on an Envision plate reader. Percentage killing was calculated for each antibody based on signal from wells without antibody and with/without cells. Error bars represent standard deviation of 3 replicates for each point. RFT2 E345R induced potent and maximum killing significantly greater than other variants. Data was analyzed using GraphPad Prism v7.02, where log antibody concentration (M) was plotted against % killing (mean±standard deviation) and a 4-parameter logistic curve fit was applied to data, allowing calculation of EC₅₀ values. The statistical comparison of the antibodies is shown in Table 6. RFT is discussed in J Immunol. 1989 Dec. 1; 143(11):3589-97, “Characterization of a human T cell-specific chimeric antibody (CD7) with human constant and mouse variable regions”, Heinrich G et al.

FIG. 2: Flow chart of antibody discovery

FIG. 3: Secondary screening of antibodies binding to CEM cells and recombinant cynomolgus CD7 CHO cells. Supernatant from hybridoma clones was collected for screening using flow cytometry for detecting the binding to CD7 expressed in CEM cells or recombinant CHO cells. The binding was represented by geomean.

FIG. 4: Characterization of mAb binding to human and cynomolgus CD7 proteins. The binding was measured by SPR. Single concentration sensorgrams of the captured mAbs interacting with Human (h) or Cynomolgus (c) CD7 proteins as indicated.

FIG. 5: Tertiary screening of mAbs in the CDC assay. The antibodies were run in duplicate for each concentration point and in 3 independent assays. The areas under the curves were used to compare the antibody potencies. 1741E04 E345R and 1741G09 E345R showed the highest potency for CEM cell killing, followed by RFT2 E345R, TH69 E345R, 1730C02 E345R, 1896A03 E345R, 1734F05 E345R, 1738B07 E345R, whereas the isotype control (IgG1 E345R) and 1734E10 E345R did not show significant efficacy in this assay. Data was analyzed using GraphPad Prism v7.02, where log antibody concentration (M) was plotted against % killing (mean±standard deviation) and a 4-parameter logistic curve fit was applied to data, allowing calculation of EC₅₀ values. The statistical comparison of the antibodies is shown in Table 6. TH69 is discussed in Br J Haematol. 1996 November; 95(2):327-38, “Therapy with CD7 monoclonal antibody TH-69 is highly effective for xenografted human T-cell ALL”, Baum W et al.

FIG. 6: Tertiary screening of mAbs in the ADCP assay. ADCP by CellTrace™ Violet (CTV)-labelled monocyte-derived macrophages of CFSE-labelled CEM cells pre-opsonised on ice with different concentrations of E345R lead antibodies (1730C02, 1734F05, 1741G09) or benchmark anti-CD7 antibody (RFT2), compared with an IgG1 isotype control. CTV-labelled macrophages were co-cultured with CFSE-labelled CEM cells for 2.5 hrs. Cells were collected, assessed for viability using a fixable amine-reactive near-infra red and analysed by flow cytometry. Tumour cell phagocytosis was detected by flow cytometry and is expressed as % phagocytosis (% phagocytosis=100×(% CFSE+/CTV+)/(% CFSE+)). The data shown are the mean±SD of duplicate samples from a representative experiment of two independent repeats.

FIGS. 7A-7B: CDC activity and in vivo pharmacokinetics of 1741G09 E345R and 1741G09 E430G. (FIG. 7A) Two antibodies, 1741G09 E345R and 1741G09 E430G, and their isotype controls were tested in the CDC assay described in the FIG. 6. Both molecules showed similar potent depleting activity (maximum killing and EC₅₀) on depletion of CEM cells. Error bars represent standard deviation of 3 replicates for each point. (FIG. 7B) Both antibodies were administrated as a single intravenous dose at 10 mg/kg into NOD SCID gamma (NSG) mice. Serum samples were taken at eight timepoints, with three mice per time point and two timepoints per mouse. The method used to quantify the concentration of antibodies was an antigen capture ELISA. Each experimental run consisted of an antibody calibration curve and two sets of QC samples run in duplicate and placed at the front and back of the plate. Antibody concentrations were calculated using SoftMax Pro7 with the standard curve using a four-parameter logistic and a 1/Y weighting. Pharmacokinetic parameters were calculated using the PKSolver Excel add-in. P value for the difference in half-life is equal to 0.0118 based on the unpaired t test. Data shown are timepoints in triplicate (from three separate mice). Data shown as the mean concentration with the standard deviation. The t_{1/2} for 1741 G09 430G is approximately 7× longer than 1741G09 345R (20.9 hrs vs 136.7 hrs) and the Cₘₐₓ is approximately 3× higher for G09 430G than G09 345R (216811 ng/mL vs 75001 ng/mL).

FIGS. 8A-8C: Potent CDC activity of 1741G09 E430G on non-relapsed and relapsed T-ALL cell lines. T-ALL cells including cell lines (FIG. 8A), patient-derived non-relapsed (FIG. 8B) and patient-derived relapsed (FIG. 8C) were used as target cells and human serum as a complement source. Titrations of 1741G09 E430G as well as an isotype control were added and incubated for 2 hours at 37° C. CellTiter-Glo® was added and luminescence read on an Envision plate reader. Percentage killing is calculated for each antibody based on signal from wells without antibody and with/without cells. Error bars represent standard deviation of 4 replicates for each point. Data was analyzed using GraphPad Prism v7.02, where log antibody concentration (M) was plotted against % killing (mean±standard deviation) and a 4-parameter logistic curve fit was applied to data, allowing calculation of EC₅₀ values.

FIG. 9: Potent ADCP activity of 1741G09 on relapsed T-ALL cell lines. Phagocytosis by CellTrace® Violet (CTV)-labelled monocyte-derived macrophages of CellTrace® CFSE-labelled CEM or patient T-ALL cells pre-opsonised with anti-CD7 antibody 1741G09 E430G or appropriate IgG1 isotype control antibody. The 1741G09 E430G anti-CD7 antibody induces concentration-dependent enhancement of CEM and paediatric patient T-ALL (PDTALL-39, -46, -47) and, to a lesser extent, adult relapsing T-ALL (PDTALL-Ad2R) phagocytosis. Cell phagocytosis of paediatric patient T-ALL using the 1714G09 E340G antibody is statistically significant (p=0.015 using paired t-test), compared to the isotype control, and achieves maximal phagocytosis of 90-95%, compared to ˜70% maximal phagocytosis for the PDTALL-Ad2R cells. Data shown are the mean±SD of samples analysed in duplicate.

FIG. 10: Cytokine release profile. The 1741G09 E430G antibody was evaluated for the ability to stimulate human PBMC's from five individual donors, as measured by the release of specific cytokines and chemokines. Corresponding isotype control (IgG1 E430G) (A) were used to monitor non-specific activation of the PBMC cultures. Super-agonistic anti-CD28 and anti-CD3 (clone OKT3) antibodies were used as positive controls, following immobilization by air-drying on tissue culture plates. Human PBMCs were seeded into the immobilized test agents in the pre-prepared plates and incubated at 37° C. for 48 hours. Following incubation, the cytokine levels from the cultures were measured by Luminex. Levels of induction of each cytokine were interpolated from a standard curve, using a 5-point non-linear regression analysis. The interpolated data were then normalized to the unstimulated control.

FIG. 11: PBMC T and NK cell depletion profile by 1741G09 E430G in the CDC assay. Human T (A) or NK (B) cells from two healthy donors were isolated from cryopreserved PBMCs using Pan Human T or NK cell isolation kits respectively (Miltenyi Biotech) and plated at 1,750 cells/well. Titrations of 1741G09 E430G as well as matched isotype control were added and incubated at 4° C. for 30 minutes to allow for antibody opsonization. Human complement serum was then added at a 1/16 final concentration and plates were incubated for 2 hours at 37° C. After incubation, CellTiter-Glo® was added to all wells and luminescence output read on an Envision plate reader. Luminescence signal correlated with the number of viable cells per well and % killing was calculated for each antibody concentration based on luminescence signal from wells without antibody (0% killing) and without cells (100% killing). Data was analyzed using GraphPad Prism v7.02, where log antibody concentration (M) was plotted against % killing (mean±standard deviation) and a 4-parameter logistic curve fit was applied to data, allowing calculation of EC₅₀ values.

FIG. 12: B cell, monocyte, NK cell and T cell survival in the human whole blood assay. Whole blood from three healthy donors (A, B, C) collected in S-Monovette® Hirudin tubes was treated with different antibodies in three independent experiments and then incubated for 20 hours at 37° C. Hirudin was used as anti-coagulant to preserve the complement activity. After incubation, immunophenotyping were analysed by flow cytometry based on the cell surface markers (T: CD45⁺CD3⁺CD19⁻; B: CD45⁺CD3⁻CD19⁺; NK: CD45⁺CD3⁻CD16⁺CD56⁺; monocyte: CD14⁺). Data sets of each donor with mean (-) are shown. NC′: negative control; IC′: isotype control; OFA: Ofatumumab; RTX: Rituximab. Means were compared by paired t-test. * refers to p-value below 0.05; ** refers to p-value below 0.01.

FIG. 13: CEM cell survival in healthy donor blood. Whole blood from three healthy donors (D0457, D0462, D0463) collected in S-Monovette® Hirudin tubes was spiked in with CellTrace® Violet (CTV)-labelled CEM cells and treated with 10 μg/mL of antibodies as indicated in three independent experiments. Hirudin was used as anti-coagulant to preserve the complement activity. After incubation for 20 hours at 37° C., immunophenotyping were analysed by flow cytometry based on the cell surface markers (T: CD45⁺CD3⁺CD19⁻; B: CD45⁺CD3⁻CD19⁺; NK: CD45⁺CD3⁻CD16⁺CD56⁺; monocyte: CD14⁺). Data sets of each donor with mean (-) are shown. IC′: isotype control; OFA: Ofatumumab. Change in cell counts, compared to the isotype control, were also determined and plotted in the right panels. Each dot represents a single donor and the mean of the 3 donors is indicated by a plain line for each condition. 50% and 90% thresholds are indicated by discontinued lines. Means were compared by paired t-test. * refers to p-value below 0.05; ** refers to p-value below 0.01; *** refers to p-value below 0.001; **** refers to p-value below 0.0001.

FIG. 14: Impact of anti-C5a Ab on 1741G09 E430G-induced cell death (n=1). CTV-labelled CEM cells were spiked in healthy donor blood with 10 μg/mL of either E430G KY1007, E430G IgG1, WT KY1007 or Ofatumumab±10 μg/mL of anti-C5a Ab and incubated for 20 hours at 37° C. Similar methods to the previous section were used in this assay.

FIG. 15: 1741G09 E430G prolonged the survival of xenograft mice. The NSG mice were injected with 5×10⁶ T-ALL cells, PDTALL46 at day 0 and then dosed at 10 mg/Kg three times a week from day 3 until the end of the study. Two groups (10 mice/group) were treated with 1741G09 E430G and the corresponding isotype control respectively. (A) Kaplan Meier plot showing animals left on study in these two groups. ***: p<0.0001 Log-Rank (Mantel-Cox) Test compared to isotype control. (B) Individual animal flow cytometry data showing the percentage of cells expressing human CD5 on the cell surface present in the blood of the mice on study.

FIG. 16: Complement activity during chemotherapy (see ref 15).

FIG. 17: CD7 and CRP expression profiles of relapsed T-ALL cell lines, CEM and peripheral T and NK cells

FIG. 18: Pathways of complement activation

FIG. 19: CD7 and CRP expression profiles of non-relapsed T-ALL cell lines

FIG. 20: CD7 and CRP expression profiles of relapsed T-ALL cell lines

### DETAILED DESCRIPTION

### Definitions

Unless otherwise defined herein, scientific and technical terms shall have the meanings that are commonly understood by those of ordinary skill in the art. Further, unless otherwise required by context, singular terms shall include pluralities and plural terms shall include the singular.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.” In the specification and claims, the term “about” is used to modify, for example, the quantity of an ingredient in a composition, concentration, volume, process temperature, process time, yield, flow rate, pressure, and like values, and ranges thereof, employed in describing the embodiments of the disclosure. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

As used herein, “administer” or “administration” refers to the act of injecting or otherwise physically delivering a substance as it exists outside the body (e.g., an anti-hCD7 antibody provided herein) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

The term “antibody”, “immunoglobulin” or “Ig” may be used interchangeably herein and means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies (including dual binding antibodies), chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. The term “antibody” can also refer to a Y-shaped glycoprotein with a molecular weight of approximately 150 kDa that is made up of four polypeptide chains: two light (L) chains and two heavy (H) chains. There are five types of mammalian Ig heavy chain isotypes denoted by the Greek letters alpha (α), delta (δ), epsilon (ε), gamma (γ), and mu (μ). The type of heavy chain defines the class of antibody, i.e., IgA, IgD, IgE, IgG, and IgM, respectively. The γ and α classes are further divided into subclasses on the basis of differences in the constant domain sequence and function, e.g., IgG1, hIgG2, mIgG2A, mIgG2B, IgG3, IgG4, IgA1 and IgA2. In mammals, there are two types of immunoglobulin light chains, λ and κ. The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. An example of antibodies are heavy chain-only (ie, H2) antibodies that comprise a dimer of a heavy chain (5′-VH-(optional Hinge)-CH2-CH3-3′) and are devoid of a light chain.

The antibodies described herein may be oligoclonal, polyclonal, monoclonal (including full-length monoclonal antibodies), camelised, chimeric, CDR-grafted, multi-specific, bi-specific (including dual-binding antibodies), catalytic, chimeric, humanized, fully human, anti-idiotypic, including antibodies that can be labelled in soluble or bound form as well as fragments, variants or derivatives thereof, either alone or in combination with other amino acid sequences provided by known techniques. An antibody may be from any species. Antibodies described herein can be naked or conjugated to other molecules such as toxins, radioisotopes, etc.

The term “antigen binding site,” “antigen binding domain,” “antigen binding region,” “antigen binding fragment,” and similar terms refer to that portion of an antibody which comprises the amino acid residues that interact with an antigen and confer on the binding agent its specificity and affinity for the antigen (e.g. the complementarity determining regions (CDRs)). The antigen binding region can be derived from any animal species, such as rodents (e.g. rabbit, rat or hamster) and humans. Preferably, the antigen binding region will be of human origin.

Antigen binding fragments described herein can include single-chain Fvs (scFv), single-chain antibodies, single domain antibodies, domain antibodies, Fv fragments, Fab fragments, F(ab′) fragments, F(ab′)₂ fragments, antibody fragments that exhibit the desired biological activity, disulfide-stabilised variable region (dsFv), dimeric variable region (diabody), anti-idiotypic (anti-Id) antibodies (including, e.g. anti-Id antibodies to antibodies), intrabodies, linear antibodies, single-chain antibody molecules and multispecific antibodies formed from antibody fragments and epitope-binding fragments of any of the above. In particular, antibodies and antibody fragments described herein can include immunoglobulin molecules and immunologically active fragments of immunoglobulin molecules, i.e., molecules that contain an antigen-binding site. Digestion of antibodies with the enzyme, papain, results in two identical antigen-binding fragments, known also as “Fab” fragments, and a “Fc” fragment, having no antigen-binding activity but having the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulfides. The effector functions of antibodies are determined by sequences in the Fc region, the region which is also recognized by Fc receptors (FcR) found on certain types of cells. Digestion of antibodies with the enzyme, pepsin, results in a F(ab′)₂ fragment in which the two arms of the antibody molecule remain linked and comprise two-antigen binding sites. The F(ab′)₂ fragment has the ability to crosslink antigen.

The term “derived from the recombination of” in relation to gene segments will be readily apparent to the skilled person, who will understand that B-cells recombine their variable region gene segments to produce coding sequence for variable domains. For example “derived from the recombination of a human VH gene segment, DH gene segment and JH gene segment” relates to the recombination of one human VH gene segment, with one DH gene segment and one JH gene segment together to form a rearranged VDJ sequence encoding a heavy chain antibody variable domain. Junctional and somatic hypermutation may also be features of the process, whereby the resulting recombined VDJ sequence includes one or more nucleotide additions, substitutions or deletions (eg, p-additions and/or n-additions) that are not comprised by the germline V, D and J sequences. The equivalent will be said of VK and JK gene segments for a kappa light chain variable domain, and of Vλ and Jλ for a lambda light chain variable domain. It is intended that any post-translational modifications may additionally encompassed in variable domains.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “monoclonal antibody” as used herein refers to an antibody obtained from a population of substantially homogeneous antibodies, i.e. the individual antibodies comprising the population are identical except for possible naturally occurring mutations and/or post-translation modifications (e.g. isomerizations, amidations) that may be present in minor amounts. Monoclonal antibodies are highly specific and are directed against a single antigenic determinant or epitope. In contrast, polyclonal antibody preparations typically include different antibodies directed against different antigenic determinants (or epitopes). The term “monoclonal antibody” as used herein encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, hybridoma, phage selection, recombinant expression, and transgenic animals.

The monoclonal antibodies herein can include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies that exhibit the desired biological activity.

The term “humanised antibody” refers to a subset of chimeric antibodies in which a “hypervariable region” from a non-human immunoglobulin (the donor antibody) replaces residues from a hypervariable region in a human immunoglobulin (recipient antibody). In general, a humanized antibody will include substantially all of at least one, and typically two, variable domains, in which all or substantially all of the hypervariable loops correspond to those of a non-human immunoglobulin sequence, and all or substantially all of the framework regions are those of a human immunoglobulin sequence, although the framework regions may include one or more substitutions that improve antibody performance, such as binding affinity, isomerization, immunogenicity, etc.

Where utilised: ScFv is single variable fragment; Fc is fragment crystallisable; Fab is fragment antigen-binding, mAB is monoclonal antibody, dAb is domain antibody; sc is single chain; KIH is knobs-into-holes; TCR is T cell receptor; DT is dual targeting; IgG is Immunoglobulin G; Fv is variable fragment

The term “bispecific antibody” means an antibody which comprises specificity for two target molecules, and includes, but is not limited to, formats such as Dual-Variable-Domain Immunoglobulins (DVD-Ig™) (see DiGiammarino et al., “Design and generation of DVD-Ig™ molecules for dual-specific targeting”, Meth. Mo. Biol., 2012, 889, 145-156), mAb² (see WO2008/003103, the description of the mAb² format is incorporated herein by reference), FIT-Ig® (see WO2015/103072, the description of the Fabs-In-Tandem Immunoglobulin (FIT-Ig®) scaffold is incorporated herein by reference), mAb-dAb, dock and lock, Fab-arm exchange, strand-exchange engineered domain body (SEEDbody), Trifunctional antibodies (Triomab), LUZ-Y, Fc domain with antigen-binding sites (Fcab), κλ-body, orthogonal Fab, scDiabody-Fc, diabody-Fc, tandem scFv-Fc, Fab-scFv-Fc, Fab-scFv, intrabody, Bi-specific T-cell engager (BiTE), diabody, dual affinity retargeting antibody (DART), Tandem Diabody (TandAb®), scDiabody, scDiabody-CH3, Diabody-CH3, Triple body, Miniantibody, minibody, trimeric bispecific minibody (TriBi minibody), scFv-CH3 KIH, scFv-CH-CL-scFv, F(ab′)2-scFv, scFv-KIH, Fab-scFv-Fc, tetravalent heavy chain-only antibodies (HCab), Immune-mobilizing monoclonal TCRs Against Cancer (ImmTAC), knobs-in-holes, knobs-in-holes with common light chain, knobs-in-holes with common light chain and charge pairs, charge pairs, charge pairs with common light chain, DT-IgG, DutaMab™, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and Zybody™. For a review of bispecific formats, see Spiess, C., et al., Mol. Immunol. (2015). In another embodiment, the bispecific molecule comprises an antibody which is fused to another non-Ig format, for example a T-cell receptor binding domain; an immunoglobulin superfamily domain; an agnathan variable lymphocyte receptor; a fibronectin domain (e.g. an Adnectin™); an antibody constant domain (e.g. a CH₃ domain, e.g., a CH₂ and/or CH₃ of an Fcab™) wherein the constant domain is not a functional CH₁ domain; an scFv; an (scFv)₂; an sc-diabody; an scFab; a centyrin and an epitope binding domain derived from a scaffold selected from CTLA-4 (Evibody™); a lipocalin domain; Protein A such as Z-domain of Protein A (e.g. an Affibody™ or SpA); an A-domain (e.g. an Avimer™ or Maxibody™); a heat shock protein (such as and epitope binding domain derived from GroEI and GroES); a transferrin domain (e.g. a trans-body); ankyrin repeat protein (e.g. a DARPin™); peptide aptamer; C-type lectin domain (e.g. Tetranectin™); human γ-crystallin or human ubiquitin (an affilin); a PDZ domain; scorpion toxin; and a kunitz type domain of a human protease inhibitor.

In one embodiment, the bispecific antibody is a mAb². A mAb² comprises a V_{H} and V_{L} domain from an intact antibody, fused to a modified constant region, which has been engineered to form an antigen-binding site, known as an “Fcab”. The technology behind the Fcab/mAb² format is described in more detail in WO2008/003103, and the description of the mAb² format is incorporated herein by reference.

In another embodiment, the bispecific antibody is a “dual binding antibody”. As used herein, the term “dual binding antibody” is a bispecific antibody wherein both antigen-binding domains are formed by a V_{H}/V_{L} pair, and includes FIT-Ig® (see WO2015/103072, incorporated herein by reference), mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb®, scDiabody, scDiabody-CH3, Diabody-CH3, Triple body, Miniantibody, minibody, scFv-CH₃ KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab™ IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv and scFv4-Ig.

The term “hypervariable region”, “CDR region” or “CDR” refers to the regions of an antibody variable domain which are hypervariable in sequence and/or form structurally defined loops. Generally, antigen binding sites of an antibody include six hypervariable regions: three in the V_{H} (CDRH1, CDRH2, CDRH3), and three in the V_{L} (CDRL1, CDRL2, CDRL3). These regions of the heavy and light chains of an antibody confer antigen-binding specificity to the antibody. CDRs may be defined according to the Kabat system (see Kabat, E. A. et al., 1991, “Sequences of Proteins of Immunological Interest”, 5^{th} edit., NIH Publication no. 91-3242, U.S. Department of Health and Human Services). Other systems may be used to define CDRs, which as the system devised by Chothia et al (see Chothia, C. & Lesk, A. M., 1987, “Canonical structures for the hypervariable regions of immunoglobulins”, J. Mol. Biol., 196, 901-917) and the IMGT system (see Lefranc, M. P., 1997, “Unique database numbering system for immunogenetic analysis”, Immunol. Today, 18, 50). An antibody typically contains 3 heavy chain CDRs and 3 light chain CDRs. The term CDR or CDRs is used here to indicate one or several of these regions. A person skilled in the art is able to readily compare the different systems of nomenclature and determine whether a particular sequence may be defined as a CDR.

A “human antibody” is an antibody that possesses an amino-acid sequence corresponding to that of an antibody produced by a human and/or has been made using any of the techniques for making human antibodies and specifically excludes a humanized antibody comprising non-human antigen-binding residues. The term “specifically binds to” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g. by a radioimmunoassay (RIA).

An antibody or a fragment thereof that specifically binds to a human CD7 (hCD7) antigen may be cross-reactive with related antigens. Preferably, an antibody or a fragment thereof that specifically binds to a hCD7 antigen does not cross-react with other antigens (but may optionally cross-react with CD7 of a different species, e.g. rhesus, or murine). An antibody or a fragment thereof that specifically binds to a hCD7 antigen can be identified, for example, by immunoassays, BIAcore™, or other techniques known to those of skill in the art. An antibody or a fragment thereof binds specifically to a CD7 antigen when it binds to a hCD7 antigen with higher affinity than to any cross-reactive antigen as determined using experimental techniques, such as radioimmunoassays (RIA) and enzyme-linked immunosorbent assays (ELISAs). Typically, a specific or selective reaction will be at least twice background signal or noise and more typically more than 10 times (such as more than 15 times, more than 20 times, more than 50 times or more than 100 times) background. See, e.g. Paul, ed., 1989, Fundamental Immunology Second Edition, Raven Press, New York at pages 332-336 for a discussion regarding antibody specificity.

The term “aliphatic amino acid” means that the amino acid R groups are nonpolar and hydrophobic. Hydrophobicity increases with increasing number of C atoms in the hydrocarbon chain. Glycine, Alanine, Valine, Leucine and Isoleucine are aliphatic amino acids.

The term “aromatic amino acid” means that the amino acid R groups contain an aromatic ring system. Phenylalanine, Tyrosine and Tryptophan are aromatic amino acids.

The term “hydroxyl-containing amino acid” means that the amino acid R groups contain a hydroxyl group and are hydrophilic. Serine, Cysteine, Threonine and Methionine are hydroxyl-containing amino acids.

The term “basic amino acid” means that the amino acid R groups are nitrogen containing and are basic at neutral pH. Histidine, Lysine and Arginine are basic amino acids.

The term “cyclic amino acid” means that the amino acid R groups have an aliphatic cyclic structure. Proline is the only cyclic aliphatic amino acid.

The term “acidic amino acid” means that the amino acid R groups are polar and are negatively charged at physiological pH. Aspartate and Glutamate are acidic amino acids.

The term “amide amino acid” means that the amino acid R groups contain an amide group. Asparagine and Glutamine are amide amino acids.

As used herein, “authorization number” or “marketing authorization number” refers to a number issued by a regulatory agency upon that agency determining that a particular medical product and/or composition may be marketed and/or offered for sale in the area under the agency's jurisdiction. As used herein “regulatory agency” refers to one of the agencies responsible for evaluating, e.g. the safety and efficacy of a medical product and/or composition and controlling the sales/marketing of such products and/or compositions in a given area. The Food and Drug Administration (FDA) in the US and the European Medicines Agency (EPA) in Europe are but two examples of such regulatory agencies. Other non-limiting examples can include SDA, MPA, MHPRA, IMA, ANMAT, Hong Kong Department of Health-Drug Office, CDSCO, Medsafe, and KFDA.

As used herein, a “buffer” refers to a chemical agent that is able to absorb a certain quantity of acid or base without undergoing a strong variation in pH.

As used herein, the term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

The term “chemotherapeutic agent” or “chemotherapy” refers to a therapeutic agent whose primary purpose is to destroy cancer cells, typically by interfering with the tumour cell's ability to grow or multiply. There are many different types of chemotherapeutic agents, with more than 50 approved chemotherapy drugs available. Chemotherapeutic drugs can be classified based on how they work. Alkylating drugs kill cancer cells by directly attacking DNA, the genetic material of the genes. Cyclophosphamide is an alkylating drug. Antimetabolites interfere with the production of DNA and keep cells from growing and multiplying. An example of an antimetabolite is 5-fluorouracil (5-FU). Anti-tumour antibiotics are made from natural substances such as fungi in the soil. They interfere with important cell functions, including production of DNA and cell proteins. Doxorubicin and bleomycin belong to this group of chemotherapy drugs. Plant alkaloids prevent cells from dividing normally. Vinblastine and vincristine are plant alkaloids obtained from the periwinkle plant. Steroid hormones slow the growth of some cancers that depend on hormones. For example, tamoxifen is used to treat breast cancers that depend on the hormone estrogen for growth. DNA damage response (DDR) inhibitors, such as PARP inhibitors, block DNA repair mechanisms following single or double stranded breaks.

Examples of chemotherapeutic agents include Adriamycin, Doxorubicin, 5-Fluorouracil, Cytosine arabinoside (Ara-C), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Carminomycin, Aminopterin, Dactinomycin, Mitomycins, Esperamicins (see, U.S. Pat. No. 4,675,187), Melphalan, and other related nitrogen mustards. Suitable toxins and chemotherapeutic agents are described in Remington's Pharmaceutical Sciences, 19th Ed. (Mack Publishing Co. 1995), and in Goodman and Gilman's The Pharmacological Basis of Therapeutics, 7^{th} Ed. (MacMillan Publishing Co. 1985). Another example of chemotherapeutic agents is the class of antibody-conjugated toxins, including, but not limited to pyrrolobenzodiazepiness, maytansanoids, calicheamicin, etc. Other suitable toxins and/or chemotherapeutic agents are known to those of skill in the art.

As used herein, the term “composition” is intended to encompass a product containing the specified ingredients (e.g. an antibody of the invention) in, optionally, the specified amounts, as well as any product which results, directly or indirectly, from combination of the specified ingredients in, optionally, the specified amounts.

As used herein the term “comprising” or “comprises” is used with reference to antibodies, fragments, uses, compositions, methods, and respective component(s) thereof, that are essential to the method or composition, yet open to the inclusion of unspecified elements, whether essential or not.

The term “consisting of” refers to antibodies, fragments, uses, compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

In the context of a polypeptide, the term “derivative” as used herein includes a polypeptide that comprises an amino acid sequence of a hCD7 polypeptide, a fragment of a hCD7 polypeptide, or an antibody or fragment that specifically binds to a hCD7 polypeptide which has been altered by the introduction of amino acid residue substitutions, deletions or additions. The term “derivative” as used herein also includes a hCD7 polypeptide, a fragment of a hCD7 polypeptide, or an antibody that specifically binds to a hCD7 polypeptide which has been chemically modified, e.g. by the covalent attachment of any type of molecule to the polypeptide. For example, but not by way of limitation, a hCD7 polypeptide, a fragment of a hCD7 polypeptide, or a hCD7 antibody may be chemically modified, e.g. by glycosylation, acetylation, pegylation, phosphorylation, amidation, derivatization by known protecting/blocking groups, proteolytic cleavage, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. A derivative of a hCD7 polypeptide, a fragment of a hCD7 polypeptide, or a hCD7 antibody may be chemically modified by chemical modifications using techniques known to those of skill in the art, including, but not limited to specific chemical cleavage, acetylation, formulation, metabolic synthesis of tunicamycin, etc. Further, a derivative of a hCD7 polypeptide, a fragment of a hCD7 polypeptide, or a hCD7 antibody may contain one or more non-classical amino acids. A polypeptide derivative possesses a similar or identical function as a hCD7 polypeptide, a fragment of a hCD7 polypeptide, or a hCD7 antibody described herein.

The term “effector function” (or “effector-enabled”) as used herein refers to one or more of antibody dependent cell mediated cytotoxic activity (ADCC), complement-dependent cytotoxic activity (CDC) mediated responses, Fc-mediated phagocytosis, antibody dependent cellular phagocytosis (ADCP) or antibody-dependent trogocytosis and antibody recycling via the FcRn receptor.

An “effective amount” refers to an amount effective, at dosages and for periods of time necessary, to achieve the desired effect, including a therapeutic or prophylactic result. A “therapeutically effective amount” refers to the minimum concentration required to effect a measurable improvement or prevention of a particular disorder. A therapeutically effective amount herein may vary according to factors such as the disease state, age, sex, and weight of the patient, and the ability of the antibody to elicit a desired response in the individual. A therapeutically effective amount is also one in which toxic or detrimental effects of the antibody are outweighed by the therapeutically beneficial effects. A “prophylactically effective amount” refers to an amount effective, at the dosages and for periods of time necessary, to achieve the desired prophylactic result. In some embodiments, the effective amount of an antibody of the invention is from about 0.1 mg/kg (mg of antibody per kg weight of the subject) to about 100 mg/kg. In certain embodiments, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein). In some embodiments, “effective amount” as used herein also refers to the amount of an antibody of the invention to achieve a specified result (e.g. inhibition of a hCD7 biological activity of a cell). The term “epitope” as used herein refers to a localized region on the surface of an antigen, such as hCD7 polypeptide or hCD7 polypeptide fragment, that is capable of being bound to one or more antigen binding regions of an antibody, and that has antigenic or immunogenic activity in an animal, preferably a mammal, and most preferably in a human, that is capable of eliciting an immune response. An epitope having immunogenic activity is a portion of a polypeptide that elicits an antibody response in an animal. An epitope having antigenic activity is a portion of a polypeptide to which an antibody specifically binds as determined by any method well known in the art, for example, by the immunoassays described herein. Antigenic epitopes need not necessarily be immunogenic. Epitopes usually consist of chemically active surface groupings of molecules such as amino acids or sugar side chains and have specific three-dimensional structural characteristics as well as specific charge characteristics. A region of a polypeptide contributing to an epitope may be contiguous amino acids of the polypeptide or the epitope may come together from two or more non-contiguous regions of the polypeptide. The epitope may or may not be a three-dimensional surface feature of the antigen. In certain embodiments, a hCD7 epitope is a three-dimensional surface feature of a hCD7 polypeptide (e.g. in a trimeric form of a hCD7 polypeptide). In other embodiments, a hCD7 epitope is linear feature of a hCD7 polypeptide (e.g. in a trimeric form or monomeric form of the hCD7 polypeptide). Antibodies provided herein may specifically bind to an epitope of the monomeric (denatured) form of hCD7, an epitope of the trimeric (native) form of hCD7, or both the monomeric (denatured) form and the trimeric (native) form of hCD7. In specific embodiments, the antibodies provided herein specifically bind to an epitope of the trimeric form of hCD7 but do not specifically bind the monomeric form of hCD7.

The term “excipients” as used herein refers to inert substances which are commonly used as a diluent, vehicle, preservatives, binders, or stabilizing agent for drugs and includes, but not limited to, proteins (e.g. serum albumin, etc.), amino acids (e.g. aspartic acid, glutamic acid, lysine, arginine, glycine, histidine, etc.), fatty acids and phospholipids (e.g. alkyl sulfonates, caprylate, etc.), surfactants (e.g. SDS, polysorbate, nonionic surfactant, etc.), saccharides (e.g. sucrose, maltose, trehalose, etc.) and polyols (e.g. mannitol, sorbitol, etc.). See, also, Remington's Pharmaceutical Sciences (1990) Mack Publishing Co., Easton, Pa., which is hereby incorporated by reference in its entirety.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, CD7 fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a hCD7 polypeptide or an antibody that specifically binds to a hCD7 polypeptide. In a specific embodiment, a fragment of a hCD7 polypeptide or an antibody that specifically binds to a hCD7 antigen retains at least 1, at least 2, or at least 3 functions of the polypeptide or antibody.

The term “free” can refer to a polypeptide, for example, CD7 or fragments and variants thereof, that is combined with a buffer, wherein the polypeptide is not associated with a cell surface or cell membrane. As such, the term “free” can refer to a polypeptide that is capable of surface expression (i.e. includes one or more transmembrane domains or membrane association domains), but that is not, in its present state, expressed on the surface of a cell or bound to a protein that is expressed on the surface of a cell. A free polypeptide can also refer to a free recombinant or native or unbound polypeptide. In the context of phage display, a free antigen can be selected in solution (referred to herein as a “soluble selection”) or adsorbed to a surface, for example, adsorbed to the surface of a 96-well plate (referred to herein as “biopanning selection”).

The term “fusion protein” as used herein refers to a polypeptide that comprises an amino acid sequence of an antibody and an amino acid sequence of a heterologous polypeptide or protein (i.e. a polypeptide or protein not normally a part of the antibody (e.g. a non-anti-CD7 antigen antibody)). The term “fusion” when used in relation to CD7 or to an anti-CD7 antibody refers to the joining of a peptide or polypeptide, or fragment, variant and/or derivative thereof, with a heterologous peptide or polypeptide. Preferably, the fusion protein retains the biological activity of the CD7 or anti-CD7 antibody. In certain embodiments, the fusion protein comprises a CD7 antibody VH domain, V_{L} domain, VH CDR (one, two or three VH CDRs), and/or V_{L} CDR (one, two or three V_{L} CDRs), wherein the fusion protein specifically binds to a CD7 epitope.

The term “heavy chain” when used with reference to an antibody refers to five distinct types, called alpha (α), delta (δ), epsilon (ε), gamma (γ) and mu (μ), based on the amino acid sequence of the heavy chain constant domain. These distinct types of heavy chains are well known and give rise to five classes of antibodies, IgA, IgD, IgE, IgG and IgM, respectively, including four subclasses of IgG, namely IgG1, IgG2, IgG3 and IgG4. Preferably the heavy chain is a human heavy chain. In the human population, multiple heavy chain constant region alleles, of each immunoglobulin or immunoglobulin subclass, exist. The nucleotide and amino acid sequences of these allelic variants are accessible on publicly available databases such as IMGT, ENSEMBL Swiss-Prot and Uniprot. Allelic variants may also be identified in various genome sequencing projects. In one embodiment, the antibodies and antibody fragments disclosed herein comprise a heavy chain encoded by a IgG1 constant region allele, which includes, but is not limited to, human IGHG1*01, IGHG1*02, IGHG1*03, IGHG1*04 and IGHG1*05. In one embodiment, the antibodies and antibody fragments disclosed herein comprise a protein encoded by a IgG2 constant region allele, which includes, but is not limited to, human IGHG2*01, IGHG2*02, IGHG2*03, IGHG2*04, IGHG2*05 and IGHG2*06. In one embodiment, the antibodies or antibody fragments disclosed herein comprise a protein encoded by a IgG3 constant region allele, which includes but is not limited to human IGHG3*01, IGHG3*02, IGHG3*03, IGHG3*04, IGHG3*05, IGHG3*06, IGHG3*07, IGHG3*08, IGHG3*09, IGHG3*10, IGHG3*11, IGHG3*12, IGHG3*13, IGHG3*14, IGHG3*15, IGHG3*16, IGHG3*17, IGHG3*18 and IGHG3*19. In one embodiment, the antibodies or antibody fragments disclosed herein comprise a protein encoded by a IgG4 constant region allele, which includes but is not limited to human IGHG4*01 (see, eg, the sequence table herein), IGHG4*02 (see, eg, the sequence table herein), IGHG4*03 (see, eg, the sequence table herein) and IGHG4*04 (see, eg, the sequence table herein).

In another example, the heavy chain is a disabled IgG isotype, e.g. a disabled IgG4. In certain embodiments, the antibodies of the invention comprise a human gamma 4 constant region. In another embodiment, the heavy chain constant region does not bind Fc-γ receptors, and e.g. comprises a Leu235Glu mutation. In another embodiment, the heavy chain constant region comprises a Ser228Pro mutation to increase stability. In another embodiment, the heavy chain constant region is IgG4-PE (see, eg, the sequence table herein). In another embodiment, the antibodies and antibody fragments disclosed herein comprise a heavy chain constant region encoded by a murine IgG1 constant region allele, which includes but is not limited to mouse IGHG1*01 or IGHG1*02. In one embodiment, the antibodies and antibody fragments disclosed herein comprise a heavy chain constant region encoded by a murine IgG2 constant region allele, which includes, but is not limited to, mouse IGHG2A*01, IGHG2A*02, IGHG2B*01, IGHG2B*02, IGHG2C*01, IGHG2C*02 or IGHG2C*03. In one embodiment, the antibodies or antibody fragments disclosed herein comprise a protein encoded by a murine IgG3 constant region allele, which includes but is not limited to mouse IGHG3*01.

The term “host” as used herein refers to an animal, preferably a mammal, and most preferably a human.

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “in combination” in the context of the administration of other therapies refers to the use of more than one therapy. The use of the term “in combination” does not restrict the order in which therapies are administered to a subject with a disease. A first therapy can be administered before (e.g. 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g. 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a CD7-mediated disease. Any additional therapy can be administered in any order with the other additional therapies. In certain embodiments, the antibodies of the invention can be administered in combination with one or more therapies (e.g. therapies that are not the antibodies of the invention that are currently administered to prevent, treat, manage, and/or ameliorate a CD7-mediated disease. Non-limiting examples of therapies that can be administered in combination with an antibody of the invention include analgesic agents, anaesthetic agents, antibiotics, or immunomodulatory agents or any other agent listed in the U.S. Pharmacopoeia and/or Physician's Desk Reference.

As used herein, “injection device” refers to a device that is designed for carrying out injections, an injection including the steps of temporarily fluidically coupling the injection device to a person's tissue, typically the subcutaneous tissue. An injection further includes administering an amount of liquid drug into the tissue and decoupling or removing the injection device from the tissue. In some embodiments, an injection device can be an intravenous device or IV device, which is a type of injection device used when the target tissue is the blood within the circulatory system, e.g. the blood in a vein. A common, but non-limiting example of an injection device is a needle and syringe.

As used herein, “instructions” refers to a display of written, printed or graphic matter on the immediate container of an article, for example the written material displayed on a vial containing a pharmaceutically active agent, or details on the composition and use of a product of interest included in a kit containing a composition of interest. Instructions set forth the method of the treatment as contemplated to be administered or performed.

An “isolated” or “purified” antibody or protein is one that has been identified, separated and/or recovered from a component of its production environment (e.g. natural or recombinant). For example, the antibody or protein is substantially free of cellular material or other contaminating proteins from the cell or tissue source from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e. culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals which are involved in the synthesis of the protein. Accordingly, such preparations of the antibody have less than about 30%, 20%, 10%, 5% (by dry weight) of chemical precursors or compounds other than the antibody of interest. In a preferred embodiment, antibodies of the invention are isolated or purified.

The terms “Kabat numbering,” and like terms are recognized in the art and refer to a system of numbering amino acid residues which are more variable (i.e. hypervariable) than other amino acid residues in the heavy chain variable regions of an antibody, or an antigen binding portion thereof (Kabat et al., (1971) Ann. NY Acad. Sci., 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region typically ranges from amino acid positions 31 to 35 for CDR1, amino acid positions 50 to 65 for CDR2, and amino acid positions 95 to 102 for CDR3.

“Label” or “labelled” as used herein refers to the addition of a detectable moiety to a polypeptide, for example, a radiolabel, fluorescent label, enzymatic label, chemiluminescent label or a biotinyl group or gold. Radioisotopes or radionuclides may include ³H, ⁴C, ¹⁵N, ³⁵S, ⁹⁰Y, ⁹⁹Tc, ¹¹⁵In, ¹²⁵I, ¹³¹I, fluorescent labels may include rhodamine, lanthanide phosphors or FITC and enzymatic labels may include horseradish peroxidase, 3-galactosidase, luciferase, alkaline phosphatase. Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes (e.g. cyanine dyes, e.g. Cy5™, Cy5.5™, or Cy7™); additional fluorescent labels or fluorescers include, such as fluorescein and its derivatives, fluorochrome, GFP (GFP for “Green Fluorescent Protein”), other fluorescent proteins (e.g. mCherry, mTomato), dansyl, umbelliferone, phycoerythrin, phycocyanin, allophycocyanin, o-phthaldehyde, and fluorescamine; fluorophores such as lanthanide cryptates and chelates e.g. Europium etc (Perkin Elmer and Cisbio Assays); chemoluminescent labels or chemiluminescers, such as isoluminol, luminol and the dioxetanes; sensitisers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The term “light chain” when used in reference to an antibody refers to the immunoglobulin light chains, of which there are two types in mammals, lambda (A) and kappa (κ). Preferably, the light chain is a human light chain. Preferably the light chain constant region is a human constant region. In the human population, multiple light chain constant region alleles exist. The nucleotide and amino acid sequences of these allelic variants are accessible on publicly available databases such as IMGT, ENSEMBL, Swiss-Prot and Uniprot. In one embodiment, the antibodies or antibody fragments disclosed herein comprise a protein encoded by a human κ constant region allele, which includes, but is not limited to, IGKC*01 (see, eg, the sequence table herein), IGKC*02 (see, eg, the sequence table herein), IGKC*03 (see, eg, the sequence table herein), IGKC*04 (see, eg, the sequence table herein) and IGKC*05 (see, eg, the sequence table herein). In one embodiment, the antibodies or antibody fragments disclosed herein comprise a protein encoded by a human A constant region allele, which includes but is not limited to IGLC1*01 (see, eg, the sequence table herein), IGLC1*02 (see, eg, the sequence table herein), IGLC2*01 (see, eg, the sequence table herein), IGLC2*02 (see, eg, the sequence table herein), IGLC2*03 (see, eg, the sequence table herein), IGLC3*01 (see, eg, the sequence table herein), IGLC3*02 (see, eg, the sequence table herein), IGLC3*03 (see, eg, the sequence table herein), IGLC3*04 (see, eg, the sequence table herein), IGLC6*01 (see, eg, the sequence table herein), IGLC7*01 (see, eg, the sequence table herein), IGLC7*02 (see, eg, the sequence table herein), IGLC7*03 (see, eg, the sequence table herein). In another embodiment, the antibodies and antibody fragments disclosed herein comprise a light chain constant region encoded by a mouse K constant region allele, which includes, but is not limited to, IGKC*01, IGKC*03 or IGKC*03. In another embodiment, the antibodies and antibody fragments disclosed herein comprise a light chain constant region encoded by a mouse A constant region allele, which includes, but is not limited to, IGLC1*01, IGLC2*01 or IGLC3*01.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEG ALIGN™ (DNASTAR) software. In one embodiment, the % homology is about 70%. In one embodiment, the % homology is about 75%. In one embodiment, the % homology is about 80%. In one embodiment, the % homology is about 85%. In one embodiment, the % homology is about 90%. In one embodiment, the % homology is about 92%. In one embodiment, the % homology is about 95%. In one embodiment, the % homology is about 97%. In one embodiment, the % homology is about 98%. In one embodiment, the % homology is about 99%. In one embodiment, the % homology is 100%.

The term “naturally occurring” or “native” when used in connection with biological materials such as nucleic acid molecules, polypeptides, host cells, and the like, refers to those which are found in nature and not manipulated by a human being.

As used herein, “packaging” refers to how the components are organized and/or restrained into a unit fit for distribution and/or use. Packaging can include, e.g. boxes, bags, syringes, ampoules, vials, tubes, clamshell packaging, barriers and/or containers to maintain sterility, labelling, etc.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

As used herein, the term “polynucleotide,” “nucleotide,” nucleic acid” “nucleic acid molecule” and other similar terms are used interchangeable and include DNA, RNA, mRNA and the like.

As used herein, the terms “prevent”, “preventing”, and “prevention” refer to the total or partial inhibition of the development, recurrence, onset or spread of a hCD7-mediated disease and/or symptom related thereto, resulting from the administration of a therapy or combination of therapies provided herein (e.g. a combination of prophylactic or therapeutic agents, such as an antibody of the invention).

The term “soluble” refers to a polypeptide, such as CD7 and variants or fragments thereof, that is lacking one or more transmembrane or cytoplasmic domains found in the native or membrane-associated form. In one embodiment, the “soluble” form of CD7 lacks both a transmembrane domain and cytoplasmic domain.

The term “subject” or “patient” refers to any animal, including, but not limited to, mammals. As used herein, the term “mammal” refers to any vertebrate animal that suckle their young and either give birth to living young (eutharian or placental mammals) or are egg-laying (metatharian or nonplacental mammals). Examples of mammalian species include, but are not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like.

As used herein “substantially all” refers to refers to at least about 60%, at least about 70%, at least about 75%, at least about 80%, at least about 85%, at least about 90%, at least about 95%, at least about 98%, at least about 99%, or about 100%.

As used herein, the term “therapeutic agent” refers to any agent that can be used in the treatment, management or amelioration of a CD7-mediated disease and/or a symptom related thereto. In certain embodiments, the term “therapeutic agent” refers to an antibody of the invention. In certain other embodiments, the term “therapeutic agent” refers to an agent other than an antibody of the invention. Preferably, a therapeutic agent is an agent which is known to be useful for, or has been or is currently being used for the treatment, management or amelioration of a CD7-mediated disease or one or more symptoms related thereto. In specific embodiments, the therapeutic agent is a fully human anti-CD7 antibody, such as a fully human anti-CD7 monoclonal antibody.

As used herein, the term “therapy” refers to any protocol, method and/or agent that can be used in the prevention, management, treatment and/or amelioration of a CD7-mediated disease (e.g. cancer). In certain embodiments, the terms “therapies” and “therapy” refer to a biological therapy, supportive therapy, and/or other therapies useful in the prevention, management, treatment and/or amelioration of a CD7-mediated disease known to one of skill in the art such as medical personnel. The terms “treat”, “treatment” and “treating” refer to the reduction or amelioration of the progression, severity, and/or duration of a hCD7-mediated disease (e.g. cancer) resulting from the administration of one or more therapies (including, but not limited to, the administration of one or more prophylactic or therapeutic agents, such as an antibody of the invention).

The term “variable region” or “variable domain” refers to a portion of the light and heavy chains, typically about the amino-terminal 120 to 130 amino acids in the heavy chain and about 100 to 110 amino acids in the light chain, which differ extensively in sequence among antibodies and are used in the binding and specificity of each particular antibody for its particular antigen. The variability in sequence is concentrated in those regions called complimentarily determining regions (CDRs) while the more highly conserved regions in the variable domain are called framework regions (FR). The CDRs of the CD7 and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5^{th} ed. (“Kabat et al.”). In preferred embodiments, the variable region is a human variable region.

Definitions of common terms in cell biology and molecular biology can be found in “The Merck Manual of Diagnosis and Therapy”, 19^{th} Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et al., ed., John Wiley and Sons, Inc.), Current Protocols in Cell Biology (CPCB) (Juan S. Bonifacino et al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties. Other terms are defined herein within the description of the various aspects of the invention.

### Target Rationale

CD7 is a 40 kDa transmembrane glycoprotein of the Ig superfamily (1). It is expressed on the surface of peripheral blood T-cells, NK cells, thymocytes, and bone marrow CD34⁺ CD38⁻ cells early during T-cell ontogeny (2). CD7 is expressed on most T cells except late memory T cells and effector CD8⁺ T cells. CD7 expression is reported during early T cell development. However, it is not expressed in the haemopoietic lineage stem cells (HSCs) (2,3), suggesting HSCs would not be affected by anti-CD7 antibody. While most peripheral T-cells are typically CD7 positive, the absence of CD7 from a small subset of circulating CD4⁺ memory cells (CD4⁺ CD45RA⁻ CD45R0) has been reported (4).

The natural ligand for CD7 has not yet been identified. CD7 has been demonstrated to act as a costimulatory molecule, and anti-CD7 monoclonal antibodies (mAbs) have been reported to be mitogenic, increase calcium flux and augment IL-2 production (5). CD7 binds to the phosphatidylinositol 3-kinase (PI 3-kinase) by means of a cytoplasmic tyrosine based YEDM motif and associates with a type II PI 4-kinase (6). However, the exact signalling mechanisms are still unknown. No major phenotypic abnormalities have been identified in CD7-deficient mice although some labs have reported relatively minor immune system defects such as reduced antigen-specific T-cell triggering, defective generation of antigen-specific cytotoxic T cells and protection from lipopolysaccharide-induced shock syndrome (7,8).

CD7 can be rapidly internalized following antibody binding as demonstrated on the human T-ALL cell line, CEM cells, where in excess of 50% of cell surface CD7 was internalized within 30 minutes (9), following ligation with an antibody. In a clinical study with the mouse-human chimeric antibody RFT2, the half-life of the antibody in humans was reported to be around 12 hours (10). Following internalization, the intracellular pathway of internalized CD7 is not well described as it can be recycled to the cell membrane or directly sent to lysosomes for degradation. This rapid internalization might affect the pharmacokinetic and pharmacodynamic profile of monoclonal antibodies in patients following treatment.

### CD7 in T-ALL and AML

CD7 is highly expressed on malignant immature T-cells and is generally absent on malignant mature T-cells, such as CD4⁺ Sezary leukaemia and HTLV-1⁺ adult T-cell leukaemia cells (11). In leukemic cells from diagnostic bone marrow samples obtained from patients with T-ALL, the median percentage of CD7 expression was >99% (12). High CD7 expression levels was also observed in samples collected from patients with relapsed T-ALL. CD7 expression level in leukemic cell at diagnosis or relapse consistently exceeded that measured in residual normal T cells in the same samples, and standard of care (SoC) chemotherapy does not affect CD7 expression in leukemic cells. In the bone marrow samples collected during chemotherapy that contained minimal residual disease (MRD), >99% of residual leukemic cells were CD7⁺. As CD7 expression levels remain high during therapy (12), CD7 is an excellent flow cytometry biomarker for diagnosis of T-ALL (Table 5).

In addition to T-ALL, CD7 is expressed on leukemic cells in 15% of acute myeloid leukaemia (AML) cases (13). CD7 expression in this subset of AML cases is correlated with loss of wild-type CCAAT/enhancer-binding protein alpha (CEBPA) gene due to mutations or silencing by epigenetic mechanisms (14). This mutation or epigenetic screening in AML has a potential clinical importance, allowing a subset of AML to be stratified for CD7-targeted therapy.

In summary, a cell-depletion strategy using anti-CD7 monoclonal antibody is promising to address an unmet medical need in adult T-ALL and CD7⁺ AML conditions, and also other CD7⁺ cancers.

Normal T-cell development is a strictly regulated process in which T progenitor cells migrate from the bone marrow to thymus and differentiate toward mature and functional T cells. During this differentiation process, dysregulation of oncogenes and tumour suppressor genes can drive immature thymocytes into uncontrolled clonal expansion and cause T-ALL (22). T-ALL accounts for about 20% of all cases of ALL and is more common in adults than children, although the incidence diminishes with increasing age. Patients typically present with a high white blood cell count and may also present with organmegaly, particularly mediastinal enlargement. Despite improved knowledge of T-ALL disease biology, standard of care (SoC) remains largely unchanged over the last decade, and still consists of intensive multi-agent chemotherapy potentially followed by autologous haematopoietic stem cell transplantation. With this SoC, 20% in children and 40% in adults receiving treatment relapse (23,24). Long term survival in the relapsed and refractory setting is very poor with fewer than 5% of patients subsequently achieving secondary responses. Many patients with refractory or relapsed B-cell lymphoblastic leukaemia have achieved complete remission with a prolonged survival rate after receiving the new generation of targeted therapies (25). However, there is little new drug developmental activity for the T-ALL treatment probably due to its relatively small patient population size.

T-ALL malignancies represent a group of hematologic cancers with high rates of relapse and mortality in patients for whom no effective targeted therapy exists. There is still a high unmet medical need to improve the clinical outcome of patients with relapsed and refractory T-ALL.

Thus, in one aspect the invention is useful for treating T-ALL, such as refractory T-ALL.

The invention, thus, provides various anti-CD7 antibodies and fragments (such as Fab or scFv fragments), uses, and methods. Examples are set out in the following numbered Clauses.
1. An antibody or fragment comprising a binding site which specifically binds to CD7 (Cluster of Differentiation 7), wherein the binding site comprises a VH domain that is encoded by a nucleotide sequence that is derived from the recombination of a human VH gene segment, DH gene segment and JH gene segment, wherein the VH gene segment is selected from IGHV3-15 and IGHV3-23.

In an example, the VH gene segment is IGHV3-15*01 or IGHV3-23*04. For example, the DH gene segment and JH gene segments are human gene segments.

In an example, specific binding is with a KD, K_{off} and/or Kₒₙ as described further below. In an example, specific binding is with a KD from 1 pM to 5 nM.

The skilled person is familiar with databases and other sources for human and other species of antibody gene segments. For example, the IMGT database is a suitable source, eg, the version as at 1 Sep. 2018.

Reference is made to the Examples, showing antibodies that are based on IGHV3-15 or IGHV3-23.

Surprisingly, these human VH gene segments produce anti-CD7 antibodies which have desirable anti-CD7 properties, such as those described in the Examples.
2. The antibody or fragment according to Clause 1, wherein the DH gene segment is a human gene segment selected from IGHD3-9, IGHD3-10 and IGH6-19.

In an example, the DH gene segment is a human gene segment selected from IGHD3-9*01, IGHD3-10*01 and IGH6-19*01.
3. The antibody or fragment according to Clause 1 or 2, wherein the JH gene segment is a human gene segment selected from IGHJ6, IGHJ4 and IGHJ5.

In an example, the JH gene segment is a human gene segment selected from IGHJ6*02, IGHJ4*02 and IGHJ5*02.
4. The antibody or fragment according to any preceding Clause, wherein the binding site comprises a CDRH3 sequence selected from SEQ ID NO: 3, 6, 23, 26, 43, 46, 63 and 66.
5. The antibody or fragment according to any preceding Clause, wherein the binding site comprises (i) a VH domain comprising SEQ ID NO: 7 paired with a VL domain comprising SEQ ID NO: 17; (ii) a VH domain comprising SEQ ID NO: 27 paired with a VL domain comprising SEQ ID NO: 37; (iii) a VH domain comprising SEQ ID NO: 47 paired with a VL domain comprising SEQ ID NO: 57; or (iv) a VH domain comprising SEQ ID NO: 67 paired with a VL domain comprising SEQ ID NO: 77.
6. The antibody or fragment according to any preceding Clause, wherein the binding site comprises a VH domain comprising SEQ ID NO: 7.
7. The antibody or fragment according to any preceding Clause, wherein the binding site comprises a VH domain comprising SEQ ID NO: 7 paired with a VL domain comprising SEQ ID NO: 17.
8. An antibody or fragment which specifically binds to CD7 and comprises the CDRH3 sequence of an anti-CD7 antibody according to any preceding Clause, or said CDRH3 sequence comprising 3, 2 or 1 amino acid substitution(s).
9. An antibody or fragment (optionally according to any preceding Clause) which specifically binds to CD7 and comprises a VH domain which comprises a CDRH3 sequence of an antibody selected from G09, F05, C02 and E04; or said sequence comprising 3, 2 or 1 amino acid substitution(s).
10. The antibody or fragment according to Clause 9, wherein the VH domain comprises (i) a CDRH3 sequence of an antibody selected from G09, F05, C02 and E04; or said CDRH3 sequence comprising 3, 2 or 1 amino acid substitution(s); and (ii) a CDRH1 sequence of said selected antibody; or said CDRH1 sequence comprising 3, 2 or 1 amino acid substitution(s).
11. The antibody or fragment according to Clause 9 or 10, wherein the VH domain comprises (iii) a CDRH3 sequence of an antibody selected from G09, F05, C02 and E04; or said CDRH3 sequence comprising 3, 2 or 1 amino acid substitution(s); and (iv) a CDRH2 sequence of said selected antibody; or said CDRH2 sequence comprising 3, 2 or 1 amino acid substitution(s).
12. An antibody or fragment (optionally according to any preceding Clause) comprising a binding site which specifically binds to CD7, wherein the binding site comprises a VH domain that comprises the amino acid sequence of a VH domain of an antibody selected from G09, F05, C02 and E04; or an amino acid that is at least 70% identical thereto.

For example, the identity is at least 85%. For example, the identity is at least 90%. For example, the identity is at least 95%.
13. The antibody or fragment according to Clause 9, 10, 11 or 12, wherein the selected antibody is G09.
14. The antibody or fragment according to any preceding Clause comprising first and second copies of said VH domain.

In an example, the antibody or fragment comprises a binding site comprising a VH domain of the invention paired with a VL domain of the invention, wherein the binding site is capable of specifically binding to CD7 (eg, mature CD7, eg human and/or cynomolgus monkey CD7). For example, the antibody or fragment comprise two of such binding sites.
15. An antibody or fragment (optionally according to any preceding Clause) comprising a binding site which specifically binds to CD7, wherein the binding site comprises a VL domain that is encoded by a nucleotide sequence that is derived from the recombination of a human VL gene segment and JL gene segment, wherein the VL gene segment is selected from IGKV1D-39, IGKV1-39, IGKV3-11, IGKV1-16 and IGKV1-5.

In an example, the VL gene segment is selected from IGKV1D-39*01, IGKV1-39*01, IGKV3-11*01, IGKV1-16*02 and IGKV1-5*03; or is selected from IGKV1D-39*01, IGKV3-11*01, IGKV1-16*02 and IGKV1-5*03.
16. The antibody or fragment according to Clause 15, wherein the VL is a VK and the JL gene segment is a human gene segment selected from IGKJ1 and IGKJ4.

In an example, the VL is a VK and the JL gene segment is a human gene segment selected from IGKJ1*01 and IGKJ4*01.
17. An antibody or fragment which specifically binds to CD7 and comprises the CDRL3 sequence of an anti-CD7 antibody according to any preceding Clause, or said CDRL3 sequence comprising 3, 2 or 1 amino acid substitution(s).
18. An antibody or fragment (optionally according to any preceding Clause) which specifically binds to CD7 and comprises a VL domain which comprises a CDRL3 sequence selected from SEQ ID NO: 13, 16, 33, 36, 53, 56, 73 and 76, or said selected CDRL3 sequence comprising 3, 2 or 1 amino acid substitution(s).
19. An antibody or fragment (optionally according to any preceding Clause) which specifically binds to Cluster of Differentiation 7 (CD7) and comprises a VL domain which comprises a CDRL3 (and optionally a CDRH3) sequence of an antibody selected from G09, F05, C02 and E04; or said sequence(s) each comprising 3, 2 or 1 amino acid substitution(s).
20. The antibody or fragment according to Clause 19, wherein the VL domain comprises (i) a CDRL3 sequence (and optionally a CDRH3) of an antibody selected from G09, F05, C02 and E04; or said CDR3 sequence(s) each comprising 3, 2 or 1 amino acid substitution(s); and (ii) a CDRL1 (and optionally a CDRH1) sequence of said selected antibody; or said CDR1 sequence(s) each comprising 3, 2 or 1 amino acid substitution(s).

Preferably, the selected antibody herein is G09 or comprises the variable domains of G09.
21. The antibody or fragment according to Clause 19 or 20, wherein the VL domain comprises (iii) a CDRL3 (and optionally a CDRH3) sequence of an antibody selected from G09, F05, C02 and E04; or said CDR3 sequence(s) each comprising 3, 2 or 1 amino acid substitution(s); and (iv) a CDRL2 (and optionally a CDRH2) sequence of said selected antibody; or said CDR2 sequence(s) each comprising 3, 2 or 1 amino acid substitution(s).
22. An antibody or fragment (optionally according to any preceding Clause) comprising a binding site which specifically binds to CD7, wherein the binding site comprises a VL domain that comprises the amino acid sequence of a VL domain of an antibody selected from G09, F05, C02 and E04; or an amino acid that is at least 70% identical thereto.

Optionally, there is provided an antibody or fragment (optionally according to any preceding Clause) comprising a binding site which specifically binds to Cluster of Differentiation 7 (CD7), wherein the binding site comprises a VL domain that comprises the amino acid sequence of a VL domain of an antibody selected from G09, F05, C02 and E04; or an amino acid that is at least 70, 80, 85, 90, 95, 96, 97, 98 or 99% identical thereto.

For example, the identity is at least 85%. For example, the identity is at least 90%. For example, the identity is at least 95%.

Optionally, the antibody or fragment comprises first and second copies of said VL domain.

In an example, the antibody or fragment comprises a binding site comprising a VL domain of the invention paired with a VH domain, wherein the binding site is capable of specifically binding to CD7 (eg, mature CD7, eg human and/or cynomolgus monkey CD7). For example, the antibody or fragment comprise two of such binding sites.
23. An antibody or fragment (optionally according to any preceding Clause) which specifically binds to Cluster of Differentiation 7 (CD7) and comprises the heavy chain amino acid sequence of an antibody selected from G09, F05, C02 and E04; or an amino acid that is at least 70, 80, 85, 90, 95, 96, 97, 98 or 99% identical thereto.

For example, the identity is at least 85%. For example, the identity is at least 90%. For example, the identity is at least 95%.
24. An antibody or fragment (optionally according to any preceding Clause) which specifically binds to Cluster of Differentiation 7 (CD7) and comprises the light chain amino acid sequence of an antibody selected from G09, F05, C02 and E04; or an amino acid that is at least 70, 80, 85, 90, 95, 96, 97, 98 or 99% identical thereto.

For example, the identity is at least 85%. For example, the identity is at least 90%. For example, the identity is at least 95%.
25. The antibody or fragment of Clause 23, comprising the light chain amino acid sequence of said selected antibody; or an amino acid that is at least 70, 80, 85, 90, 95, 96, 97, 98 or 99% identical thereto.

For example, the identity is at least 85%. For example, the identity is at least 90%. For example, the identity is at least 95%, 26. An antibody or fragment (optionally according to any preceding Clause) which specifically binds to a human CD7 epitope that is identical to an epitope to which the antibody of any preceding Clause binds.
27. The antibody or fragment according to Clause 26, wherein the epitope is identified by unrelated amino acid scanning, or by X-ray crystallography.

Contact amino acid residues involved in the interaction of antibody and antigen may be determined by various known methods to those skilled in the art.

In one embodiment, sequential replacement of the amino acids of the antigen sequence (using standard molecular biology techniques to mutate the DNA of the coding sequence of the antigen), in this case CD7 with Alanine (a.k.a Alanine scan), or another unrelated amino acid, may provide residues whose mutation would reduce or ablate the ability of the antibody to recognise the antigen in question. Binding may be assessed using standard techniques, such as, but not limited to, SPR, HTRF, ELISA (which are described elsewhere herein). Other substitutions could be made to enhance the disruption of binding such as changing the charge on the side chain of antigen sequence amino acids (e.g. Lysine change to glutamic acid), switching polar and non-polar residues (e.g. Serine change to leucine). The alanine scan or other amino substitution method may be carried out either with recombinant soluble antigen, or where the target is a cell membrane target, directly on cells using transient or stable expression of the mutated versions.

In one embodiment, protein crystallography may be used to determine contact residues between antibody and antigen (i.e. to determine the epitope to which the antibody binds), crystallography allows the direct visualisation of contact residues involved in the antibody-antigen interaction. As well as standard X-ray crystallography, cryo-electro microscopy has been used to determine contact residues between antibodies and HIV capsid protein (see Lee, Jeong Hyun, et al. “Antibodies to a conformational epitope on gp41 neutralize HIV-1 by destabilizing the Env spike.”, Nature communications, 6, (2015)).

In one embodiment, if the antibody recognises a linear epitope, short peptides based on the antigen sequence can be produced and binding of the antibody to these peptides can be assessed using standard techniques, such as, but not limited to, SPR, HTRF, ELISA (which are described elsewhere herein). Further investigation of the epitope could be provided by performing an Alanine scan on any peptides that show binding. Alternative to linear peptides, conformational scans could be carried out using Pepscan technology using their chemical linkage of peptides onto scaffolds, which has been used to determine discontinuous epitopes on CD20 targeting antibodies (Niederfellner, Gerhard, et al. “Epitope characterization and crystal structure of GA101 provide insights into the molecular basis for type I/II distinction of CD20 antibodies.”, Blood, 118.2, (2011), 358-367).

In one embodiment, limited proteolytic digestion and mass spectrophotometry can be used to identify binding epitopes. The antibody-antigen complex is digested by a protease, such as, but not limited to, trypsin. The digested complex peptides are compared to antibody-alone and antigen-alone digestion mass spectrophotometry to determine if a particular epitope is protected by the complexation. Further work involving amino acid substitution, competition binding, may then be employed to narrow down to individual amino acid residues involved in the interaction (see, for example, Suckau, Detlev, et al. “Molecular epitope identification by limited proteolysis of an immobilized antigen-antibody complex and mass spectrometric peptide mapping.”, Proceedings of the National Academy of Sciences, 87.24, (1990), 9848-9852).

Thus, in one embodiment, the contact residues of the epitope are identified with an unrelated amino acid scan (e.g. alanine scan). In another embodiment, an unrelated amino acid scan (e.g. alanine scan) is carried out using a technique selected from SPR, HTRF, ELISA, X-ray crystallography, cryo-electro microscopy and a combination of limited proteolytic digestion and mass spectrometry. In one embodiment, the unrelated amino acid scan (e.g. alanine scan) is carried out using HTRF. In one embodiment, the unrelated amino acid scan (e.g. alanine scan) is carried out using ELISA. When the alanine scan is carried out with either ELISA or HTRF, an amino acid residue is identified as contributing to the epitope if the reduction in signal is at least 25%. In one embodiment, the reduction in signal is at least 30%. In one embodiment, the reduction in signal is at least 35%. In one embodiment, the reduction in signal is at least 40%. In one embodiment, the reduction in signal is at least 45%. In one embodiment, the reduction in signal is at least 50%. In one embodiment, the reduction in signal is at least 55%. In one embodiment, the reduction in signal is at least 60%. In one embodiment, the reduction in signal is at least 70%. In one embodiment, the reduction in signal is at least 75%. In one embodiment, the reduction in signal is at least 80%. In one embodiment, the reduction in signal is at least 85%. In one embodiment, the reduction in signal is at least 90%. When the alanine scan is carried out with SPR, an amino acid residue is identified as contributing to the epitope if there is at least a 10-fold reduction in affinity. In one embodiment, the reduction in affinity is at least 15-fold. In one embodiment, the reduction in affinity is at least 20-fold. In one embodiment, the reduction in affinity is at least 30-fold. In one embodiment, the reduction in affinity is at least 40-fold. In one embodiment, the reduction in affinity is at least 50-fold. In one embodiment, the reduction in affinity is at least 100-fold.

In one embodiment, the contact residues of the epitope are identified by X-ray crystallography. In one embodiment, the contact residues of the epitope are identified by cryo-electro microscopy. In one embodiment, the contact residues of the epitope are identified by a combination of limited proteolytic digestion and mass spectrometry.
28. The antibody or fragment according to Clause 27, wherein the contact residues of the epitope are defined by a reduction in affinity of at least 10-fold in an unrelated amino acid scan, e.g. an alanine scan as determined by SPR.

In one embodiment, the reduction in affinity is at least 15-fold. In one embodiment, the reduction in affinity is at least 20-fold. In one embodiment, the reduction in affinity is at least 30-fold. In one embodiment, the reduction in affinity is at least 40-fold. In one embodiment, the reduction in affinity is at least 50-fold. In one embodiment, the reduction in affinity is at least 100-fold. SPR may be carried out as described herein.
29. An antibody or fragment (optionally according to any preceding Clause) which competes for binding to human CD7 with the antibody of any preceding Clause.

Optionally, competition is determined by surface plasmon resonance (SPR) or ELISA. The skilled person will be familiar with these techniques and standard conditions, for example.

In one embodiment, the antibody or fragment competes (e.g. in a dose-dependent manner) with hCD7 (or a fusion protein thereof) for binding to cell surface-expressed hCD7. In one embodiment, the antibody or fragment competes (e.g. in a dose-dependent manner) with hCD7 (or a fusion protein thereof) for binding to soluble hCD7.

Optionally, the competition for binding to hCD7 is conducted using SPR. SPR may be carried out as described herein.
30. The antibody or fragment according to any preceding Clause which specifically binds to human CD7 comprising SEQ ID NO: 82; and/or a cynomolgus CD7 comprising SEQ ID NO: 85; and/or a rat CD7 comprising SEQ ID NO: 86.

In an example, CD7 herein is a human, mouse or cynomolgus monkey CD7.

In one embodiment, the antibody or fragment binds to cynomolgus CD7 with an affinity of less than 1 nM (e.g. from 1 nM to 0.01 pM or from 1 nM to 0.1 pM, or from 1 nM to 1 pM). In one embodiment, the antibody or fragment binds to cynomolgus CD7 with an affinity of less than 10 nM (e.g. from 10 nM to 0.01 pM or from 10 nM to 0.1 pM, or from 10 nM to 1 pM). In one embodiment, the antibody or fragment binds to cynomolgus CD7 with an affinity of less than 0.1 nM (e.g. from 0.1 nM to 0.01 pM or from 0.1 nM to 0.1 pM, or from 0.1 nM to 1 pM). In one embodiment, the antibody or fragment binds to cynomolgus CD7 with an affinity of less than 0.01 nM (e.g. from 0.011 nM to 0.01 pM or from 0.01 nM to 0.1 pM).

In one embodiment, the antibody or fragment binds to cynomolgus CD7 with an affinity of within 2-fold of the affinity to hCD7. In one embodiment, the antibody or fragment binds to cynomolgus CD7 with an affinity of within 4-fold of the affinity to hCD7. In one embodiment, the antibody or fragment binds to cynomolgus CD7 with an affinity of within 5-fold of the affinity to hCD7. In one embodiment, the antibody or fragment binds to cynomolgus CD7 with an affinity of within 6-fold of the affinity to hCD7. In one embodiment, the antibody or fragment binds to cynomolgus CD7 with an affinity of within 8-fold of the affinity to hCD7. In one embodiment, the antibody or fragment binds to cynomolgus CD7 with an affinity of within 10-fold of the affinity to hCD7.

“hCD7” herein is a human CD7, eg, a human CD7 disclosed herein.

In one embodiment, the antibody or fragment does not detectably bind to cynomolgus CD7. In one embodiment, the antibody or fragment does not detectably bind to murine (eg, mouse and/or rat) CD7.

In one embodiment, the antibody or fragment binds to murine (eg, mouse and/or rat) CD7 with an affinity of less than 1 nM (e.g. from 1 nM to 0.01 pM or from 1 nM to 0.1 pM, or from 1 nM to 1 pM).

In one embodiment, the antibody or fragment binds to murine CD7 with an affinity of less than 10 nM (e.g. from 10 nM to 0.01 pM or from 10 nM to 0.1 pM, or from 10 nM to 1 pM). In one embodiment, the antibody or fragment binds to murine CD7 with an affinity of less than 0.1 nM (e.g. from 0.1 nM to 0.01 pM or from 0.1 nM to 0.1 pM, or from 0.1 nM to 1 pM). In one embodiment, the antibody or fragment binds to murine CD7 with an affinity of less than 0.01 nM (e.g. from 0.011 nM to 0.01 pM or from 0.01 nM to 0.1 pM).

Optionally, the antibody or fragment comprises an effector-enabled constant region, such as a human constant region, for example an IgG1 constant region. Optionally, the antibody or fragment comprises a murine (eg, mouse and/or rat) constant region. Optionally, the antibody or fragment comprises any of the heavy chain constant region sequences described herein.
31. The antibody or fragment according to any preceding Clause, wherein the antibody or fragment comprises a human constant region, e.g. an IgG1 constant region.

Optionally, the constant region is an IgG1 constant region, optionally the constant region comprises any IgG1 constant region amino acid sequence disclosed herein. Optionally, the constant region is an IgG2 constant region, optionally the constant region comprises any IgG1 constant region amino acid sequence disclosed herein. Optionally, the constant region is an IgG1 constant region, optionally the constant region comprises any IgG3 constant region amino acid sequence disclosed herein. Optionally, the constant region is an IgG1 constant region, optionally the constant region comprises any IgG4 constant region amino acid sequence disclosed herein.

In an example (optionally in addition to the heavy chain region as per the paragraph immediately above), the constant region comprises a light chain constant region, the light chain constant region comprising any light chain constant region amino acid sequence disclosed herein.
32. The antibody or fragment according to Clause 31, wherein the constant region is an IgG1 constant region, optionally the constant region comprises the amino acid sequence of SEQ ID NO: 88, 90, 92, 94 or 96 (eg, SEQ ID NO: 88).

In other embodiments, the antibody or fragment is any of the isotypes or constant regions as defined herein. In one embodiment, the constant region is wild-type human IgG1. For example, the constant region is an effector-enabled IgG1 constant region, optionally having ADCC and/or CDC activity. In one embodiment, the constant region is engineered for enhanced ADCC and/or CDC and/or ADCP. In another embodiment, the constant region is engineered for enhanced effector function.

The potency of Fc-mediated effects may be enhanced by engineering the Fc domain by any of the techniques as will be apparent to the skilled person. In another embodiment, the antibodies and fragments disclosed herein may comprise a triple mutation (M252Y/S254T/T256E) which enhances binding to FcRn.
33. The antibody or fragment according to any preceding Clause (eg, a bispecific antibody), further comprising an antigen-binding site that specifically binds another target antigen (optionally human CD5, CD14 or CD19, eg, for treating leukaemia, such as AML).

For example, the other target antigen is human CD5.

In an example, the further binding site is an agonist binding site for said another antigen. In an example, the further binding site is an antagonist binding site for said another antigen.

In an example, the further binding site is an antibody binding site comprising a VH and a VL; a binding site comprised by a constant domain of the antibody (eg, an Fcab binding site) or a non-immunoglobulin binding site (eg, a fibronectin domain). Optionally, the antigen-binding site is any antigen-binding site disclosed herein.

For example, the antibody or fragment is a bispecific antibody or fragment. For example, the antibody or fragment is a dual binding antibody or fragment, or a fusion protein comprising an antibody or fragment thereof as defined in any preceding Clause. A dual binding antibody has the meaning as set out above.

In an example, the antibody, fragment or fusion protein comprises a bispecific format selected from DVD-Ig™, mAb², FIT-Ig®, mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb®, scDiabody, scDiabody-CH₃, Diabody-CH₃, minibody, knobs-in-holes, knobs-in-holes with common light chain, knobs-in-holes with common light chain and charge pairs, charge pairs, charge pairs with common light chain, in particular mAb², knob-in-holes, knob-in-holes with common light chain, knobs-in-holes with common light chain and charge pairs and FIT-Ig®, e.g. mAb² and FIT-Ig®.

In one embodiment, the bispecific format is selected from DVD-Ig™, mAb², FIT-Ig®, mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb®, scDiabody, scDiabody-CH₃, Diabody-CH₃, Triple body, Miniantibody, minibody, TriBi minibody, scFv-CH₃ KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab™, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and Zybody™.

In one embodiment, the bispecific format is selected from DVD-Ig™, FIT-Ig®, mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb®, scDiabody, scDiabody-CH₃, Diabody-CH₃, Triple body, Miniantibody, minibody, TriBi minibody, scFv-CH₃ KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab™, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and Zybody™, for example DVD-Ig™, FIT-Ig®, mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb®, scDiabody, scDiabody-CH₃, Diabody-CH₃, minibody, knobs-in-holes, knobs-in-holes with common light chain, knobs-in-holes with common light chain and charge pairs, charge pairs, charge pairs with common light chain, in particular knob-in-holes, knob-in-holes with common light chain, knobs-in-holes with common light chain and charge pairs and FIT-Ig®, e.g. FIT-Ig®.

In one embodiment, the bispecific format is selected from DVD-Ig™, mAb², mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb®, scDiabody, scDiabody-CH₃, Diabody-CH₃, Triple body, Miniantibody, minibody, TriBi minibody, scFv-CH₃ KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab™, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and Zybody™, for example DVD-Ig™, mAb², mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb®, scDiabody, scDiabody-CH₃, Diabody-CH₃, minibody, knobs-in-holes, knobs-in-holes with common light chain, knobs-in-holes with common light chain and charge pairs, charge pairs, charge pairs with common light chain, in particular mAb², knob-in-holes, knobs-in-holes with common light chain and charge pairs, and knob-in-holes with common light chain, e.g. mAb².

In one embodiment, the bispecific format is selected from DVD-Ig™, mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb®, scDiabody, scDiabody-CH₃, Diabody-CH₃, Triple body, Miniantibody, minibody, TriBi minibody, scFv-CH₃ KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab™, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and Zybody™, for example DVD-Ig™, mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb®, scDiabody, scDiabody-CH₃, Diabody-CH₃, minibody, knobs-in-holes, knobs-in-holes with common light chain, knobs-in-holes with common light chain and charge pairs, charge pairs, charge pairs with common light chain, in particular knob-in-holes, knobs-in-holes with common light chain and charge pairs, and knob-in-holes with common light chain.
34. An anti-CD7 antibody or fragment as defined in any preceding Clause for treating or preventing a CD7-mediated disease or condition (optionally a cancer, such as a leukaemia or a lymphoma) in a subject.
35. The anti-CD7 antibody or fragment of Clause 34, wherein the disease or condition is selected from a leukaemia, a lymphoma, a blood cancer and a myelodysplastic syndrome (MDS).

In an example, the subject is a human. In an alternative, the subject is a non-human animal. In an example, the subject is an adult human. In an example, the subject is a paediatric human.

In an example, the antibody or fragment herein is for treating or preventing a disease or condition in a subject (eg, a human) selected from
T-cell acute lymphoblastic leukaemia;
Acute Myeloid Leukemia with CD7 expression;
Precursor T-Cell Lymphoblastic Lymphoma;
T-cell Prolymphocytic Leukemia;
T-cell Large Granular Lymphocytic Leukemia;
Peripheral T-cell Lymphoma;
Angioimmunoblastic T-cell Lymphoma;
Extranodal NK/T-cell Lymphoma, Nasal Type;
Enteropathy-type Intestinal T-cell Lymphoma; and
Hepatosplenic T-cell Lymphoma.

In an example, the disease or condition is in a human. In an example, the disease or condition is in an animal.

In an example, the antibody or fragment of the invention is for treating or preventing a CD7-mediated disease or condition in a human, e.g. selected from neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma.

In an example, the CD7-mediated disease or condition is a neurodegenerative disease, disorder or condition, e.g. selected from Alzheimer's disease, amyotrophic lateral sclerosis, Parkinson's disease, Huntington's disease, primary progressive multiple sclerosis, secondary progressive multiple sclerosis, corticobasal degeneration, Rett syndrome, a retinal degeneration disorder selected from age-related macular degeneration and retinitis pigmentosa; anterior ischemic optic neuropathy, glaucoma, uveitis, depression, trauma-associated stress or post-traumatic stress disorder, frontotemporal dementia, Lewy body dementias, mild cognitive impairments, posterior cortical atrophy, primary progressive aphasia and progressive supranuclear palsy or aged-related dementia, in particular, the neurodegenerative disease, disorder or condition is selected from Alzheimer's disease, amyotrophic lateral sclerosis, Parkinson's disease and Huntington's disease, for example, Alzheimer's disease.

In an example, the antibody, fragment, combination of the invention is administered intravenously to the subject; or is for administration intravenously to the subject. In an example, the antibody, fragment, combination of the invention is administered subcutaneously to the subject; or is for administration subcutaneously to the subject.
36. The antibody or fragment of Clause 33, wherein the antibody or fragment is administered to the subject simultaneously or sequentially with chemotherapy, radiotherapy or an immune checkpoint inhibitor.

Optionally, the chemotherapy is selected from nelarabine; cyclophosphamide; vincristine; adriamycin; and dexamethasone alternating with methotrexate and cytarabine.
37. A combination of an amount of an anti-CD7 antibody or fragment and an amount of a chemotherapeutic agent (optionally comprising multiple doses of said antibody and/or agent), wherein the antibody or fragment is according to any one of Clauses 1 to 36.

There is also provided: A medical kit comprising the combination, a first sterile container comprising said amount of antibody or fragment, and a second sterile container comprising said amount of a chemotherapeutic agent, and optionally instructions for using the combination to treat cancer in a subject.
38. The antibody, fragment or combination according to any one of Clauses 1 to 37 for use in a method of treating leukaemia in a human, wherein the antibody, fragment or combination is administered to the human optionally together with an antagonist of human CD5, CD14 or CD19.
39. The antibody, fragment or combination of Clause 38, wherein the leukaemia is acute myeloid leukaemia or T-ALL, or is a relapsed leukaemia (eg, relapsed AML or T-ALL).
40. The antibody, fragment or combination according to any one of Clauses 1 to 37 for use in a method of treating a disease or condition in a human or animal subject mediated by CD7⁺ cells in the subject, wherein the method comprises administering the antibody, fragment of combination to the subject wherein CD7 cells are targeted and killed, optionally by ADCP and/or CDC.
41. Use of the antibody, fragment or combination as defined in any preceding Clause in the manufacture of a medicament for administration to a subject for treating or preventing a CD7-mediated disease or condition, optionally a cancer.
42. A method of treating or preventing a CD7-mediated disease or condition in a subject (optionally a cancer), the method comprising administering to said subject a therapeutically effective amount of an antibody, fragment or combination as defined in any one of Clauses 1 to 40, wherein the CD7-mediated disease or condition is thereby treated or prevented.
43. The use according to Clause 41 or the method according to Clause 42, wherein the CD7-mediated disease or condition is a leukaemia (optionally T-ALL).
44. The antibody, fragment, combination, use or the method according to any one of Clauses 34 to 43, further comprising administering to the subject a further therapy, for example a further therapeutic agent, optionally wherein the further therapeutic agent is selected from the group consisting of:
   a. Cyclophosphamide;
   b. Vincristine;
   c. Adriamycin;
   d. Dexamethasone;
   e. Methotrexate;
   f. Cytarabine; and
   g. Nelarabine.
45. A pharmaceutical composition comprising an antibody, fragment or combination as defined in any one of Clauses 1 to 40 and 44 and a pharmaceutically acceptable excipient, diluent or carrier and optionally in combination with a further therapeutic agent selected from an agent recited in Clause 44.
46. The pharmaceutical composition according to Clause 45 for treating and/or preventing a CD7-mediated condition or disease, optionally a cancer.
47. The pharmaceutical composition according to Clause 45 or 46 in combination with a label or instructions for use to treat and/or prevent said disease or condition in a human; optionally wherein the label or instructions comprise a marketing authorisation number (optionally an FDA or EMA authorisation number); optionally wherein the kit comprises an IV or injection device that comprises the antibody or fragment.
48. A nucleic acid that encodes a VH domain and/or a VL domain of an antibody or fragment as defined in any one of Clauses 1 to 33.
49. A nucleic acid that encodes a VH domain comprising the amino acid sequence of a VH domain of an antibody selected from G09, F05, C02 and E04; or an amino acid that is at least 70, 80, 85, 90, 95, 96, 97, 98 or 99% identical thereto.

For example, the identity is at least 85%. For example, the identity is at least 90%. For example, the identity is at least 95%,
50. A nucleic acid that encodes a VL domain comprising the amino acid sequence of a VL domain of an antibody selected from G09, F05, C02 and E04; or an amino acid that is at least 70, 80, 85, 90, 95, 96, 97, 98 or 99% identical thereto.

Optionally, the nucleic acid also encodes a VH domain comprising the amino acid sequence of a VH domain of the selected antibody; or an amino acid that is at least 70, 80, 85, 90, 95, 96, 97, 98 or 99% identical thereto. For example, the identity is at least 85%. For example, the identity is at least 90%. For example, the identity is at least 95%.
51. A nucleic acid comprising
   (a) a nucleotide sequence that is at least 70% identical to the sequence of SEQ ID NO: 10; and/or
   (b) a nucleotide sequence that is at least 70% identical to the sequence of SEQ ID NO: 20.
52. A nucleic acid that encodes a heavy chain and/or a light chain of an antibody or fragment as defined in any one of Clauses 1 to 33.
53. A nucleic acid that encodes a heavy chain comprising an amino acid sequence that is at least 70% identical to SEQ ID NO: 8.
54. A nucleic acid that encodes a light chain comprising an amino acid sequence that is at least 70% identical to SEQ ID NO: 18.
55. A nucleic acid (eg, in a host cell, eg, a CHO or HEK293 or Cos cell) comprising
   (a) a nucleotide sequence that is at least 70% identical to a heavy chain sequence selected of an antibody selected from G09, F05, C02 and E04; and/or
   (b) a nucleotide sequence that is at least 70% identical to a sequence selected of an antibody selected from G09, F05, C02 and E04.

Herein in any instance where % identity is mentioned, in an example there is 100% identity.
56. A nucleic acid that encodes a heavy chain and/or a light chain of an antibody or fragment as defined in any one of Clauses 1 to 32.

All of the nucleic acids of the invention herein are expressible in a host cell, eg, a CHO or HEK293 or Cos cell, such as for expressing a variable domain or chain of an antibody or fragment of the invention.
57. A vector comprising the nucleic acid(s) (eg, the nucleic acid(s) of any one of Clauses 48 to 55); optionally wherein the vector is a CHO or HEK293 vector.
58. A host cell comprising the nucleic acid(s) (eg, the nucleic acid(s) of any one of Clauses 48 to 55) or the vector of Clause 57.
59. An antibody, fragment, combination, vector, host cell, composition, use or method according to any preceding Clause, wherein the antibody or fragment comprise an IgG1 (eg, a human IgG1 or IgG1*01) constant region.
60. An antibody, fragment, combination, vector, host cell, composition, use or method according to Clause 59, wherein the constant region comprises a glycine at position 430, an arginine at position 356 and/or an arginine at position 357 (according to EU numbering) in the IgG1 CH3 region.
61. An antibody, fragment, combination, vector, host cell, composition, use or method according to Clause 59, wherein the constant region comprises a glycine at position 430.
62. An antibody, fragment, combination, vector, host cell, composition, use or method as herein described.

the invention provides:

A method of diagnosing a CD7-mediated disease or condition in a subject (optionally a cancer), the method comprising combining an antibody or fragment of the invention with an isolated cell sample (eg, a blood or serum sample) and determining that cells comprised by the sample are specifically bound by the antibody or fragment.

Also provided is: An in vitro assay for detecting CD7-positive cells in a sample, the assay comprising combining an antibody or fragment of the invention with an isolated cell sample (eg, a blood or serum sample) and determining that cells comprised by the sample are specifically bound by the antibody or fragment.

The disease or condition may be any disease or condition disclosed herein. Detection may be by any conventional means, eg, using a label such as a fluorescence label, ELISA or a RIA.

In an example, the antibody or fragment comprises a HCDR3 length of 9, 10, 11 or 12 residues, eg, 10, eg, 11. In an example, the antibody or fragment comprises a LCDR3 length of 7, 8 or 9 residues, eg, 8, eg, 9. In an example, each VH domain of the antibody or fragment comprises from 1-11 non-germline residues, eg, 1, 2, 3, 4, 5, 6, 7, 8, 9, 10 or 11 non-germline residues. In an example, each VL domain of the antibody or fragment comprises from 3-8 non-germline residues, eg, 3, 4, 5, 6, 7 or 8 non-germline residues.

In an embodiment, a CDR sequence herein is determined according to Kabat. In an alternative, the CDR sequence is determined according to IMGT.

In an example, the selected antibody is G09.

In an example, the selected antibody comprises the heavy chain of G09, F05, C02 or E04. In an example, the selected antibody comprises the heavy chain of G09.

In an example, the heavy chain of the antibody or fragment of the invention is a human gamma-1, gamma-2, gamma-3, gamma-4, mu, delta, epsilon or alpha isotype, preferably a gamma isotype (eg, an IgG4 isotype). In an example, the light chain of the antibody or fragment of the invention comprises a human kappa constant region. Alternatively, in an example, the light chain of the antibody or fragment of the invention comprises a human lambda constant region.

Optionally, the antibody is a 4-chain antibody comprising a dimer of a heavy chain associated with a dimer of a light chain. In an example, the heavy chain comprises one or heavy chain CDRs or a CDR combination as disclosed herein and/or the light chain comprises one or heavy chain CDRs or a CDR combinations as disclosed herein, such as from the same selected antibody. In an example, the heavy chain comprises a VH domain as disclosed herein and/or the light chain comprises a VL as disclosed herein, such as from the same selected antibody. In an example, the heavy chain and the light chain are from the same selected antibody, eg, any antibody disclosed in the sequence table herein or the tables in the Examples herein.

In an example, the selected antibody comprises the light chain of G09, F05, C02 or E04. In an example, the selected antibody comprises the light chain of G09.

In an example, the selected antibody comprises the variable domains of G09, F05, C02 or E04. In an example, the selected antibody comprises the variable domains of G09.

In an example, the selected antibody comprises the VH domains of G09, F05, C02 or E04. In an example, the selected antibody comprises the VH domains of G09.

In an example, the selected antibody comprises the VH and VL domains of G09, F05, C02 or E04. In an example, the selected antibody comprises the VH and VL domains of G09.

In an example, the binding site of the antibody or fragment comprises a VH/VL pair that specifically binds to human CD7.

Optionally, the antibody or fragment competes with G09 (eg, G09 in IgG format, eg, human IgG1) for binding to CD7 as determined by SPR.

Optionally, the amino acid substitutions are conservative amino acid substitutions, optionally wherein each conservative substitution is from group (1) to (6):
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Any SPR herein is, for example, surface plasmon resonance (SPR) at 37° C. and pH 7.6.

Optionally, any CD7 herein is (for example, in in vitro testing) human CD7, eg, comprising the amino acid sequence of human CD7 disclosed herein.

In an example, the antibody or fragment of the invention binds to human CD7 with a Ka of eg, 5×10⁶ M⁻¹×s⁻¹; or about 5×10⁶ M⁻¹×s⁻¹. In an example, the antibody or fragment of the invention binds to human CD7 with a Kd of eg, 4 or 5 s⁻¹; or about 4 or 5 s⁻¹. In an example, the antibody or fragment of the invention binds to human CD7 with a KD of eg, 0.07 or 0.14 nM; or about 0.07 or 0.14 nM. In an embodiment, the fragment is a Fab fragment. In an embodiment, the fragment is a scFv.

Optionally, the antibody of the invention has an affinity (KD) for binding CD7 of from 1 pM to 5 nM, optionally wherein binding is determined by SPR using a Fab of said antibody at 37° C. at pH 7.6.

Optionally, the antibody has off-rate (K_{off}) for binding CD7 of from 1×10⁻⁵ to 1×10⁻³ S⁻¹, optionally wherein binding is determined by SPR using a Fab of said antibody at 37° C. at pH 7.6.

Optionally, the antibody has on-rate (Kₒₙ) for binding CD7 of from 1×10⁵ to 1×10⁷ M⁻¹S⁻¹, optionally wherein binding is determined by SPR using a Fab of said antibody at 37° C. at pH 7.6.

In an example, the antibody (eg, as a Fab) or fragment has an affinity (KD) for binding CD7 (eg, human CD7) of
(a) from 2, 3, 4, 5 or 10 pM to 3, 4 or 5 nM;
(b) from 1-10 pM to 5 nM;
(c) from 10 pM to 3, 4 or 5 nM;
(d) from 50 or 80 pM to 200 nM;
(e) from 50 or 80 pM to 150 nM; or
(f) from 50 or 80 pM to 100 nM.

In an example, the KD is (or is about) 5-15 pM (eg, 10 pM). In an example, the KD is (or is about) 2-5 nM (eg, 3 nM). In an example, the KD is (or is about) 100-400 pM (eg, 140 or 390 pM).

In an example, the antibody (eg, as a Fab) or fragment has an off-rate (K_{off}) for binding CD7 (eg, human CD7) of

$$from 1 \times {10}^{- 5} to 5 \times {10}^{- 4} {S}^{- 1} ; \qquad ( a )$$

$$from 1 \times {10}^{- 5} to 6 \times {10}^{- 4} {S}^{- 1} ; \qquad ( b )$$

$$from 1 \times {10}^{- 5} to 7 \times {10}^{- 4} {S}^{- 1} ; \qquad ( c )$$

$$from 1 \times {10}^{- 5} to 8 \times {10}^{- 4} {S}^{- 1} ; \qquad ( d )$$

$$from 2 \times {10}^{- 5} to 1 \times {10}^{- 3} {S}^{- 1} ; \qquad ( e )$$

$$from 2 \times {10}^{- 5} to 5 \times {10}^{- 4} {S}^{- 1} ; \qquad ( f )$$

$$from 2 \times {10}^{- 5} to 6 \times {10}^{- 4} {S}^{- 1} ; \qquad ( g )$$

$$from 2 \times {10}^{- 5} to 7 \times {10}^{- 4} {S}^{- 1} ; or \qquad ( h )$$

$$from 2 \times {10}^{- 5} to 8 \times {10}^{- 4} {S}^{- 1} . \qquad ( i )$$

In an example, the K_{off} is (or is about) 5×10⁻⁴ S⁻¹ (eg, when the KD is (or is about) from 2 nM to 400 pM; when the KD is (or is about) 2-5 nM (eg, 3 nM); or when the KD is (or is about) 100-400 pM (eg, 140 or 390 pM)). In an example, the K_{off} is (or is about) 3×10⁻⁵ S⁻¹ (eg, when the KD is (or is about) from 5-15 pM (eg, 10 pM)).

In an example, the antibody (eg, as a Fab) or fragment has an on-rate (Kₒₙ) for binding CD7 (eg, human CD7) of

$$from 1 \times {10}^{5} to 1 \times {10}^{6} {M}^{- 1} {S}^{- 1} ; \qquad ( a )$$

$$from 1 \times {10}^{5} to 2 \times {10}^{6} {M}^{- 1} {S}^{- 1} ; \qquad ( b )$$

$$from 1 \times {10}^{5} to 3 \times {10}^{6} {M}^{- 1} {S}^{- 1} ; \qquad ( c )$$

$$from 1 \times {10}^{5} to 4 \times {10}^{6} {M}^{- 1} {S}^{- 1} ; \qquad ( d )$$

$$from 1 \times {10}^{5} to 5 \times {10}^{6} {M}^{- 1} {S}^{- 1} ; \qquad ( e )$$

$$from 2 \times {10}^{5} to 5 \times {10}^{6} {M}^{- 1} {S}^{- 1} ; \qquad ( f )$$

$$from 3 \times {10}^{5} to 5 \times {10}^{6} {M}^{- 1} {S}^{- 1} ; \qquad ( g )$$

$$from 4 \times {10}^{5} to 5 \times {10}^{6} {M}^{- 1} {S}^{- 1} ; \qquad ( h )$$

$$from 5 \times {10}^{5} to 5 \times {10}^{6} {M}^{- 1} {S}^{- 1} ; or \qquad ( i )$$

$$from 6 \times {10}^{5} to 5 \times {10}^{6} {M}^{- 1} {S}^{- 1} . \qquad ( j )$$

In an example, the Kₒₙ is (or is about) 1 or 2×10⁻⁵ M⁻¹S⁻¹ (eg, when the KD is 2-5 nM (eg, 3 nM)). In an example, the Kₒₙ is (or is about) 1-4, 1, 2, 3 or 4×10⁻⁶ M⁻¹S⁻¹ (eg, when the KD is (or is about) from 5-400 pM (eg, 140 or 390 pM) or 5-15 pM (eg, 10 pM)).

As provided in the Clauses or other aspects herein, an anti-CD7 antibody or fragment may bind to CD7, e.g. human CD7 with a K_{D} of less than 50 nM, less than 40 nM, less than 30 nM as determined by surface plasmon resonance. Another embodiment, anti-CD7 antibody or fragment may bind to CD7, e.g. human CD7 with a K_{D} of less than 20 nM, less than 15 nM, less than 10 nM as determined by surface plasmon resonance. The anti-CD7 antibody or fragment may bind to CD7, e.g. human CD7 with a K_{D} of less than 8 nM, less than 5 nM, less than 4 nM, less than 3 nM, less than 2 nM or less than 1 nM as determined by surface plasmon resonance. The K_{D} may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less.

In another embodiment, the K_{D} is within a range of 0.01 to 1 nM, or a range of 0.05 to 2 nM, or a range of 0.05 to 1 nM. The K_{D} may be with regard to hCD7, cynomolgus monkey (ie, “cyno”) CD7 and/or mouse CD7.

In another embodiment, the anti-CD7 antibodies described herein have a K_{ON} rate (e.g. as measured by SPR, e.g. at 25° C. or at 37° C.) of approximately 0.5 to 10 μM, for example approximately 1 to 8 μM or approximately 1 to 7 μM. In another embodiment, the K_{ON} rate is approximately 1 to 5 μM, e.g. approximately 1 μM, approximately 1.5 μM, approximately 2 μM, approximately 2.5 μM or approximately 3 μM. In another embodiment, the K_{ON} rate is approximately 3.5 μM, approximately 4 μM, approximately 4.5 μM, approximately 5 μM or approximately 5.5 μM.

In another embodiment, the anti-CD7 antibodies described herein have a K_{OFF} rate (e.g. as measured by SPR, e.g. at 25° C. or at 37° C.) of approximately 0.01 to 100 mM, for example approximately 0.1 to 50 mM or approximately 0.5 to 50 mM. In another embodiment, the K_{OFF} rate is approximately 0.5 to 10 mM, or approximately 0.5 to 10 mM, e.g. approximately 1 mM, approximately 2 mM, approximately 3 mM, approximately 4 mM or approximately 5 mM. In another embodiment, the K_{OFF} rate is approximately 0.6 mM, approximately 0.7 mM, approximately 0.8 mM or approximately 0.9 mM.

In an example, the invention antibody or fragment comprises the VH and VL domains of G09, F05, C02 and E04.

In an example, the invention antibody or fragment comprises the VH and VL domains of G09. In an example, the invention antibody or fragment comprises the VH and VL domains of F05. In an example, the invention antibody or fragment comprises the VH and VL domains of G09. In an example, the invention antibody or fragment comprises the VH and VL domains of C02. In an example, the invention antibody or fragment comprises the VH and VL domains of E04.

In an example, the selected antibody is G09.

In an example, the selected antibody comprises the variable domains of an antibody selected from G09, F05, C02 and E04.

In an example, the selected antibody comprises the VH domains of an antibody selected from G09, F05, C02 and E04. In an example, the selected antibody comprises the VH domains of G09.

In an example, the selected antibody comprises the VH and VL domains of an antibody selected from G09, F05, C02 and E04. In an example, the selected antibody comprises the VH and VL domains of G09.

Optionally, the antibody or fragment of the invention comprises the HCDR3 of an antibody selected from G09, F05, C02 and E04. Optionally, the antibody or fragment of the invention comprises the HCDR1 and/or HCDR2 of said selected antibody.

Optionally the antibody or fragment of the invention comprises the HCDR1 of an antibody selected from G09, F05, C02 and E04. Optionally, the antibody or fragment of the invention comprises the HCDR2 and/or HCDR3 of said selected antibody.

Optionally the antibody or fragment of the invention comprises the HCDR2 of an antibody selected from G09, F05, C02 and E04. Optionally, the antibody or fragment of the invention comprises the HCDR1 and/or HCDR3 of said selected antibody.

Optionally, the antibody or fragment of the invention comprises the VH of an antibody selected from G09, F05, C02 and E04. Optionally, the antibody or fragment of the invention comprises the VL of said selected antibody.

Optionally, the antibody or fragment of the invention comprises the VL of an antibody selected from G09, F05, C02 and E04. Optionally, the antibody or fragment of the invention comprises the VH of said selected antibody.

Optionally, the antibody or fragment of the invention comprises the heavy chain of an antibody selected from G09, F05, C02 and E04. Optionally, the antibody or fragment of the invention comprises the light chain of said selected antibody.

Optionally, the antibody or fragment of the invention comprises the light chain of an antibody selected from G09, F05, C02 and E04. Optionally, the antibody or fragment of the invention comprises the heavy chain of said selected antibody. In an example, the selected antibody is G09.

Optionally, the antibody of the invention comprises a human IgG1*01 constant region. Optionally, the antibody of the invention comprises a human IgG1 E430G constant region, eg, an IgG1*01 E430G constant region. Optionally, the antibody of the invention comprises a human IgG1 E345R constant region, eg, an IgG1*01 E345R constant region.

Preferably, an antibody or a fragment thereof that specifically binds to a hCD7 does not cross-react with other antigens (but may optionally cross-react with different CD7 species, e.g., rhesus, cynomolgus, or murine). An antibody or a fragment thereof that specifically binds to a CD7 antigen can be identified, for example, by immunoassays, BIAcore™, or other techniques known to those of skill in the art. An antibody or a fragment thereof binds specifically to a hCD7 antigen when it binds to a hCD7 antigen with higher affinity than to any cross-reactive antigen as determined using experimental techniques, such as radioimmunoassays (RIA) and enzyme-linked immunosorbent assays (ELISAs). Typically, a specific or selective reaction will be at least twice background signal or noise and more typically more than 10 times background. See, e.g. Paul, ed., 1989, Fundamental Immunology Second Edition, Raven Press, New York at pages 332-336 for a discussion regarding antibody specificity.

Contact amino acid residues involved in the interaction of antibody and antigen, such as CD7, may be determined by various known methods to those skilled in the art.

In one embodiment, if the antibody recognises a linear epitope, short peptides based on the antigen sequence can be produced and binding of the antibody to these peptides can be assessed using standard techniques.

In one embodiment, limited proteolytic digestion and mass spectrophotometry can be used to identify binding epitopes.

In one embodiment, the contact residues of the epitope are identified by X-ray crystallography. In one embodiment, the contact residues of the epitope are identified by cryo-electro microscopy. In one embodiment, the contact residues of the epitope are identified by a combination of limited proteolytic digestion and mass spectrometry.

In another embodiment, the anti-CD7 antibodies (and fragments) described in herein provide improved transient expression levels over other anti-CD7 antibodies and fragments. Thus, in one embodiment, the anti-CD7 antibody (or fragment) is expressed in a HEK293 cell, e.g. a HEK293T cell, at an expression level of approximately 100 μg/mL, or in a range of approximately 100 to 350 μg/mL.

In another embodiment, the expression level is above approximately 350 μg/mL.

In another embodiment, the anti-CD7 antibody (or fragment) is expressed in a CHO cell, e.g. an Expi-CHO cell, at an expression level of approximately 100 μg/mL, or in a range of approximately 100 to 350 μg/mL. In another embodiment, the expression level is above approximately 350 μg/mL.

In another embodiment, the anti-CD7 antibody (or fragment) is expressed in a CHO cell, e.g. an Expi-CHO cell or a CHO-E7 EBNA cell, at an expression level of approximately 100 μg/mL, or in a range of approximately 100 to 350 μg/mL. In another embodiment, the expression level is above approximately 350 μg/mL. The antibody for example, comprises the VH and VL domains of any one of G09, formatted as a human IgG1 or human IgG4 (eg, IgG4-PE).

In any of these expression systems, the expression is carried out of a scale of between approximately 0.5 mL and 3 mL, for example between approximately 0.5 mL and 2 mL. In any of these expression systems, the anti-CD7 antibody (or fragment) may be expressed from a pTT5 vector. In any of these expression systems, the anti-CD7 antibody (or fragment) may be expressed in conjunction with a lipid transfection reagent, and may optionally be expressed in a CHO cell, e.g. an Expi-CHO cell. In any of these expression systems, the anti-CD7 antibody (or fragment) may be expressed in conjunction with a PEI transfection reagent, and may optionally be expressed in a CHO cell, e.g. an CHO-E7 EBNA cell. In any of these expression systems, the anti-CD7 antibody (or fragment) may be expressed in conjunction with a helper plasmid (e.g. an AKT helper plasmid), and may optionally be expressed in a CHO cell, e.g. an CHO-E7 EBNA cell.

In any of these expression systems, the expression level is between approximately 100 μg/mL and approximately 1500 μg/mL, for example between approximately 100 μg/mL and approximately 1000 μg/mL, or between approximately 200 μg/mL and approximately 1000 μg/mL, or between approximately 350 μg/mL and approximately 1000 μg/mL. In any of these expression systems, the lower limit of expression may be approximately 100 μg/mL, approximately 200 μg/mL, approximately 300 μg/mL, or approximately 400 μg/mL. In another embodiment, the lower limit of expression may be approximately 500 μg/mL, approximately 600 μg/mL, approximately 700 μg/mL, or approximately 800 μg/mL. In any of these expression systems, the upper limit of expression may be approximately 2000 μg/mL, approximately 1800 μg/mL, approximately 1600 μg/mL, or approximately 1500 μg/mL. In another embodiment, the upper limit of expression may be approximately 1250 μg/mL, approximately 1000 μg/mL, approximately 900 μg/mL, or approximately 800 μg/mL.

In another embodiment, the expression system is a Lonza expression system, e.g. Lonza X-Ceed® system. In the Lonza expression system, the expression may be carried out at a scale of approximately 30 mL to 2 L, for example 50 mL to 1 L, or 1 L to 2 L. In the Lonza expression system, the anti-CD7 antibody (or fragment) may be expressed in conjunction with electroporation, and optionally without any helper plasmids. In the Lonza expression system, the anti-CD7 antibody (or fragment) may be expressed at a level of approximately 1 g/L, or approximately 900 mg/L, or approximately 800 mg/L, or approximately 700 mg/L. In another embodiment, In the Lonza expression system, the anti-CD7 antibody (or fragment) may be expressed at a level of approximately 600 mg/L or approximately 500 mg/L or approximately 400 mg/L. In the Lonza expression system, the anti-CD7 antibody (or fragment) may be expressed at a level of between approximately 400 mg/L and approximately 2 g/L, for example between approximately 500 mg/L and approximately 1.5 g/L, or between approximately 500 mg/L and approximately 1 g/L. In another embodiment, the expression level is above 1 g/L. In another embodiment, the anti-CD7 antibodies provide improved half-life over other anti-CD7 antibodies.

In one embodiment, the antibody or fragment is a human antibody or fragment. In one embodiment, the antibody or fragment is a fully human antibody or fragment. In one embodiment, the antibody or fragment is a fully human monoclonal antibody or fragment.

in one embodiment, the antibody or fragment is a humanised antibody or fragment. In one embodiment, the antibody or fragment is a humanised monoclonal antibody or fragment.

Contact amino acid residues involved in the interaction of antibody and antigen may be determined by various known methods to those skilled in the art, such as alanine scanning, protein crystallography, mass spectrophotometry or any other technique as will be apparent to the skilled addressee.

In one embodiment, the recited CDR comprises one amino acid substitution, which may be a conservative amino acid substitution. In one embodiment, the recited CDR comprises two amino acid substitutions, which may be conservative amino acid substitutions. In one embodiment, the recited CDR comprises three amino acid substitutions, which may be conservative amino acid substitutions. In one embodiment, the recited CDR comprises four amino acid substitutions, which may be conservative amino acid substitutions. In one embodiment, the recited CDR comprises five amino acid substitutions, which may be conservative amino acid substitutions. In one embodiment, the recited CDR comprises six amino acid substitutions, which may be conservative amino acid substitutions.

Amino acid substitutions include alterations in which an amino acid is replaced with a different naturally-occurring amino acid residue. Such substitutions may be classified as “conservative”, in which case an amino acid residue contained in a polypeptide is replaced with another naturally occurring amino acid of similar character either in relation to polarity, side chain functionality or size.

Such conservative substitutions are well known in the art. Substitutions encompassed by the present invention may also be “non-conservative”, in which an amino acid residue which is present in a peptide is substituted with an amino acid having different properties, such as naturally-occurring amino acid from a different group (e.g. substituting a charged or hydrophobic amino; acid with alanine), or alternatively, in which a naturally-occurring amino acid is substituted with a non-conventional amino acid.

In one embodiment, the conservative amino acid substitutions are as described herein. For example, the substitution may be of Y with F, T with S or K, P with A, E with D or Q, N with D or G, R with K, G with N or A, T with S or K, D with N or E, I with L or V, F with Y, S with T or A, R with K, G with N or A, K with R, A with S, K or P. In another embodiment, the conservative amino acid substitutions may be wherein Y is substituted with F, T with A or S, I with L or V, W with Y, M with L, N with D, G with A, T with A or S, D with N, I with L or V, F with Y or L, S with A or T and A with S, G, T or V.

### ASPECTS

Any of the following Aspects may be combined with any of the features disclosed herein (eg, with any of the claimed embodiments herein or any of the Clauses herein). For example, the ligand in any of these Aspects may be an antibody or fragment of the invention.
1. An anti-CD7 ligand for administration to a human patient for the treatment of a cancer by complement-dependent cytotoxicity (CDC) of CD7⁺ cells (eg, cancer cells) in the patient, the ligand comprising an antibody Fc region and a binding site for specifically binding to human CD7.

In an example, the CD7 is human CD7 and the patient is a human.

In an alternative in any Aspect, instead of cancer, the ligand herein is for treating a disease or condition mediated by CD7+ cells, eg, CD7+ T-cells or NK cells. For example, the disease or condition is an autoimmune disease or condition. For example, the disease is graft-versus-host disease (GvHD). For example, the disease or condition is an inflammatory disease or condition.

Optionally, in an alternative in any Aspect the ligand is administered prophylactically to the subject to reduce the risk of cancer or the disease or condition.

Optionally, in any Aspect the cells are cells of the patient's immune system. Optionally, in any Aspect the cells are T- and/or NK-cells. Optionally, in any Aspect the cells are cells of a tissue, cell or organ transplant comprised by the human.
2. An anti-CD7 ligand for administration to a human patient for the treatment of a cancer by ligand-dependent phagocytosis of CD7⁺ cells (eg, cancer cells) in the patient, the ligand comprising an antibody Fc region and a binding site for specifically binding to human CD7.
3. Wherein the phagocytosis is antibody-dependent cellular phagocytosis (ADCP), wherein the antibody is said ligand.

In vivo, ADCP can be mediated by monocytes, macrophages, neutrophils and dendritic cells via FcγRIIa, FcγRI and FcγRIIIa. While all three receptors can participate in ADCP, FcγRIIa is believed to be the predominant Fcγ receptor involved in this process. In an example, the ADCP comprises phagocytosis of CD7⁺ cancer cells by macrophages and/or monocytes comprised by the patient.
4. An anti-CD7 ligand for administration to a human patient for the treatment of a cancer by ligand-dependent cell-mediated cytotoxicity of CD7⁺ cancer cells in the patient, the ligand comprising an antibody Fc region and a binding site for specifically binding to human CD7.

In ADCC, cytotoxicity may be mediated by natural killer (NK) cells; but macrophages, neutrophils and eosinophils can also mediate it. In an embodiment of the invention, ADCC may comprise ADCC by CD16+ immune cells of the patient. In an embodiment of the invention, ADCC may comprise ADCC by cells selected from natural killer (NK) cells; but macrophages, neutrophils and eosinophils.
5. The ligand of any preceding Aspect, wherein the cytotoxicity is antibody dependent cell-mediated cytotoxicity (ADCC), wherein the antibody is said ligand.
6. The ligand of any preceding Aspect, wherein the ligand comprises an anti-CD7 antibody, antibody fragment or trap.

In an example, the ligand comprises a paired VH/VL anti-CD7 binding site wherein the VH and VL are human antibody variable domains. Additionally or alternatively, the antibody or fragment comprises a human Fc.

In an example, the ligand is a human ligand, eg, a human antibody or fragment.

In an example, the ligand is capable of being internalised by CD7+ cells. In an example, the ligand is capable of being internalised by the cancer cells. In an example, the ligand is capable of being internalised by CEM cells in vitro.
7. The ligand of any preceding Aspect, wherein the patient has previously received a cancer chemotherapy agent.

In an example, the patient has previously received an immune checkpoint inhibitor, eg, an antibody against an immune checkpoint inhibitor. In an example, the inhibitor is ipilimumab, nivolumab, pembrolizumab or tremelimumab.

In an example, the patient has previously received anti-cancer radiation treatment.
8. The ligand of any preceding Aspect, wherein the patient has previously received GCSF.

Chemotherapy can cause myelosuppression and unacceptably low levels of white blood cells (neutropenia), making patients susceptible to infections and sepsis. GCSF stimulates the production of granulocytes, a type of white blood cell. In oncology and hematology, a recombinant form of GCSF is used with certain cancer patients to accelerate recovery from neutropenia after chemotherapy, allowing higher-intensity treatment regimens. It may be administered to oncology patients via subcutaneous or intravenous routes. In the context of the present invention, administration of GCSF simultaneously or sequentially (eg, before) administering the anti-CD7 ligand to the patient may be beneficial for up-regulating cell types involved in the CDC, ADCC and ADCP-mediated CD7⁺ cell killing of the invention
9. The ligand of any preceding Aspect, wherein the patient has previously undergone chemotherapy and wherein immediately before administration of the ligand to the patient, the patient has received a treatment to enhance complement (eg, C1q) activity.
10. The ligand of any preceding Aspect, wherein the patient has received an administration of one or more complement components (eg, a composition comprising C1q).

For example, the components are comprised by blood or plasma which is administered to the patient.
11. Comprising determining the level or activity of complement (eg, C1q) in the patient before administration of said ligand

For example, C1q level is serum concentration in the patient in the range from 70 to 160 micrograms/ml, eg, as determined by quantitative ELISA, such as a sandwich ELISA. An example of a suitable technique for determination is set out in Biotechnol J. 2009 August; 4(8):1210-4. doi: 10.1002/biot.200800273, “Systemic lupus erythematosus and C1q: A quantitative ELISA for determining C1q levels in serum”, Dillon S P et al. In an embodiment, said range is 100 to 160 micrograms/ml.

The range of complement level for a normal healthy person:
1. The total complement levels: 41-90 haemolytic units
2. The C1 levels: 16-33 mg/dL
3. The C3 levels: 88-252 mg/dL for males; 88-206 mg/dL for female
4. The C4 levels: 12-72 mg/dL for males; 13-75 mg/dL for females

In an example, the invention comprises the administration of an anti-CD7 ligand with anti-CD46, anti-CD55 or anti-CD59 therapy in the patient to neutralize complement regulatory protein (CRP) function.
12. Comprising administering said ligand to the patient, whereby said CD7⁺ cancer cells are killed, and thereafter carrying out chemotherapy of the patient.
13. The ligand of any preceding Aspect, wherein the cancer cells are immune cells of the patient.
14. The ligand of any preceding Aspect, wherein the cancer cells are T-cells, NK cells, thymocytes or bone marrow CD34⁺ CD38⁻ cells.

The cancer cells are, eg, CD7⁺ CD34⁺CD2 T-cells. The cancer cells are, eg, CD34⁺CD38⁻ immune cells (eg, T-cells). The cancer cells are, eg, CD34⁺CD38⁺ immune cells (eg, T-cells).
15. The ligand of any preceding Aspect, wherein the T-cells are immature T-cells.

Optionally, the immature T-cells comprise one, two or more of the following types: DN1, DN2, DN3, DN4 and DP. DN1 cells are positive for the following markers: CD34, CD44, CD117, TdT, HLA-DR. DN2 cells are positive for the following markers: CD2, CD5, CD7, CD25, CD38, CD44, CD117, CD127, TdT, HLA-DR. DN3 cells are positive for the following markers: CD2, CD5, CD7, CD25, CD38, CD44, CD71, CD117, TdT. DN4 cells are positive for the following markers: CD1, CD2, CD5, CD7, CD38, TdT. DP cells are positive for the following markers: CD2, CD3, CD4 or CD8, CD7.

In an embodiment, the cancer cells comprise early thymic precursor (ETP) cells. The presence of such cells has been associated with high leukaemia risk and low or no response to Nelarabine. Thus, in an embodiment, the patient is Nelarabine refractory, eg, wherein the cancer cells comprise ETP cells.

In an example the cancer cells are CD52+.

Optionally, the immature T-cells are CD2⁺, CD5⁺, CD7⁺.
16. The ligand of any preceding Aspect, wherein the T-cells are CD7⁺ CD34⁺ CD38⁻ T-cells.

Optionally, the cells are, eg, CD7⁺ CD34⁺ CD38⁻ Lin⁻ T-cells, such as wherein the cancer is AML.

Optionally, the T-cells are CD8+ T-cells. Wherein the T-cells are CD4+ T-cells.

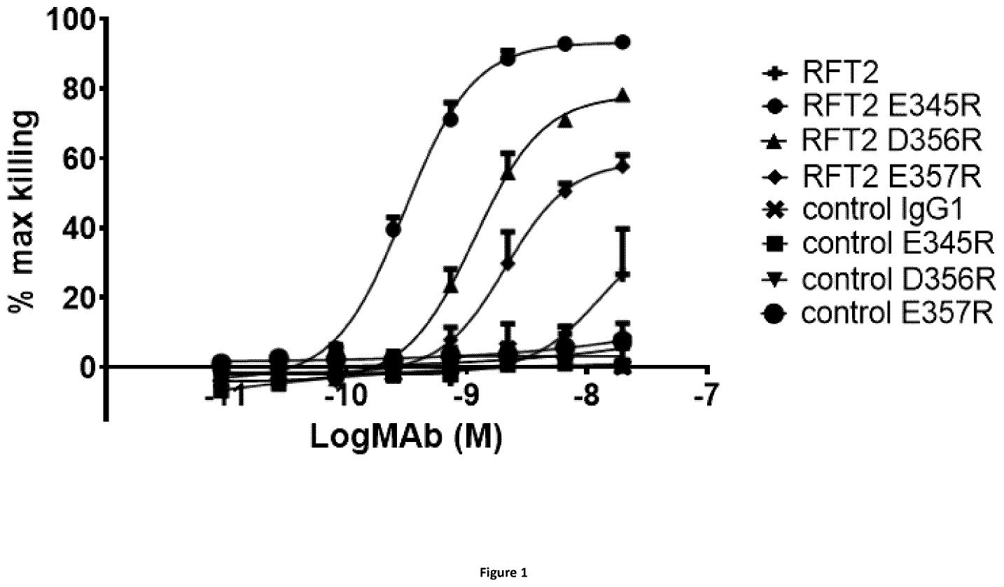

Optionally, the cells comprise a plurality of cells, each cell of said plurality comprising at least 100, 500 or 1000 copies of cell-surface CD7 (see, eg, FIG. 1a, Aandahl, E M et al. J Immunol. 2003. 170: 2349-2355 for guidance on such determination).
17. The ligand of any preceding Aspect, wherein the cells are leukaemia-initiating cells (LICs).
18. The ligand of any preceding Aspect, wherein the cells are leukaemia cells, eg, AML or T-ALL cells.
19. The ligand of any preceding Aspect, wherein the cancer cells are T-cells and NK cells are spared killing in the patient.
20. The ligand of any preceding Aspect, wherein the ligand kills no more than 70% (eg, no more than 80, 90 or 95%) of NK cells in an in a standard vitro cell killing test.
21. The ligand of any preceding Aspect, wherein the Fc region is a human Fc.
22. The ligand of any preceding Aspect, wherein the Fc region is a wild-type Fc.
23. The ligand of any preceding Aspect, wherein the Fc region is a human IGHG1*01 or IGHG1*02 nucleotide sequence.
24. The ligand of any preceding Aspect, wherein the cancer is acute myeloid leukaemia (AML), T-ALL (eg, patient-derived T-ALL), peripheral T cell lymphoma (PTCL) or T-cell prolymphocytic leukaemia (TPLL).

Optionally, the AML is M1/M2 AML. Optionally, the cancer is Mixed lineage leukaemia (MLL)-rearranged human acute lymphoblastic leukemia.

Optionally, the cancer is a cancer mediated by CD7⁺ immune cells (eg, T-cells).

Optionally, the cancer is lymphoblastic leukemia (LL) (eg, ALL or acute lymphocytic leukemia), Cutaneous T-cell lymphoma (CTCL) or melanoma. Optionally, the cancer is relapsed T-ALL or AML.

Optionally, the cancer is a liver cancer. Optionally, the cancer is selected from melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma.
25. The ligand of any preceding Aspect, wherein the AML is CCAAT/enhancer binding protein a (CEBPA) mutated AML.

Optionally, the patient is homozygous for CEBPA mutation.
26. The ligand of any preceding Aspect, wherein the patient has an immunophenotype of HLA-DR′ CD7⁺ CD13⁺ CD14⁻ CD15⁺ CD33⁺ CD34⁺, eg, wherein the cancer is AML, eg, CEBPA AML.
27. The ligand of any preceding Aspect, wherein the human is an adult.

For example, the adult is at least 18, 20, 30, 40, 50, 60, 70, 80 or 90 years' of age.
28. The ligand of any preceding Aspect, wherein the human is an infant.

For example, the human is a child, eg, a human under 18 years' of aged, eg, less than 17, 16, 15, 14, 13, 12, 11, 10, 9, 8, 7, 6, 5, 4, 3, 2 or 1 year of age.
29. The ligand of any preceding Aspect, wherein the patient is suffering from 1st or 2nd relapse of said cancer following previous treatment, eg, with a chemotherapy agent.

In an example, the agent comprises an immune checkpoint inhibitor, such as an inhibitor described herein.
30. The ligand of any preceding Aspect, wherein the patient has previously received Nelarabine.
31. The ligand of any preceding Aspect, wherein the ligand and Nelarabine are simultaneously or sequentially administered to the patient.

The invention may, for example, enable the administration of a lower dose than standard of care with Nelarabine.
32. The ligand of any preceding Aspect, wherein the ligand is administered to the patient simultaneously or sequentially with a haematopoietic stem cell transplant.

In an example, the transplant is an allogeneic transplant.

In an example, the ligand is administered no more often than once every 2 or 4 weeks. In an example, the ligand is administered fortnightly, monthly or weekly.
33. The ligand of any preceding Aspect, wherein the treatment reduces cancer progression in the patient.
34. The ligand of any preceding Aspect, wherein the treatment enhances cancer survival in human patients.
35. The ligand of any preceding Aspect, wherein the treatment causes cancer remission in the patient.
36. The ligand of any preceding Aspect, wherein said ligand mediates ADCP in a standard ADCP test.
37. The ligand of any preceding Aspect, wherein said ligand mediates CDC killing of CEM cells in an in vitro CEM cell killing assay with an EC₅₀ in the range from 10 to 500 pM (eg, from 10 to 500 or 100 pM).

CEM cells are a well-known human T-ALL cell line. In an example, the CEM cells in the assay are in the presence of human complement (eg, the CEM cells are mixed with human serum comprising complement proteins). When assessed against a control using non-cancerous human cells (“normal cells”) instead of CEM cells, in an example, the Hand preferentially mediates killing of said cancer cells over normal cells. The normal cells may be cancer patient cells.

Optionally, in the assay the ligand mediates the CEM cell killing with said EC₅₀ and killing of 90-100% of CEM cells.
38. The ligand of any preceding Aspect, wherein said ligand mediates ADCP killing of CEM cells in an in vitro CEM cell killing assay with an EC₅₀ in the range from 10 to 500 pM (eg, from 10 to 500 or 100 pM).

CEM cells are a well-known human T-ALL cell line. In an example, the CEM cells in the assay are in the presence of human complement (eg, the CEM cells are mixed with human serum comprising complement proteins). When assessed against a control using non-cancerous human cells (“normal cells”) instead of CEM cells, in an example, the ligand preferentially mediates killing of said cancer cells over normal cells. The normal cells may be cancer patient cells.

Optionally, in the assay the ligand mediates the CEM cell killing with said EC₅₀ and killing of 90-100% of CEM cells.

In an embodiment, said ligand mediates ADCC killing of CEM cells in an in vitro CEM cell killing assay with an EC₅₀ in the range from 10 to 500 pM (eg, from 10 to 500 or 100 pM).

CEM cells are a well-known human T-ALL cell line. In an example, the CEM cells in the assay are in the presence of human complement (eg, the CEM cells are mixed with human serum comprising complement proteins). When assessed against a control using non-cancerous human cells (“normal cells”) instead of CEM cells, in an example, the ligand preferentially mediates killing of said cancer cells over normal cells. The normal cells may be cancer patient cells.

Optionally, in the assay the ligand mediates the CEM cell killing with said EC₅₀ and killing of 90-100% of CEM cells.

In an embodiment, said ligand mediates trogocytosis killing of CEM cells in an in vitro CEM cell killing assay with an EC₅₀ in the range from 10 to 500 pM (eg, from 10 to 500 or 100 pM).

CEM cells are a well-known human T-ALL cell line. In an example, the CEM cells in the assay are in the presence of human complement (eg, the CEM cells are mixed with human serum comprising complement proteins). When assessed against a control using non-cancerous human cells (“normal cells”) instead of CEM cells, in an example, the ligand preferentially mediates killing of said cancer cells over normal cells. The normal cells may be cancer patient cells.

Optionally, in the assay the ligand mediates the CEM cell killing with said EC₅₀ and killing of 90-100% of CEM cells.
39. The ligand of any preceding Aspect, wherein the ligand specifically binds to human CD7 and Cynomolgus monkey CD7.
40. The ligand of any preceding Aspect, wherein said treatment does not produce cytokine storm syndrome in the patient.
41. The ligand of any preceding Aspect, wherein the cancer cells are CD7^{High} cells.
42. A regimen for treating or preventing a cancer in a human patient, wherein the regimen comprises performing the treatment recited in any preceding Aspect, wherein the ligand is an antibody and a first, second and third doses of the antibody are administered to the patient, wherein the doses are administered between 1 and 7 days apart, and wherein the total of said doses is from 0.1 to 100 mg/Kg of the antibody.

The invention also provides a method of treating or preventing a cancer in a human patient comprising administering to the patient a ligand of any of the Aspects.

The invention also provides a method of detecting CD7⁺ cells in a cell sample (eg, in a blood or serum sample), wherein the method comprises mixing the sample with the ligand of the invention whereby the ligand binds to CD7⁺ cells in the cell sample, and detecting or quantifying the ligand-bound cells.

Instead of, or as an example of, a “standard test”, a test may be a method used in an Example herein.

### EXAMPLES

We identified antibodies that usefully target human CD7 and would be useful to treat cancer such as T-ALL.

A particularly desirable antibody, G09, when formatted as an IgG1 comprising an E430G mutation (also referred to as “G09 E430G”) displayed human/cynomolgus CD7-cross reactivity and provided highly potent complement dependent cytotoxicity (CDC) dependent killing and potent macrophage-dependent phagocytosis activities in vitro, and robust tumour cell depletion in whole blood assays.

This data set demonstrated for an antibody comprising the G09 variable domains:
Showed potent CDC activity (EC₅₀=50-500 pM; ˜100% killing) on most non-relapsed or relapsed T-ALL cells tested in vitro
Showed potent ADCP activity on relapsed T-ALL cells in vitro
Killed T-ALL cells (˜100%) in human whole blood assay
Killed tumours in most severely immuno-compromised mice (NSG)
Reduced potency in killing of human peripheral T cells (50-75%) and NK cells (70-90%) in ex vivo assays.

### Example 1

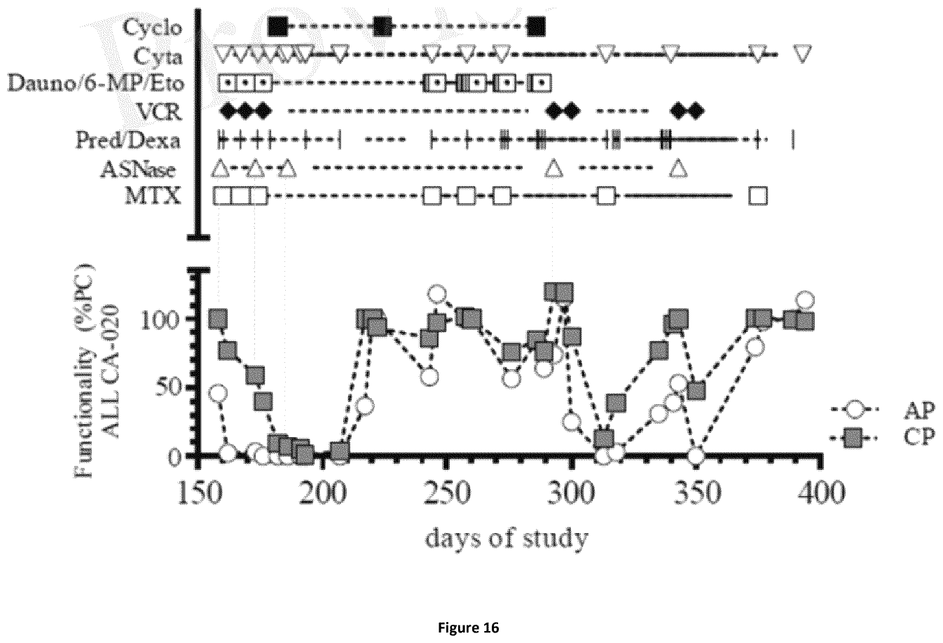

CD7 is expressed throughout the development of the T-cell lineage and is therefore expected to be expressed on all T-ALL blasts. We realised that the binding of an immunocompetent CD7-IgG1 mAb is expected to lead to a depletion of these cells based on CDC, antibody-dependent cell-mediated cytotoxicity (ADCC) and antibody-dependent cell-mediated phagocytosis (ADCP). Considering the importance to kill blasts, we wanted to have a strong depleting effect. We realised that a complicating factor is that CD7 is expressed just not on T cells but also NK cells, so there is the potential to deplete the effector cells of the ADCC response. In addition, patients with T-ALL malignancies receiving intensive chemotherapy may result in low levels of the effector cells to mediate cytotoxicity. Therefore, we realised that efficient depletion of T-ALL cells could not be achieved only by ADCC during anti-CD7 treatment. Although reductions of complement activity following chemotherapy are transient in nature, the complement activities seem effective in ALL patients in certain periods during therapy (15) (FIG. 16).

We hypothesized that by enhancing the potency of CD7-directed cytotoxicity by utilising a strategy that increases CDC activity, we could augment the efficacy of CD7-targeted therapy in patients with T-ALL malignancies.

The classical pathway of CDC relies on C1q binding to cell surface antigen-bound antibodies. The ability of human antibodies to induce CDC is isotype dependent with the order of potency IgM>>IgG3>IgG1>>IgG2=IgG4. IgM and IgG3 are difficult to manufacture making IgG1 the Fc isotype of choice for a therapeutic antibody mediating actions through CDC. Recently several point mutations, E345R, E430G and other variants in the IgG1 CH3 domain have been identified that cause immunoglobulins to form hexamer structures when bound to antigen (16). These hexamer structures can strongly enhance CDC activity by 2 to 3 orders of magnitude, inducing CDC at a level comparable to native IgM.

To evaluate these IgG1 variants, we introduced mutations into the CD7-specific benchmark antibody RFT2 (10,17) and assessed them in the CDC assay on CCRF-CEM cells (herein referred to as CEM cells, available from ATCC as ATCC® CCL-119™), a T-ALL cell line expressing CD7 using human serum as a source of complement. We found that the wild-type IgG1 version of RFT2 antibody has very limited or no killing activity on CEM cells whilst three tested IgG1 variants of the RFT2 antibody mediated potent killing of CEM cells (FIG. 1). Among them, RFT2 E345R antibody had the most potent killing activity with almost 100% maximum killing and EC₅₀ below 1 nM.

The classical pathway of CDC would be expected to be the main MOA for our lead antibody. In addition, ADCP mediated by macrophages and trogocytosis mediated by neutrophils would be evaluated. We decided that the anti-CD7 antibodies should bind to CD7 to initiate cell depletion, but they should not activate the cells. Cytokine release using human whole blood was also assessed for the lead antibody.

### Methods and Materials

### a) CDC Assay

CCRF-CEM cells (referred to as CEM cells, ATCC® CCL-119) or other T-ALL cells were suspended to 8.75×10⁴ cells/ml in the assay media (RPMI1640, 10% hiFBS). Cell suspension was plated out at 20 μl/well according to the plate map. Serial dilutions of antibodies were added at 10 μl/well according to the plate map. The human complement serum (Sigma, S1764) was reconstituted in 1 ml of ice cold water. The reconstituted serum was diluted to 1 to 3 in media and the 10 μl of diluted serum was added to each well of the plate. The plate was incubated at 37° C. and 5% CO2 for 2 hours, equilibrated to room temperature for 15 mins, and then added with 40 μl of reconstituted CellTiter-Glo® (Promega) per well. The plate was covered with an optical plate seal, agitated for 2 mins at 300 rpm on a plate shaker to ensure all the cells were lysed, and then read on the Envision plate reader using the CellTiter-Glo® 384 protocol. The luminescent signal generated from the mixtures is proportional to the amount of ATP present. The percentage of maximum killing was calculated from triplicate quadruplicate samples using the following equation:

$$\% of maximum killing = 100 - ( \frac{Assay signal value - Assay {signal}_{mediua + complement}}{Assay {signal}_{cells + complement} - Assay {signal}_{mediua + complement}} ) \times 100$$

### b) ADCP Assay

Primary human macrophages were differentiated from monocytes seeded at 2×10⁵ cells/well on 12-well plates by culturing for 7-10 days in RPMI+10% FBS supplemented with 100 ng/mL M-CSF (Peprotech) to generate monocyte-derived macrophages (MDMs). The macrophages were labelled with 2 M CellTrace™ Violet (CTV; ThermoFisher Scientific) the day before the assay and rested overnight in RPMI supplemented with 10% Ultra-Low IgG FBS (Life Technologies) and 50 ng/mL M-CSF. The CEM cell line was maintained in culture in RPMI+10% FBS. For experiments using patient-derived T-ALL xenograft cells (PDTALL-39, -46, -47 and -Ad2R), stocks were recovered from frozen on the day of the assay. Normal primary T cells were freshly isolated from macrophage donor-matched peripheral blood mononuclear cells (PBMCs) by negative selection (using the Stemcell Technologies Human T cell Isolation Kit) immediately prior to the assay. Target cells (CEM, T-ALL or normal T cells) were labelled with 2 M CellTrace™ CFSE (ThermoFisher Scientific) and resuspended in PBS.

Serial dilutions of anti-CD7 or control antibodies were prepared at 2× final concentration in 25 μL PBS. 25 μL of CFSE-labelled target cells at 4×10⁶ cells/mL (i.e. 1×10⁵ cells) were pre-opsonised with the different dilutions of anti-CD7 antibodies in PBS by incubating on ice for 0.5-1 hour. As controls, cells were mock-opsonised (no antibody) or opsonised with dilutions of appropriate human IgG1 isotype control antibody. The cells were then washed once with excess assay medium comprising RPMI+10% Ultra-Low IgG FBS (to eliminate unbound antibody) and resuspended to 5×10⁴ cells/mL in the assay medium. The medium was aspirated from the 12-well plates of CTV-labelled macrophages and 800 μL of the target cell suspension added to duplicate wells (to give 4×10⁴ target cells/well and an effector:target ratio of 5:1) and incubated for 2.5 hours at 37° C. 5% CO₂ to enable target cell phagocytosis.

Non-adherent cells were collected and combined with the adherent macrophages, which were detached using Accutase (Life Technologies) and gentle cell scraping. The cells were then washed with PBS and stained with LIVE/DEAD fixable near infra-red (IR) dead cell stain (30 min at 4° C.; ThermoFisher Scientific) before washing again with PBS and fixing for 20 min at room temp in 4% paraformaldehyde (PFA; Affymetrix). Cells were resuspended in PBS containing 2 mM EDTA for analysis. Compensation was performed using single-labelled cells and live/dead near-IR labelled ArC™ amine-reactive beads (Molecular Probes). Flow cytometry acquisition was performed on the Attune NXT Flow Cytometer using 405, 488 and 637 lasers (ThermoFisher Scientific) and the data analysed with FlowJo v10.0.8r1 (FlowJo LLC). Percent phagocytosis was calculated from duplicate samples using the following equation:

$$\% of phagocytosis = \frac{\% of cells co - labelled with C T V and C F S E}{\% of cells labelled with C F S E}$$

### c) Human Whole Blood Assay

With respect to the use of an anti-coagulant, it was decided not to use standard heparin but hirudin at a low concentration that led to successful results in the Cynomolgus study. Plasma extracted from hirudin-treated blood mediated CDC of CEM cells in vitro, whereas plasma from heparinised blood did not (data not shown). The final concentrations of 1741G09 E430G, the isotype controls, Rituximab and Ofatumumab were used as in the study.

Whole-blood cultures of three blood donors were set up in TruCulture® tubes (Myriad RBM, USA) and performed. TruCulture® (TC) tubes were filled with freshly produced media±antibodies/controls. Tubes were stored at −20° C. (<7 days) and used after thawing and thorough mixing (adjusted to room temperature). Within 60 min after phlebotomy the freshly drawn blood containing hirudin as anti-coagulants was transferred to the TruCulture® tubes and incubated for 20 h at 37° C. in a block thermostat.

At the end of the culture period, immune cells were analysed by flow cytometry. For an exact enumeration of cell counts, a defined volume of cultured whole blood was added to BD TruCount®tubes (CE, IDV). To determine the immune status 7-colour immunophenotyping panel were used (T cells CD45⁺CD3⁺; NK cells CD45⁺CD3⁻CD16⁺CD56⁺; B cells CD45⁺CD3⁻CD19⁺; monocytes CD14⁺). The cells were stained with an applied no wash protocol from the antibody manufacturer (Miltenyi Biotec; 7-Colour Immunophenotyping Kit; #130-098-456). Briefly, at the end of the incubation period, TruCulture®-tubes were centrifuged, 2 ml of the supernatant consisting of TruCulture® medium and plasma were removed, and blood cells were thoroughly resuspended. 50 μl of the resuspended immune cells were transferred into TruCount® tubes (containing a defined number of beads), detector antibodies were added and incubated for 10 min at 2-8° C. Haemolysis solution was added to eliminate erythrocytes (20 min at RT). The cells were stored at 2-8° C. before until analysis by flow cytometry. Samples were acquired on a FACSMelody™ flow cytometer (BD Bioscience).

Data were analysed by FlowJo (version 10.4.1; FlowJo, LLC). Cells per volume have been calculated according to the following equation:

$$c ( cells ) = \frac{cell count}{V ( cells )} = \frac{cell count}{bead count} \cdot c ( beads ) = \frac{cell count}{bead count} \cdot \frac{beads per tube}{V ( blood )}$$

### d) Antigen Capture ELISA for Quantitation of Serum 1741G09

The 96-well high-binding plate was coated overnight at 4° C. with 50 μl recombinant soluble human CD7 protein (Sino Biologicals, 11028-H08H) at 2 μg/ml diluted in PBS. The plate was washed 3×300 μl/well with PBS+0.1% Tween using the plate (washing buffer) washer, blocked with 200 μl PBS+1% BSA per well for at least 1 hr at room temperature, and then washed again. Serially diluted standards, samples and controls were added to the plate at 50 μl/well. After incubation for 1 hr at room temperature shaking at 300 rpm, the plate was washed 5×300 μl/well with washing buffer. The HRP conjugated anti-human IgG diluted at 1 in 30,000 in PBS+1% BSA was added at 50 μl/well. After incubation for 1 hr at room temperature, shaking at 300 rpm, the plate was washed 5×300 μl/well with washing buffer. The plate was added with TMB substrate at 50 μl/well, incubated for 30 min at room temperature protected from light, and then 50 μl/well of stop solution added. The optical density was determined within 5 min using a microplate reader set to 450 nm and correct at 640 nm. The data was imported to Softmax Pro and regressed using a 4PL curve fit with a weighting factor 1/y.

### e) Cytokine Release Assay

High binding plates were coated with 1741G09 E430G and other control antibodies at serial concentrations, and incubated at room temperature overnight, with lids off to air-dry. Plates were washed with PBS, blocked with cell culture medium by standing for 30 mins, then aspirated just before the addition of cells.

Warm medium added drop-wise to vials with cryopreserved human PBMCs and then cells were pre-incubated at high density (0.5-1×10⁷ cells/ml) at 37° C. 5% CO₂ for 24 hours. Following pre-incubation, PBMCs were seeded into 96-well polypropylene plates with 240 μl/well of culture medium (RPMI 1640, 10% FBS, 1% penicillin/streptomycin, 2 mM L-glutamine) and then incubated at 37° C. 5% CO₂ for 1 hour. PBMC cell cultures (200 μl/well, 2×10⁷ cells/well) were transferred to the immobilized test agents in the pre-prepared plates and incubated at 37° C. 5% CO₂ for 48 hours. Plates were centrifuged at 200 g for 10 mins, after which cell culture supernatants were collected and stored at −80° C. until needed for analysis.

Luminex assessment of cytokine levels in the cell culture supernatants was performed per the manufacture's protocol. Levels of induction of each cytokine were interpolated from a standard curve, using a 5-point non-linear regression analysis. The interpolated data were then normalized to the unstimulated control.

### f) Transient Protein Expression

Transient protein expression was carried out. Cultures were harvested day 12 post transfection and clarified harvests transferred to the purification team.

### g) Stable Pool Protein Expression

Stable pool protein expression was carried out.

### Discovery

### a) Lead Generation

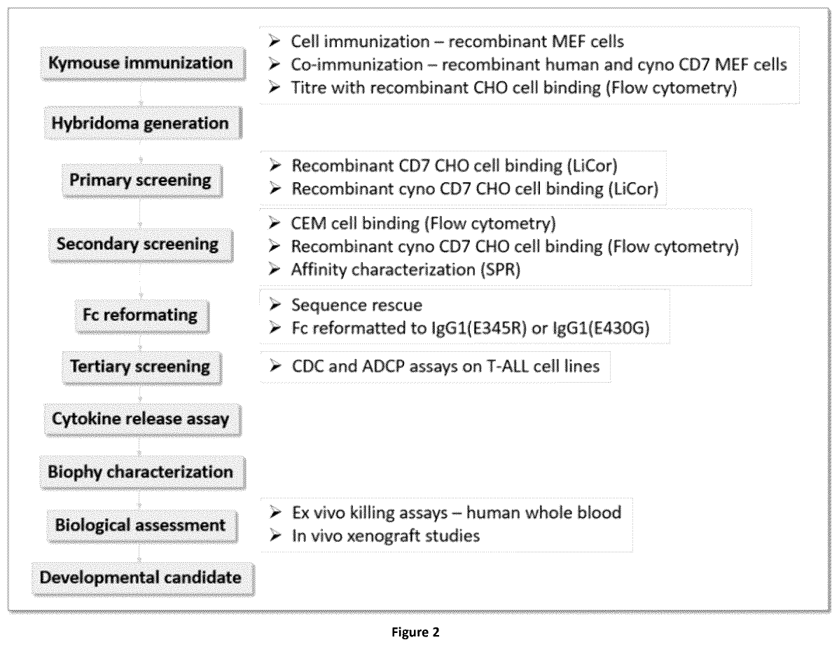

The Discovery effort for the anti-CD7 program consisted of immunizations, hybridoma generation, antibody screening, biological assessment of antibody potency, and biophysical characterization as shown in FIG. 2.

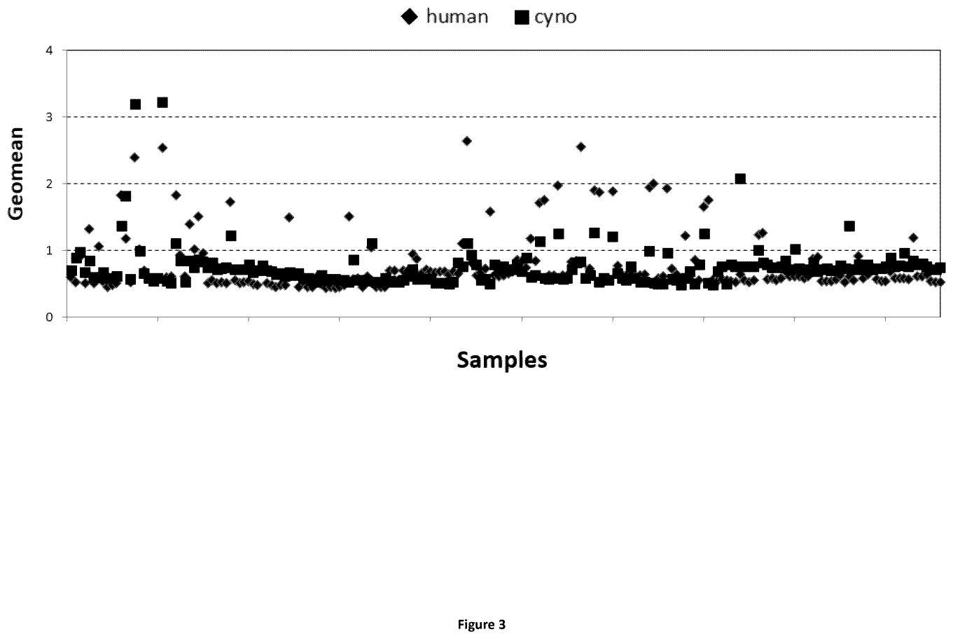

Antibodies with cross-reactivity to human and cynomolgus monkey CD7 antigens are required for assessment of the drug toxicology. Human and cynomolgus CD7 proteins only share 86% identity. To ensure this cross-reactivity, Kymice (26) were co-immunized with human and cynomolgus antigens. Titres were examined by flow cytometry analysis in which the extent of binding of polyclonal sera to human and cynomolgus CD7-expressing CHO cells was quantitated. Mice with binding titres detectable when serum was diluted by more than 10⁴ dilutions were used for hybridoma generation. There were four selection criteria for the hits from primary, secondary and tertiary in vitro screening: (i) high levels of binding to human CD7, (ii) specificity for both human and cynomolgus CD7, (iii) potent T-ALL cell depletion and (iv) an inability to cause cytokine release from T-cells. The hybridoma supernatant was used for primary and secondary screenings to identify the binders. The primary screening consisted of high throughput LiCOR-based cell binding assays to include all the potential cross-reactive binders. The secondary screening using flow cytometry was used to confirm the cross-reactivity. The outcomes are shown in FIG. 3. The human and cynomolgus CD7 binding to the CEM cells and recombinant cynomolgus CD7 CHO cells was quantitated by geometric mean fluorescence intensity (geomean) using flow cytometry. CEM cells are a human T-ALL cell line expressing endogenous CD7.

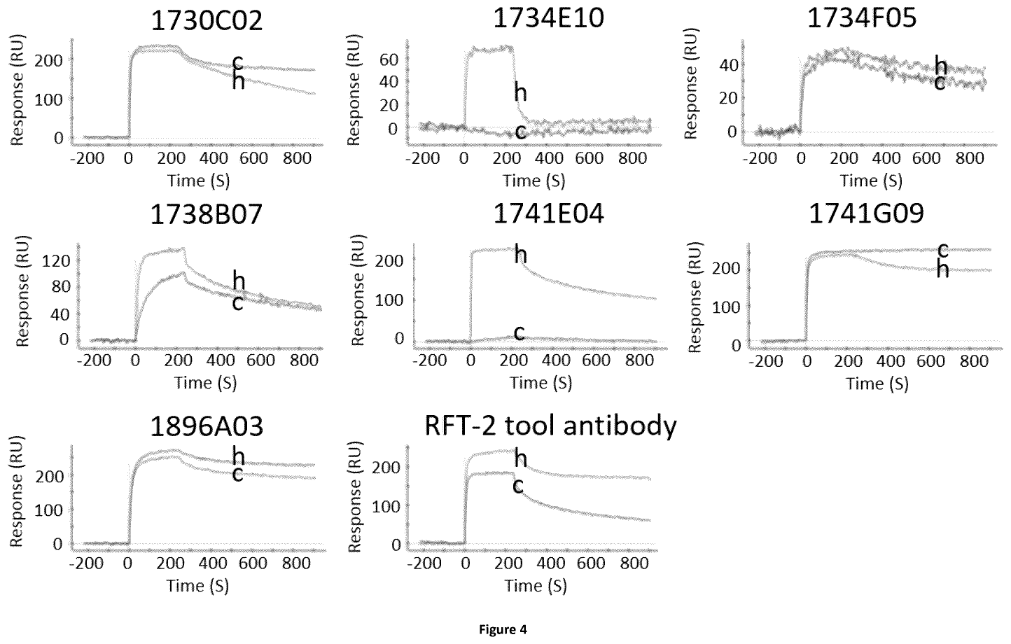

Seven hits showing strong and cross-reactive binding to human and cynomolgus CD7 were further examined for their relative binding to both antigens by measurement of SPR. Five leads (1730C2, 1734F05, 1738B07, 1741G09, 1896A03) were confirmed to show similar binding to CD7 from both species as shown in FIG. 4 and Table 1. The data were compared to the benchmark RFT2 IgG1, a chimeric antibody with mouse variable region and human constant region.

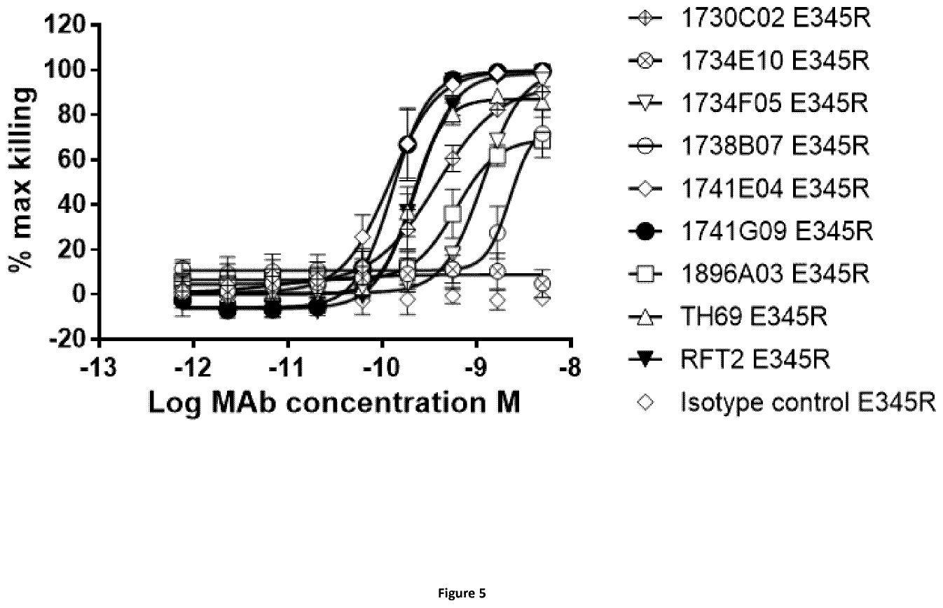

The DNA sequences of hits were retrieved from hybridoma clones and the constant region was reformatted to human IgG1 with the E345R variant. The E345R variant in the IgG1 CH3 region has been shown to enhance complement-dependent cytotoxicity (CDC) activity. As shown in FIG. 1, RFT2 IgG1 E345R but not RFT2 IgG1 could potently kills CEM cells in the CDC assays (FIG. 1). The IgG1 E345R− reformatted antibodies were expressed and tested on the CDC assay using human serum as a source of complement and CEM cells as target cells. Two leads, 1741E04 E345R and 1741G09 E345R were shown to have 100% maximum killing and potency below 200 pM in FIG. 5 and Table 2. The former only binds to human antigen, but the latter binds to both human and cynomolgus antigens. These leads were also generated with wild-type IgG1 and tested in the CDC assay. No significant killing was observed even at a concentration above 10 nM (data not shown).

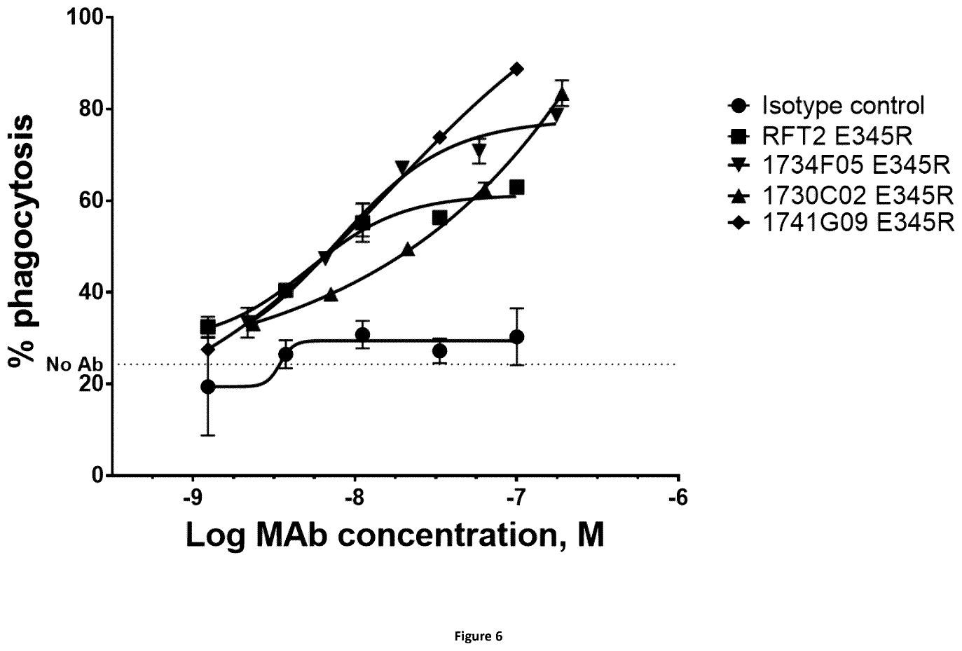

To assess whether the three leads could mediate phagocytosis by macrophages, they were tested in an ADCP assay using macrophages derived from peripheral blood monocytes as a source of phagocytes and CEM cells as target cells. 1741G09 E345R, the lead showing the best activity in the CDC assay, also showed best activity among these three leads in this assay as shown in FIG. 6.

It is generally believed that the dual staining correlates with the internalisation of target cells into the effector cells (or at least sitting inside a phagocytic cup). In the experiment, images were captured for cells at the end-point of the phagocytosis assay using the Amnis Imagestream (Merck Millipore), and were analysed using software tools that allow the discrimination of internalized from extracellular-bound particles to demonstrate phagocytosis of target cells. Similar techniques have been described elsewhere for this purpose (27). Furthermore, the use of Accutase for cell harvesting by enzymatic detachment at the end-point of the assay would likely disrupt inter-cellular contacts that could be mis-interpreted as phagocytosis (internalization) events.

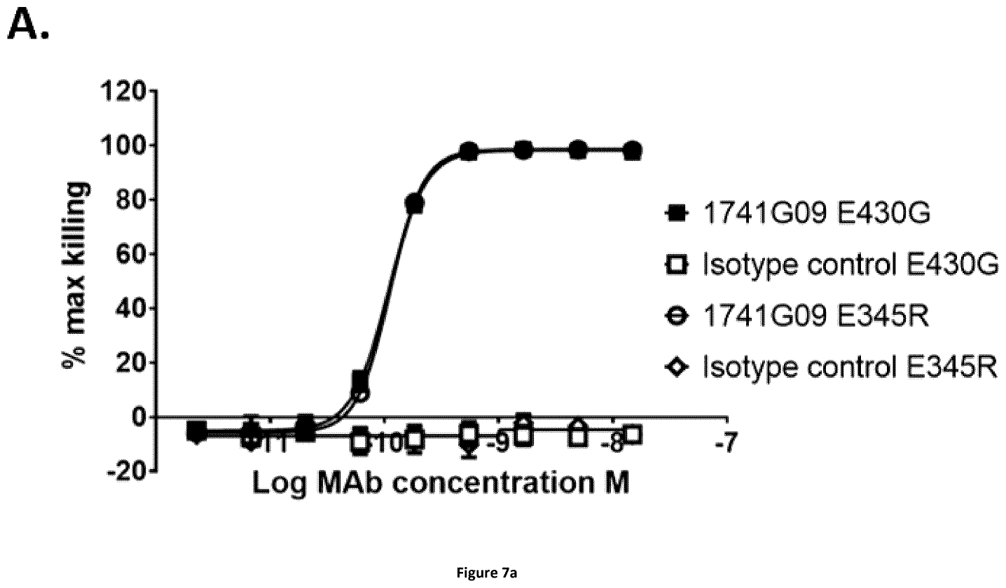

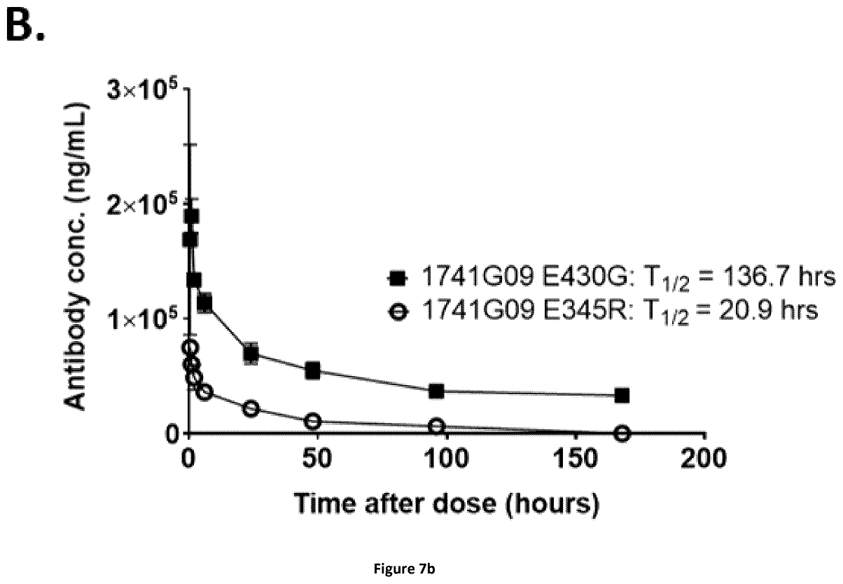

During the progress of our screening, there was another variant, IgG1 E430G that was also reported to enhance the CDC activity (28). The 1741G09 E430G variant has similar killing potency to the 1741G09 E345R variant in our test (FIG. 7A). The 1741G09 E430 has a half-life 136.7 hours in NSG mice (FIG. 7B), which is within in the normal range of human IgG1 in such mice. However, 1741G09 E345R had a much shorter half-life of only 20.9 hours, potentially because this variant has a higher tendency to aggregate. Based on this observation, we replaced 1741G09 E345R with 1741G09 E430G as a lead molecule and further assessed the CDC and ADCP activities of this molecule.

### b) Lead In Vitro Profile

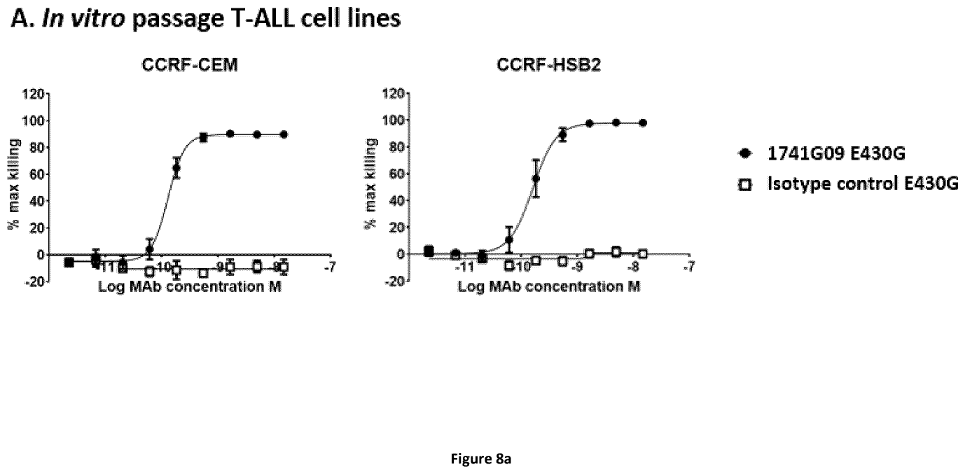

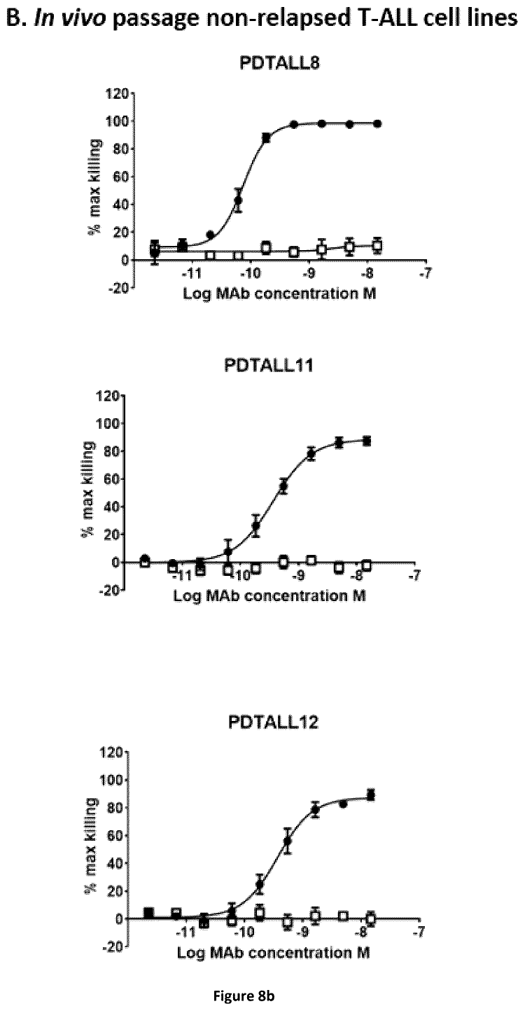

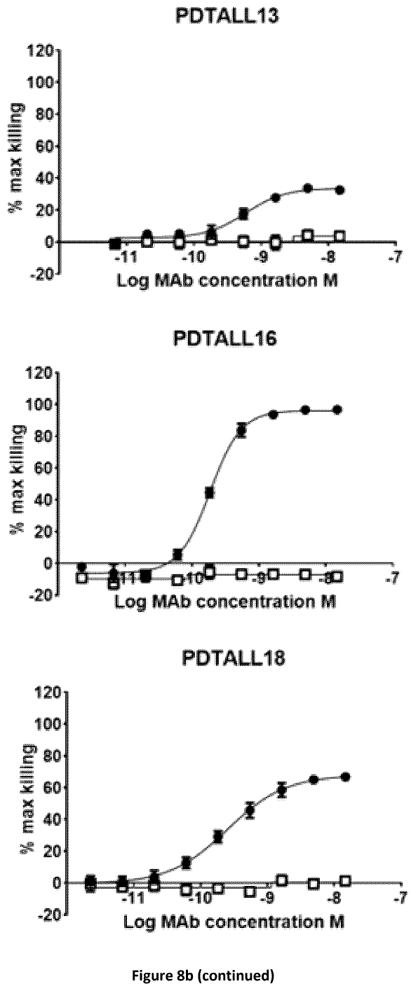

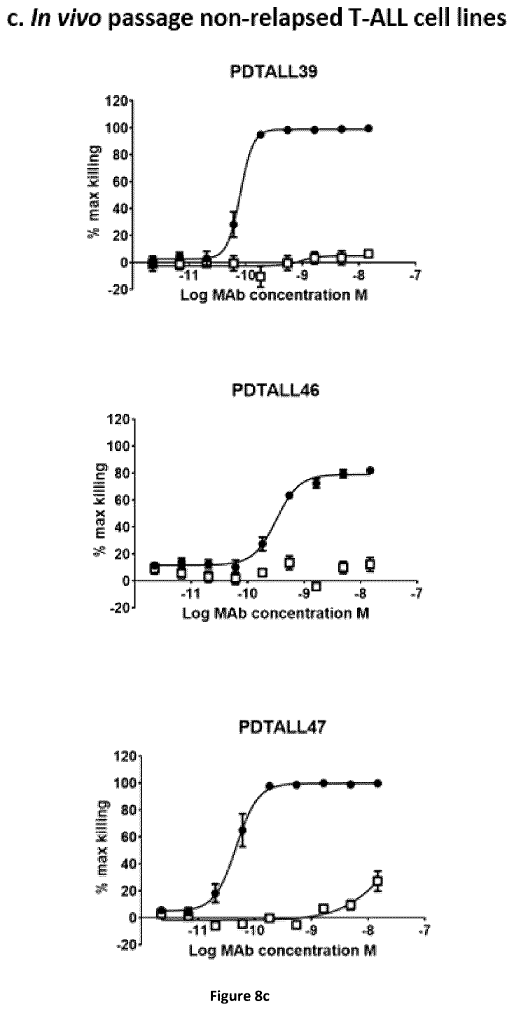

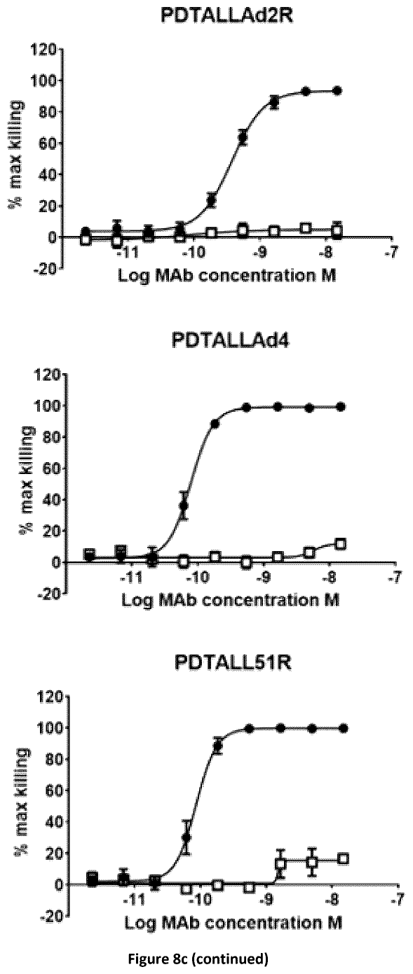

The antibody discovery campaign was based on binding, cell-depletion assays and half-life of molecules identified a lead antibody, 1741G09 E430G. To assess its killing activity on different T-ALL cells, the lead was tested in the CDC assays on 14 different T-ALL cell lines including two commercial in vitro passage cell lines (CEM and HSB2), six in vivo passage non-relapsed cell lines (PDTALL8, PDTALL11, PDTALL12, PDTALL13, PDTALL16 and PDTALL18), and six in vivo passage relapsed cell lines (PDTALL39, PDTALL46, PDTALL47, PDTALL51R, PDTALLAd2R and PDTALLAd4) (29). The clinical and genetic profiles of these cell lines are shown in Table 7. 1741G09 E430G was demonstrated to effectively kill the majority of cell lines (11 cell lines out of 14) (FIGS. 8A-8C), with a potency around 50-500 pM (Table 3).

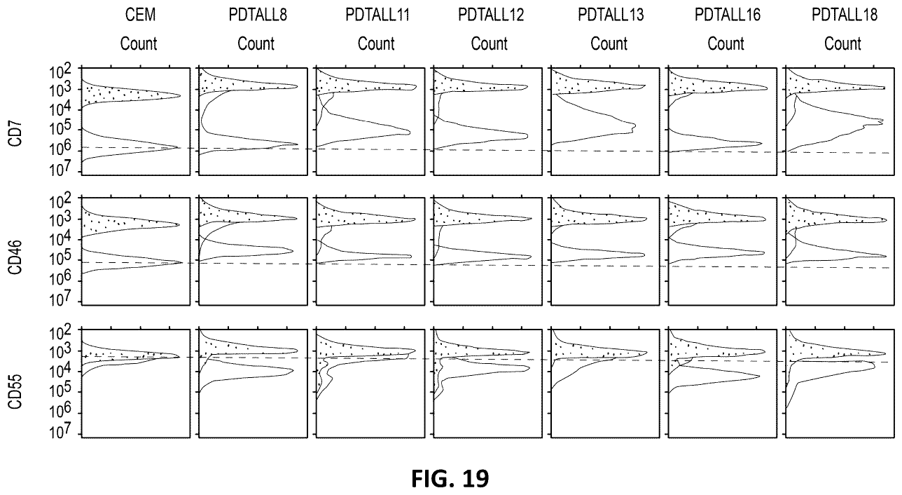

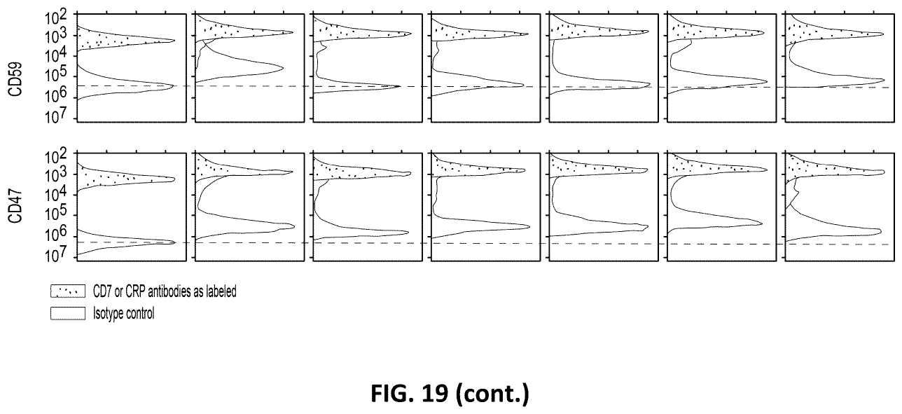

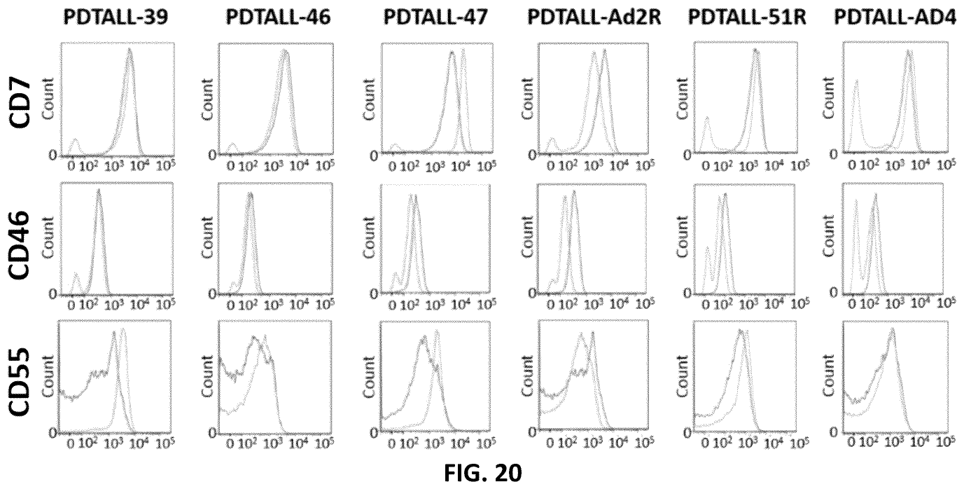

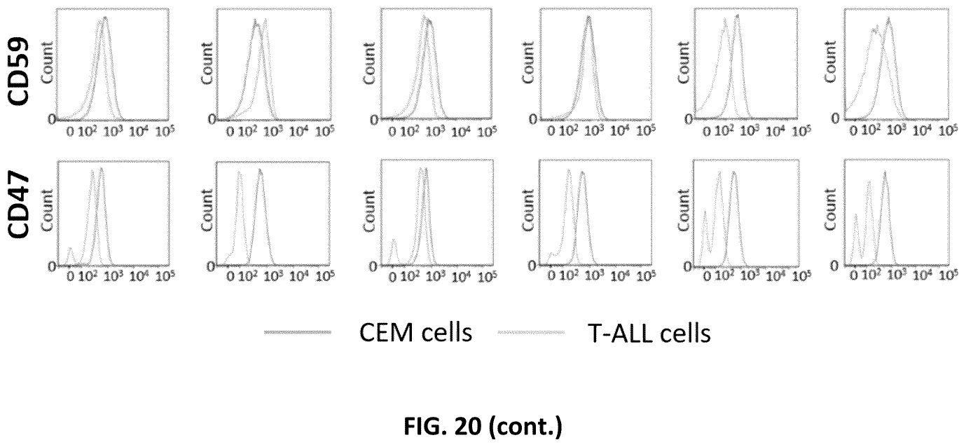

In the assays conducted, the potency and maximum killing were correlated to cell surface CD7 expression (FIGS. 19 and 20). The cell lines could be ranked based on the CD7 expression level on the cell surface as shown in Table 3. Reflecting the expression level, PDTALL47, HSB, PDTALLAd4, PDTALL51R, PDTALL39, PDTALL8, CEM and PDTALL16 with the high or intermediate CD7 expression levels (relatively to CEM cells) had highest maximum killing, close to 100%, and EC₅₀ values from 50 pM to 200 pM; PDATLLAd2R, PDATALL12 and PDTALL11 with the intermediate to low expression levels had maximum killing close to 90% and EC₅₀ below 400 pM; PDTALL13 and PDTALL18 with much low expression levels had maximum killing below 70% and EC₅₀ below 600 pM (Table 3). In consideration of the CD7 expression level, PDTALL46 has a higher level than PDTALLAd2R. Both cell lines had similar EC₅₀ close to 350 pM, but PDTALL46 had lower maximum killing than PDTALLAd2R (78% vs 93%) possibly because the former had higher expression levels of complement-regulatory proteins (CRPs: CD46, CD52 and CD59) than the latter (FIG. 20). CRPs have been reported to function as antagonists against complement activity on cell surface. In summary, these results suggest that the antibody potency of CDC activity is majorly dependent on the target antigen expression on the cell surface and can be regulated by CRPs.

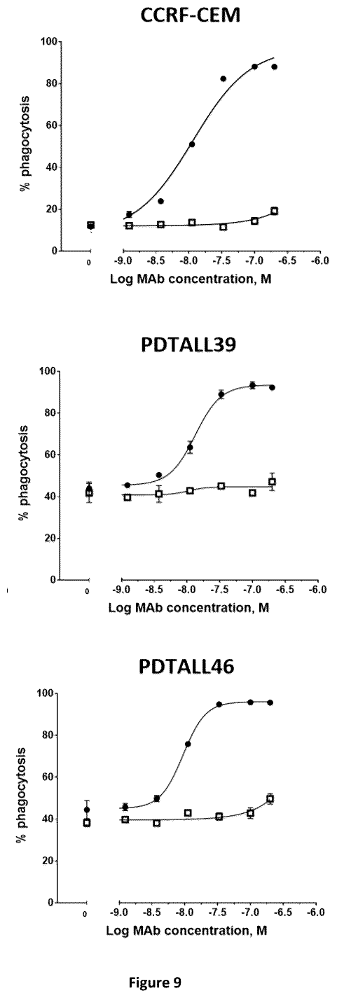

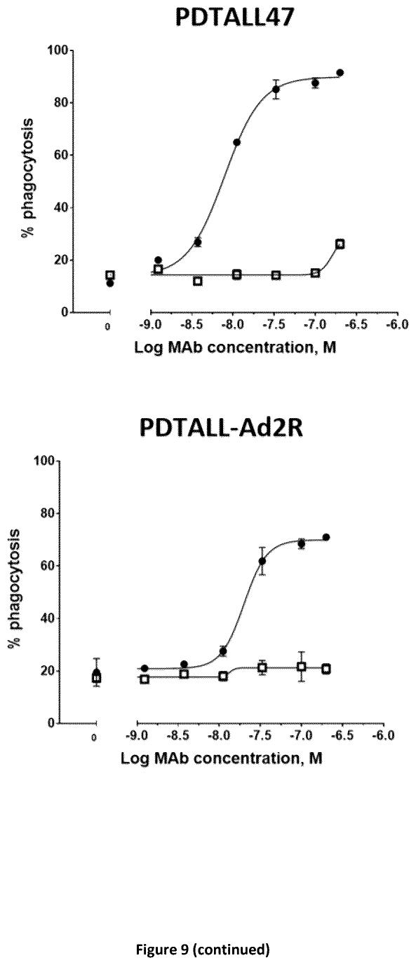

The lead was also tested in the ADCP assay using different T-ALL cells as shown in FIG. 9. Human peripheral monocyte-derived macrophages were differentiated and used to assess the ADCP activity of the 1741G09 E430G molecule on T-ALL cell lines. Among five cell lines tested, four showed close to 100% phagocytosis by macrophages. 1741G09 E430G only depleted 80% of the relapsed cell line PDTALL46 cells in the CDC assay. However, it mediated almost 100% phagocytosis of the same cells in the ADCP assay. On the other hand, 1741G09 E430G only mediated 75% of the other relapsed cell line PDTALLAd2R cells in the ADCP assay. However, 1741G09 E430G depleted close to 95% of the same cells in the CDC assay. These data support the theory that multiple MOAs could complement each other and mediate effective killing of T-ALL cells.

### c) Cytokine Release Profile

As CD7 is expressed on peripheral T and NK cells, antibody ligation may lead to activation of these cells. It has been reported that anti-CD7 mAbs could be mitogenic, increasing calcium flux and augmenting IL-2 production (30). However, anti-CD7 mAb, RFT-2 did not raise any significant concerns of cytokine storm in a previous clinical trial for the treatment of renal transplantation (10).

To evaluate the ability of the 1741G09 E430G antibody to stimulate human peripheral blood mononuclear cells (PBMCs), we tested the molecule for its effect on cytokine release from PBMC, following immobilization by air-drying.

The 1741G09 E430G was evaluated in PBMCs from five individual donors, as measured by the release of specific cytokines and chemokines. Corresponding isotype controls were used to monitor non-specific activation of the PBMC cultures. Super-agonistic anti-CD28 and anti-CD3 (OKT3) antibodies were used as positive controls.

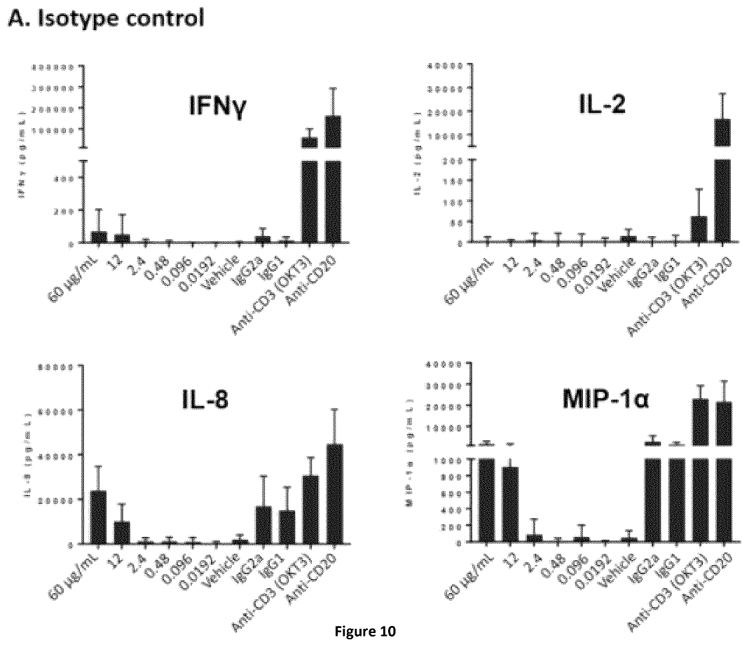

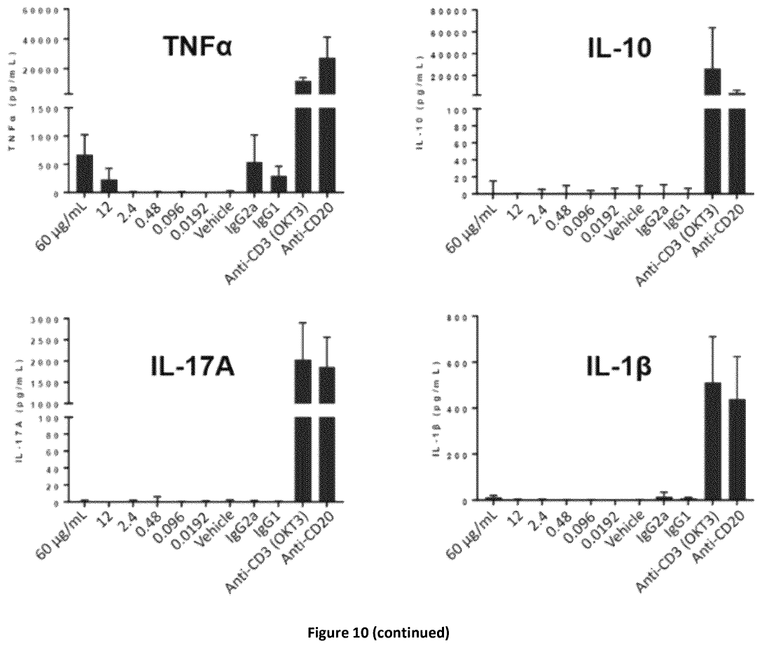

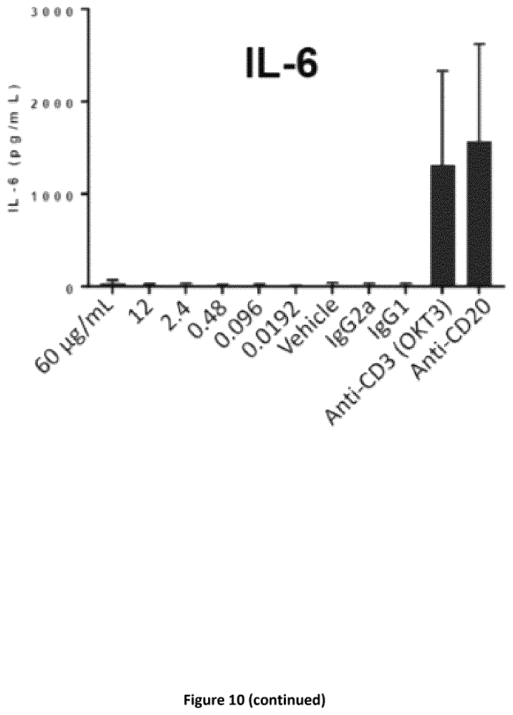

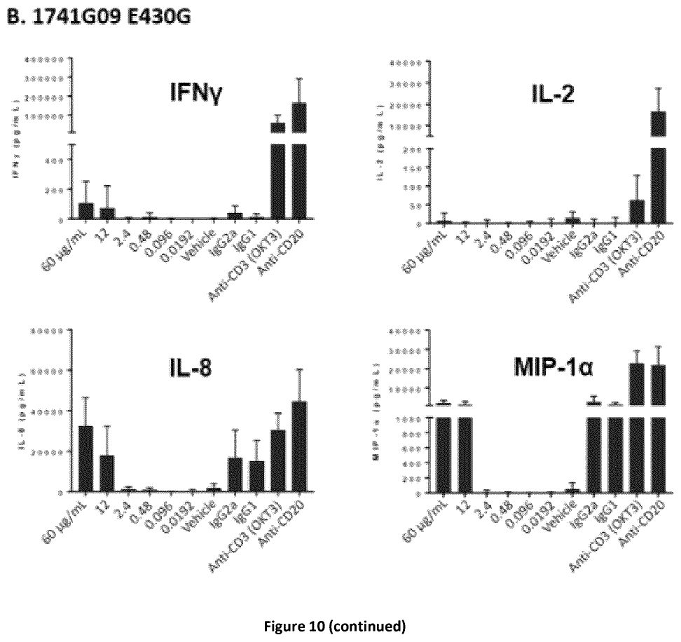

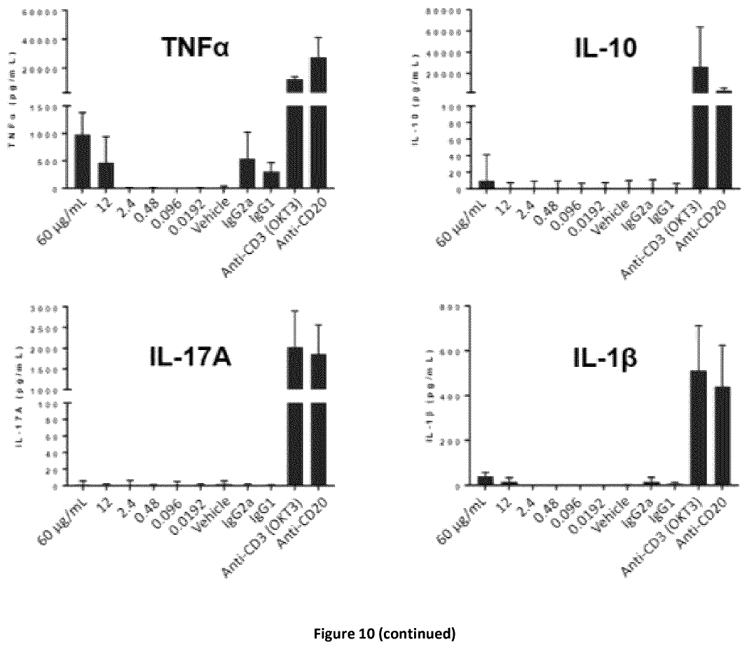

Among the cytokine panels, six cytokines were slightly increased in the samples treated with 1741G09 E430G when compared to ones treated with IgG1 at the top test concentration (60 μg/ml) (IL8, 2.8×; MIP-1α, 2.9×; TNFα, 4.2×; IL1β, 4.6×; IL6, 1.7×). The IgG1 E430G isotype control also slightly increased these cytokine release, suggesting that the increases may not be CD7-specific. The positive controls, anti-CD3 (clone OKT3) and super agonistic anti-CD28, induced characteristic cytokine profiles from all donors, confirming that the assay had performed as expected, with much higher general induction than any test article or isotype control (FIG. 10 & Table 4).

### Discovery Campaign Summary

Seven leads were selected from primary and secondary cell binding screening. Five were confirmed with cross-reactivity to human and cynomolgus CD7 proteins via measurement of binding to soluble CD7 by SPR. These five leads were reformatted to human IgG1 E345R Fc and tested in the CDC and ADCP assays on CEM cells. The 1741G09 E345R antibody was selected as the lead molecule as it was with the most potent CDC and ADCP activities. The lead antibody was further reformatted to IgG1 E430G Fc in consideration of better half-life of this variant in vivo. The potent CDC activities of the 1741G9 E430G antibody were also demonstrated in different T-ALL cell lines with maximum killing of 100% for the majority of cell lines and strong potency (EC₅₀=50-500 pM). The 1741G09 E430G mAb did not significantly increase the cytokine release from PBMC, when it was compared to super agonists, anti-CD3 or anti-CD28.

### c) Biological Assessment

### In Vitro CDC Assay on PBMC T and NK Cells

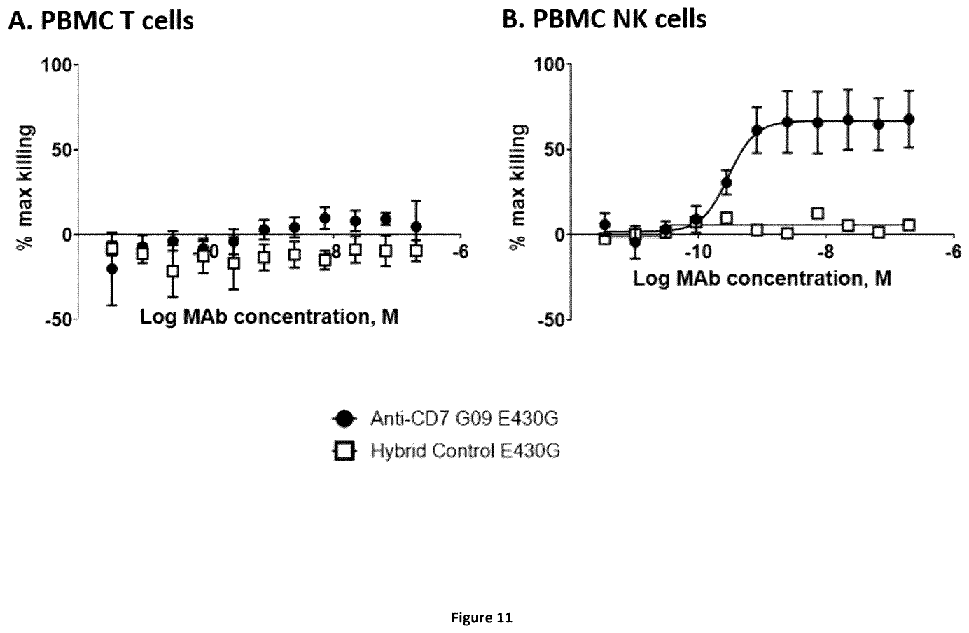

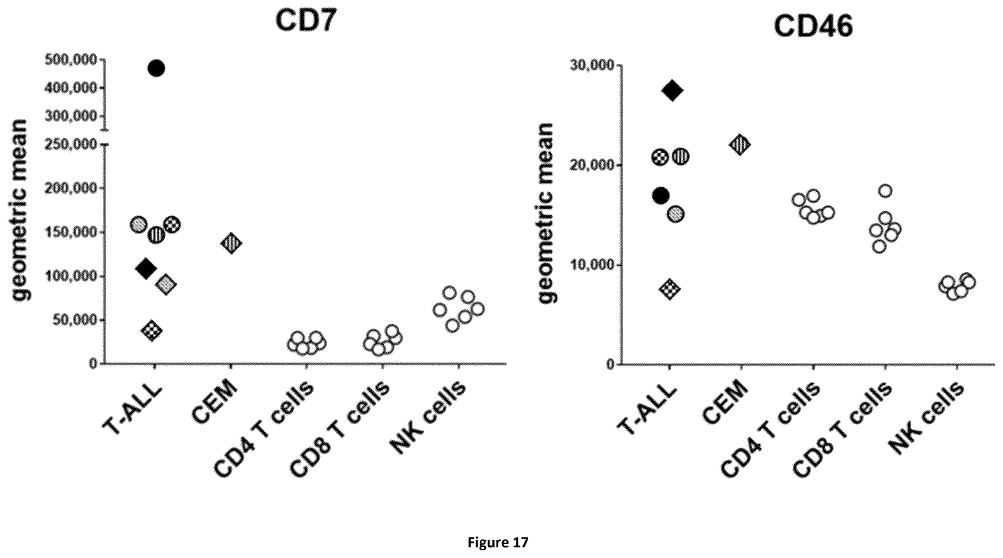

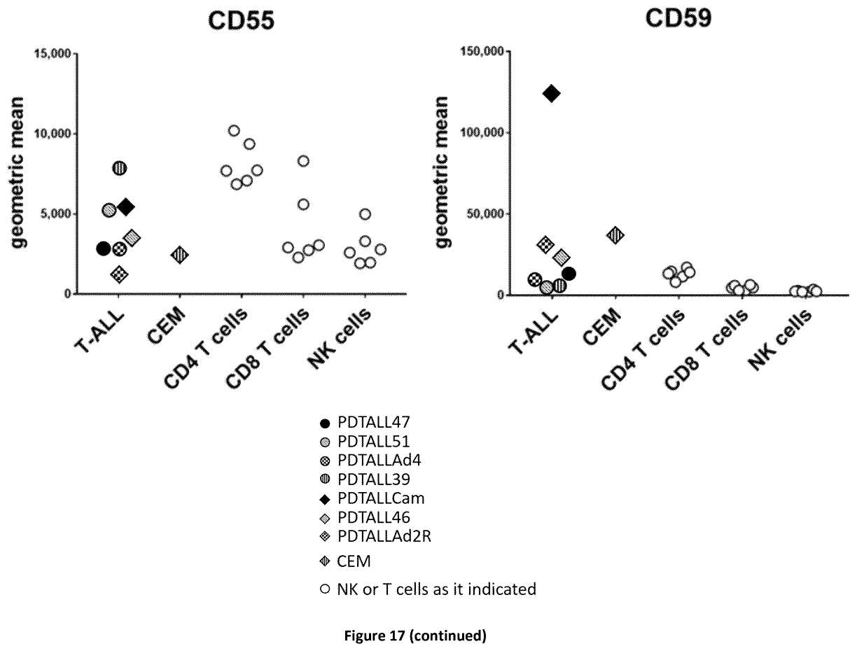

As peripheral T and NK cells express CD7, they are susceptible to depletion mediated by the lead molecule. Peripheral T and NK cells were isolated respectively from two different donors and used to assess the CDC activity of 1741G09 E430G. While no significant T cell depletion was detected (FIG. 11.A), a maximum of 65% of NK cells were depleted with EC₅₀ close to 300 pM (FIG. 11.B) within the range of EC₅₀ values observed for the T-ALL cell depletion (Table 2). Notably, NK cells have a higher expression of CD7 and lower expression of CRPs than T cells (FIG. 17).

### Ex Vivo Human Whole Blood Assay

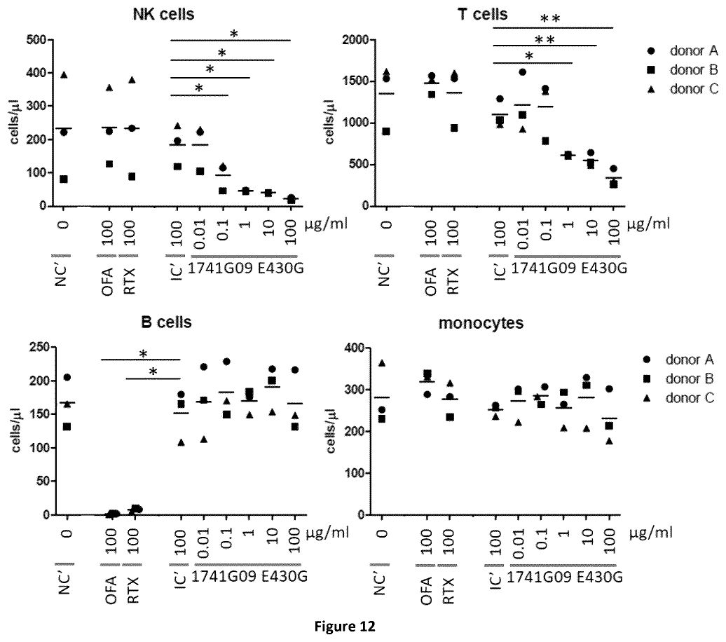

To assess if 1741G09 could deplete normal peripheral T and NK cells in a more physiological context, we used a TruCulture®-based assay to evaluate the effects of the antibody on the human whole blood status. The 1741G09 E430G antibody was used in concentrations ranging from 0.01 to 100 μg/ml and compared to isotype control (100 μg/ml). Two positive controls, rituximab and ofatumumab (100 μg/ml) were also included. Whole blood and antibodies were incubated for 20 hours prior to assessment of cell depletion. Three human donors were used in the assays, referred to as A, B and C. Numbers of cell subset per μl of sample were determined by flow cytometry. Results are shown in FIG. 12. Assay were performed by Hotscreen.

The isotype control treated cultures showed very similar cell counts compared to the negative control across the four cell types measured; B, T, NK cells and monocytes. Ofatumumab and Rituximab are therapeutic antibodies targeting B cells, and were included as positive controls for specific cell depletion. Indeed, B cells were almost completely eliminated throughout the culture. A concentration-dependent depletion of T cells and NK cells by 1741G09 E430G were observed in all three donors. The numbers of T and NK cells were reduced as compared to the negative (no antibody) or isotype control. The 1741G09 E430G antibody depleted NK cells up to 90% and T cells up to 75%. The antibody was more potent for NK depletion than T cell depletion. At the concentration of 0.1 μg/ml (≈0.67 nM) of 1741G09 E430G, NK but not T cells were significantly depleted.

The reduction of T cells appeared to have no effect on the ratio between CD4⁺ and CD8⁺ T cells (data not shown). There was no significant change of B cell and monocyte counts, indicating the specificity of 1741G09 E430G for its cell depletion activity.

We observed up to 65% depletion of NK cells but no significant T cell depletion in the CDC assay using human serum and isolated NK or T cells (FIG. 11). The higher maximum depletion rates (90% of NK cells and 75% of T cells) obtained from the human whole blood assays (FIG. 12) suggest that there are other effector cell components involved in the cell depletion in this assay.

Ex Vivo Human Whole Blood Assay Spiked with T-ALL Cells

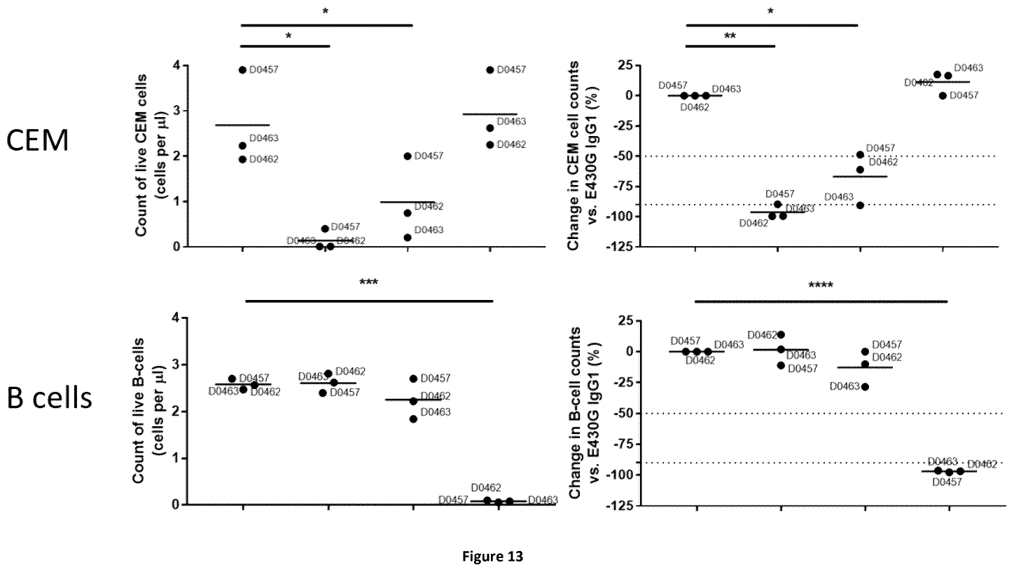

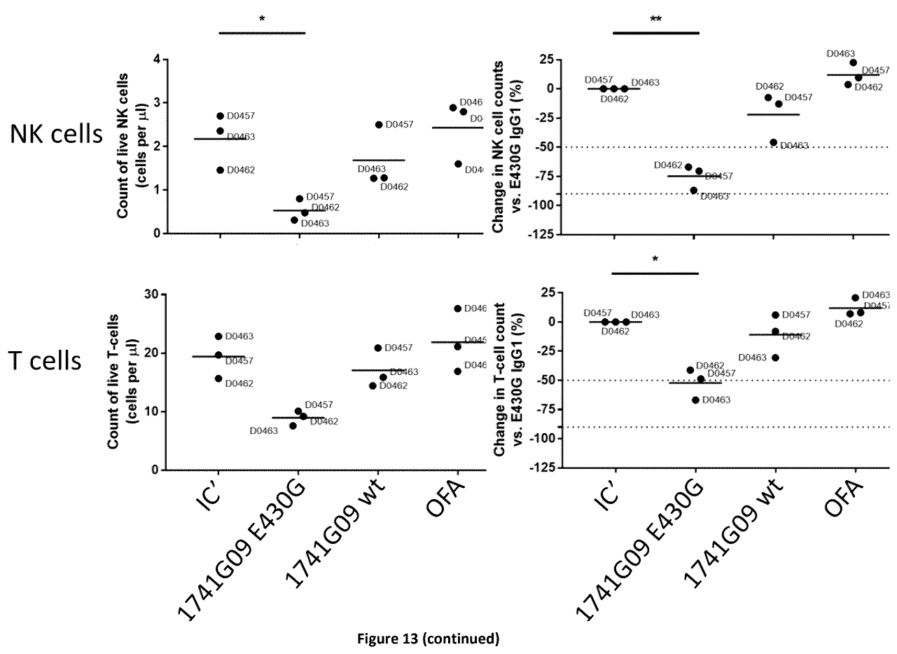

Tumour cell depletion should ideally be assessed in the blood samples from T-ALL. However, it is difficult to obtain such samples as patients are rare and primary T-ALL cells are difficult to keep in culture. To circumvent this issue, we spiked the blood samples from the healthy donor with the T-ALL cell-line, CEM cells. The survival of CEM cells as well as the survival of the NK, T- and B-cells from the healthy donor were assessed in presence of 10 μg/ml (≈67 nM) of 1741G09 IgG1 E430G, 1741G09 IgG1 wild type, E430G IgG1 isotype control or Ofatumumab. The high concentration of antibodies was used to ensure maximum killing was achieved. Results are shown in FIG. 13.

1741G09 E430G significantly decreased CEM cell counts in spiked whole blood samples. The 1741G09 wt antibody was included as a comparison to the CDC-enhanced version. Unlike the 1741G09 E430G antibody, the wt Fc version of 1741G09 has been shown not to significantly deplete CEM cells in the CDC assays (data not shown). In the whole blood assay, both wt and E430G versions of 1741G09 significantly decreased the counts of CEM cells but the efficacy of the E430G version was much greater (mean depletion percentage 96.2% vs 66.8%). The depletion of CEM cells by the 1741G09 wt antibody suggests that other effector components except CDC are involved in the process. As expected, ofatumumab strongly decreased B-cell counts without impacting CEM, NK or T-cell counts.

The NK and T cell counts were also significantly decreased by 1741G09 E430G but not 1741G09 wild type. The depletion of NK cells (75%) and T cells (52%) was not as high as the depletion of CEM cells (96%). This data confirmed that the 1741G09 E430G antibody impact is linked to the CD7 expression level of the target cells (CEM cell>NK cell>T cell, FIG. 21), and its impact on cell depletion is greater than the wild type Fc version.

### Impact of Anti-C5a Antibody on Lead-Induced Cell Depletion

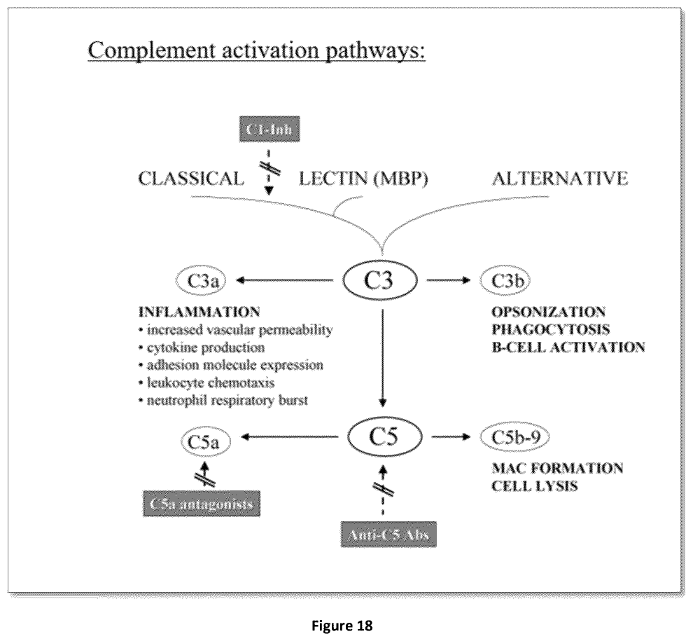

To better understand the lead's mode of action, we assessed the effect of an anti-complement 5a (C5a) antibody on the cell depletion induced by 1741G09 E430G, 1741G09 wild type and Ofatumumab. The complement pathways can be divided into the activation pathway and the lytic pathway (FIG. 18) (31). C5, via C3, can be cleaved into C5a (activation pathway) and C5b (lytic pathway). C5a has chemotactic and anaphylatoxic properties and plays an essential part of the innate immune response (smooth muscle contraction, vascular permeability, degranulation of mast cells and basophils, directed migration of neutrophils, eosinophils, basophils and monocytes). The antibody used in the present assay is capable of inhibiting the binding of C5a to the C5a receptor, without blocking the cleavage of C5 (32).

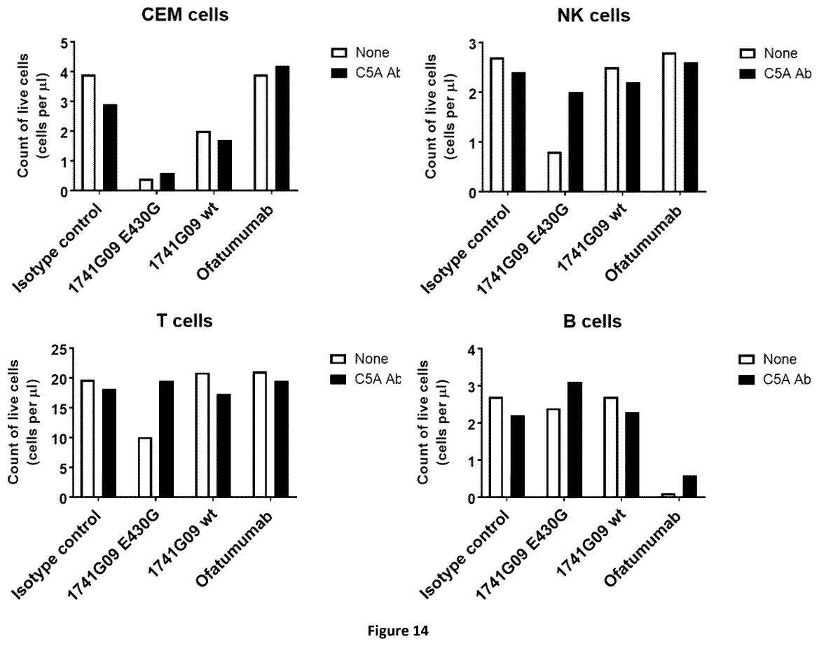

Both wt and E430G versions of 1741G09 decreased the counts of CEM cells (by 49% and 90% respectively) (FIG. 14). Addition of anti-C5a antibody did not impact CEM cell depletion induced by either version. Although CEM cell depletion was not blocked by anti-C5a, the MOAs behind these two versions of 1741G09 are expected to be quite different. The lack of inhibition of CEM depletion by 1741G09 E430G supports the hypothesis that this antibody acts via the lytic pathway. It is unlikely that E430G-driven depletion is attributed to NK cell-mediated ADCC because the NK cells would quickly get depleted through CDC once 1741G09 E430G is added. In contrast, the wt version does not deplete either NK or CEM cells via CDC (data not shown). Thus, depletion of CEM cells by the wt version is likely via NK cells-mediated ADCC, macrophage-mediated phagocytosis or neutrophil-mediated trogocytosis, none of which appear to be inhibited by the anti-C5a antibody in this experimental set-up.

The 1741G09 E430G antibody decreased the counts of NK and T-cells (by 70 and 49% respectively). In contrast to CEM cells, addition of anti-C5a reverted 1741G09 E430G-induced T-cell decrease into the similar cell counts to isotype control (FIG. 14), suggesting that the depletion of T cells by 1741G09 E430G is not attributed to the classical or lytic pathway but to the inflammatory pathway of the complement activation. This is consistent to the result that 1741G09 E430G did not significantly deplete PBMC T cells in the CDC assay. Addition of anti-C5a also partially reverted 1741G09 E430G-induced NK-cell decrease, suggesting that this molecule depletes NK cells via both activation and lytic pathways. Ofatumumab effects may be partially reversed by anti-C5a; however, it should be noted that this experiment was performed in one donor only. Incubation of whole blood with 1741G09 in the presence of a lytic pathway inhibitor, such as eculizumab, would confirm the requirement for this pathway.

### In Vivo Xenograft Study

Anti-CD7 antibodies have previously demonstrated efficacy in the xenograft model (33). To assess the efficacy of the 1741G09 E430G antibody, we tested the antibody in a paediatric relapsed PDX T-ALL xenograft model.

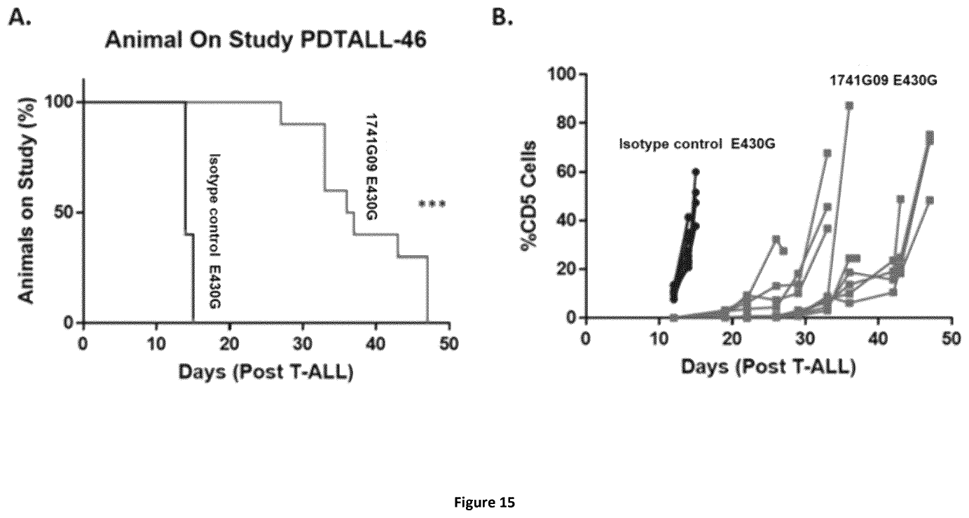

The NSG mice were dosed at 10 mg/Kg three times a week from day 3 until the end of the study following injection with 5×10⁶ PDTALL46 cells at day 0. Blood draws were taken at serial time points to assess human CD5 expression levels in the blood by flow cytometry, as an antibody to anti-CD7 that does not compete with 1741G09 was not identified. The Kaplan-Meier plot demonstrated a significant increase in survival time in the group treated with 1741G09 E345R compared to the group with isotype control (FIG. 15). As NSG mice are severely immunodeficient they lack T, B and NK cells, and complement activity and macrophages are also defective. Neutrophil-mediated trogocytosis (34) in the presence of 1741G09 E430G is likely to have mediated the observed depletion in this mouse model.

### Developability

As part of the developability assessment of the candidate 1741G09 E430G three pre-CMC studies have been performed: early formulation screening, accelerated/real time studies and forced degradation studies.

In early formulation screening the candidate was dissolved into 12+ different platform formulation buffers followed by colloidal and conformation stability assessment (Tₘ and Tagg determination, by intrinsic fluorescence and SLS respectively). Two platform buffers demonstrated suitable stability and were short-listed for the accelerated and real time studies: the candidate was prepared at 1 mg/ml in each of the two formulation buffers, or PBS as additional control and incubated at 5° C., 25° C. and 37° C. for two weeks. The experiment was performed in triplicate for each condition. The following quality attributes were measured: aggregates by SEC-HPLC and DLS, fragments by SDS-PAGE, activity by CDC functional assay. No significant changes were observed in any of the quality attributes or activity after a two-week incubation at any tested condition. Additionally, the candidate was subjected to freeze/thaw stress and no changes in the same quality attributes were observed after 3 cycles of storage at −70° C. for at least 18 hours followed by thawing at room temperature for 3 hours.

Finally, forced degradation studies were performed on the candidate at 1 mg/ml in PBS: forced deamidation (72-hour incubation at 37° C. in 1% ammonium bicarbonate), forced oxidation (24 hour incubation at 25° C. in 0.03%, 0.003% and 0.0003% H₂O₂) and acidic hold (3-hour incubation at 25° C. and pH 2.8). Activity by CDC functional assay was tested in duplicate before and after the stress conditions. No significant changes were observed after forced deamidation, acid hold and forced oxidation at the lower and intermediate level of H₂O₂, while the oxidation at the higher H₂O₂ concentration showed a reduction in activity by 87% and 4-fold increase in EC₅₀.

In line with in-silico predictions, experimental data indicated low risk from a developability perspective: the candidate can be purified through platform protein A process with suitable product quality (aggregates lower than 1%, nominal concentration 10 mg/ml into PBS pH 7.4 as platform buffer). The early formulation screening demonstrated platform formulation buffers can further improve colloidal and conformation stability, while accelerated and real time conditions after two weeks do not affect product quality or activity. The force degradation tests highlighted overall a low risk with no effect due to freeze/thaw, acidic hold, deamidation; the risk is considered low/medium for oxidation and may be managed through formulation by addition of sacrificial antioxidant excipients.

Overall, the candidate presents low risks from a pre-CMC standpoint based on the computational and experimental dataset presented here.

### Lead Molecule Expression

The lead molecule in the CD7 project, 1741G09 HuIgG1 E430G C-term Lysine clipped, Phe variant of light chain (1741G09 E430G), has been expressed in both a transient and stable pool expression system.

1741G09 E430G was expressed at three different scales in the transient system, 30 ml, 200 ml and 2 L. There was no significant difference in cell growth or viability compared to cultures expressing a control antibody. The expression levels were the same in all scales at day 5 post transfection, whilst expression at day 12 diverged, with expression levels of >600 mg/L at the 30 ml and 2 L scale. Expression plateaued at the 200 ml scale. This difference in expression yield at different scales is not uncommon.

1741G09 E430G expression yield at the 30 ml and 2 L scale is greater than the standard, high expressing control antibody expressed alongside all molecules (FIG. 18). This is a good initial indicator that 1741G09 E430G can be expressed at appropriate levels. However, it is important to note that, although anecdotal evidence suggests that if a molecule is high expressing in the transient system then it is likely to yield a high expressing stable cell line, the predictability of stable outcomes from the transient system has not been fully evaluated.

Three cell pools stably expressing 1741G09 E430G have been generated. The average expression of these pools on day 13 of a fed batch overgrow was 385 mg/L. 1741G09 E430G stable pool expression was 43% of that observed with high expressing control antibody pools generated in parallel. Pools likely contain a mixture of high- and low-expressing clones, and so selection of an appropriate clone would probably give us a good yield. Indeed, for an unrelated molecule minipools were generated from a pool that expressed ˜50% of control, and >5× improvement in expression achieved.

### REFERENCES

1. Ware R E, Haynes B F. T cell CD7 mRNA expression is regulated by both transcriptional and post-transcriptional mechanisms. Int Immunol. 1993 February; 5(2):179-87.
2. Barcena A, Muench M O, Galy A H, Cupp J, Roncarolo M G, Phillips J H, et al. Phenotypic and functional analysis of T-cell precursors in the human fetal liver and thymus: CD7 expression in the early stages of T- and myeloid-cell development. Blood. 1993 Dec. 1; 82(11):3401-14.
3. Hao Q-L, George A A, Zhu J, Barsky L, Zielinska E, Wang X, et al. Human intrathymic lineage commitment is marked by differential CD7 expression: identification of CD7− lympho-myeloid thymic progenitors. Blood. 2008 Feb. 1; 111(3):1318-26.
4. Reinhold U, Abken H. CD4+CD7− T cells: a separate subpopulation of memory T cells? J Clin Immunol. 1997 July; 17(4):265-71.
5. Ware R E, Hart M K, Haynes B F. Induction of T cell CD7 gene transcription by nonmitogenic ionomycin-induced transmembrane calcium flux. J Immunol Baltim Md 1950. 1991 Oct. 15; 147(8):2787-94.
6. Rabinowich H, Lin W C, Herberman R B, Whiteside T L. Signaling via CD7 molecules on human N K cells. Induction of tyrosine phosphorylation and beta 1 integrin-mediated adhesion to fibronectin. J Immunol Baltim Md 1950. 1994 Oct. 15; 153(8):3504-13.
7. Lee D M, Staats H F, Sundy J S, Patel D D, Sempowski G D, Scearce R M, et al. Immunologic characterization of CD7-deficient mice. J Immunol Baltim Md 1950. 1998 Jun. 15; 160(12):5749-56.
8. Sempowski G D, Lee D M, Scearce R M, Patel D D, Haynes B F. Resistance of CD7-deficient mice to lipopolysaccharide-induced shock syndromes. J Exp Med. 1999 Mar. 15; 189(6):1011-6.
9. Carriere D, Arcier J M, Derocq J M, Fontaine C, Richer G. Antigenic modulation induced by four monoclonal antibodies adsorbed on gold particles (specificity anti-CD4, anti-CD5, anti-CD7, and anti-150-kDa antigen): Relationship between modulation and cytotoxic activity of immunotoxins. Exp Cell Res. 1989 May; 182(1):114-28.
10. Sharma L C, Muirhead N, Lazarovits A I. Human mouse chimeric CD7 monoclonal antibody (SDZCHH380) for the prophylaxis of kidney transplant rejection: analysis beyond 4 years. Transplant Proc. 1997 March; 29(1-2):323-4.
11. Rappl G, Abken H, Muche J M, Sterry W, Tilgen W, Andre S, et al. CD4+CD7− leukemic T cells from patients with Sezary syndrome are protected from galectin-1-triggered T cell death. Leukemia. 2002 May; 16(5):840-5.
12. Png Y T, Vinanica N, Kamiya T, Shimasaki N, Coustan-Smith E, Campana D. Blockade of CD7 expression in T cells for effective chimeric antigen receptor targeting of T-cell malignancies. Blood Adv. 2017 Nov. 28; 1(25):2348-60.
13. Kita K, Miwa H, Nakase K, Kawakami K, Kobayashi T, Shirakawa S, et al. Clinical importance of CD7 expression in acute myelocytic leukemia. The Japan Cooperative Group of Leukemia/Lymphoma. Blood. 1993 May 1; 81(9):2399-405.
14. Rohrs S, Scherr M, Romani J, Zaborski M, Drexler H G, Quentmeier H. CD7 in acute myeloid leukemia: correlation with loss of wild-type CEBPA, consequence of epigenetic regulation. J Hematol OncolJ Hematol Oncol. 2010 Apr. 14; 3:15.
15. Keizer M P, Kamp A M, Aarts C, Geisler J, Caron H N, van de Wetering M D, et al. The High Prevalence of Functional Complement Defects Induced by Chemotherapy. Front Immunol. 2016; 7:420.
16. Diebolder C A, Beurskens F J, de Jong R N, Koning R I, Strumane K, Lindorfer M A, et al.

Complement is activated by IgG hexamers assembled at the cell surface. Science. 2014 Mar. 14; 343(6176):1260-3.
17. Heinrich G, Gram H, Kocher H P, Schreier M H, Ryffel B, Akbar A, et al. Characterization of a human T cell-specific chimeric antibody (CD7) with human constant and mouse variable regions. J Immunol Baltim Md 1950. 1989 Dec. 1; 143(11):3589-97.
18. Ravandi F, Aribi A, O'Brien S, Faderl S, Jones D, Ferrajoli A, et al. Phase II study of alemtuzumab in combination with pentostatin in patients with. J Clin Oncol Off J Am Soc Clin Oncol. 2009 Nov. 10; 27(32):5425-30.
19. Cooper M L, Choi J, Staser K, Ritchey J K, Devenport J M, Eckardt K, et al. An “off-the-shelf” fratricide-resistant CAR-T for the treatment of T cell hematologic malignancies. Leukemia. 2018 Feb. 20;
20. Haapaniemi E, Botla S, Persson J, Schmierer B, Taipale J. CRISPR-Cas9 genome editing induces a p53-mediated DNA damage response. Nat Med. 2018 July; 24(7):927-30.
21. Kosicki M, Tomberg K, Bradley A. Repair of double-strand breaks induced by CRISPR-Cas9 leads to large deletions and complex rearrangements. Nat Biotechnol. 2018 September; 36(8):765-71.
22. Van Vlierberghe P, Ferrando A. The molecular basis of T cell acute lymphoblastic leukemia. J Clin Invest. 2012 October; 122(10):3398-406.
23. Coustan-Smith E, Mullighan C G, Onciu M, Behm F G, Raimondi S C, Pei D, et al. Early T-cell precursor leukaemia: a subtype of very high-risk acute lymphoblastic leukaemia. Lancet Oncol. 2009 February; 10(2):147-56.
24. Marks D I, Paietta E M, Moorman A V, Richards S M, Buck G, DeWald G, et al. T-cell acute lymphoblastic leukemia in adults: clinical features, immunophenotype, cytogenetics, and outcome from the large randomized prospective trial (UKALL XII/ECOG 2993). Blood. 2009 Dec. 10; 114(25):5136-45.
25. Aldoss I, Bargou R C, Nagorsen D, Friberg G R, Baeuerle P A, Forman S J. Redirecting T cells to eradicate B-cell acute lymphoblastic leukemia: bispecific. Leukemia. 2017 April; 31(4):777-87.
26. Lee E-C, Liang Q, Ali H, Bayliss L, Beasley A, Bloomfield-Gerdes T, et al. Complete humanization of the mouse immunoglobulin loci enables efficient therapeutic antibody discovery. Nat Biotechnol. 2014 April; 32(4):356-63.
27. Phanse Y, Ramer-Tait A E, Friend S L, Carrillo-Conde B, Lueth P, Oster C J, et al. Analyzing cellular internalization of nanoparticles and bacteria by multi-spectral imaging flow cytometry. United States; 2012.
28. de Jong R N, Beurskens F J, Verploegen S, Strumane K, van Kampen M D, Voorhorst M, et al. A Novel Platform for the Potentiation of Therapeutic Antibodies Based on Antigen-Dependent Formation of IgG Hexamers at the Cell Surface. PLoS Biol. 2016 January; 14(1):e1002344.
29. Agnusdei V, Minuzzo S, Frasson C, Grassi A, Axelrod F, Satyal S, et al. Therapeutic antibody targeting of Notch1 in T-acute lymphoblastic leukemia xenografts. Leukemia. 2014 February; 28(2):278-88.
30. Jung L K, Roy A K, Chakkalath H R. CD7 augments T cell proliferation via the interleukin-2 autocrine pathway. Cell Immunol. 1992 Apr. 15; 141(1):189-99.
31. Markiewski M M, Lambris J D. The role of complement in inflammatory diseases from behind the scenes into the spotlight. Am J Pathol. 2007 September; 171(3):715-27.
32. Kola A, Baensch M, Bautsch W, Hennecke M, Klos A, Casaretto M, et al. Epitope mapping of a C5a neutralizing mAb using a combined approach of phage display, synthetic peptides and site-directed mutagenesis. Immunotechnology Int J Immunol Eng. 1996 June; 2(2):115-26.
33. Baum W, Steininger H, Bair H J, Becker W, Hansen-Hagge T E, Kressel M, et al. Therapy with CD7 monoclonal antibody TH-69 is highly effective for xenografted human T-cell ALL. Br J Haematol. 1996 November; 95(2):327-38.
34. Matlung H L, Babes L, Zhao X W, van Houdt M, Treffers L W, van Rees D J, et al. Neutrophils Kill Antibody-Opsonized Cancer Cells by Trogoptosis. Cell Rep. 2018 Jun. 26; 23(13):3946-3959.e6.
35. Frankel A E, Laver J H, Willingham M C, Burns U, Kersey J H, Vallera D A. Therapy of patients with T-cell lymphomas and leukemias using an anti-CD7 monoclonal antibody-ricin A chain immunotoxin. Leuk Lymphoma. 1997 July; 26(3-4):287-98.
36. DeAngelo D J, Yu D, Johnson J L, Coutre S E, Stone R M, Stopeck A T, et al. Nelarabine induces complete remissions in adults with relapsed or refractory. Blood. 2007 Jun. 15; 109(12):5136-42.
37. Jain N, Lamb A V, O'Brien S, Ravandi F, Konopleva M, Jabbour E, et al. Early T-cell precursor acute lymphoblastic leukemia/lymphoma (ETP-ALL/LBL) in adolescents and adults: a high-risk subtype. Blood. 2016 Apr. 14; 127(15):1863-9.

### Glossary

AML acute myeloid leukaemia
ADCC antibody-dependent cell-mediated cytotoxicity
ADCP antibody-dependent cell-mediated phagocytosis
CAR-T chimeric antigen receptor T cells
CDC complement-dependent cytotoxicity
CEBPA CCAAT/enhancer-binding protein alpha gene
CRP complement regulatory protein
DC Developmental Candidate
HSC haematopoietic stem cell
mAb monoclonal antibody
MOA mode of action
MRD minimal residual disease
PBMC peripheral blood mononuclear cell
SoC standard of care
Tagg aggregation temperature
T-ALL T-cell acute lymphoblastic leukaemia
Tm melting temperature
T-PLL T-cell prolymphocytic leukemia. Glossary
AML acute myeloid leukaemia
ADCC antibody-dependent cell-mediated cytotoxicity
ADCP antibody-dependent cell-mediated phagocytosis
CAR-T chimeric antigen receptor T cells
CDC complement-dependent cytotoxicity
CEBPA CCAAT/enhancer-binding protein alpha gene
CRP complement regulatory protein
DC Developmental Candidate
HSC haematopoietic stem cell
mAb monoclonal antibody
MOA mode of action
MRD minimal residual disease
PBMC peripheral blood mononuclear cell
SoC standard of care
Tagg aggregation temperature
T-ALL T-cell acute lymphoblastic leukaemia
Tm melting temperature
T-PLL T-cell prolymphocytic leukemia

TABLE 1
Affinity characterization measured by SPR. Kinetic rates and Equilibrium
binding constants from SPR of mAbs were obtained by global fitting
of sensorgrams using a 1:1 Interaction Model.

| | Human CD7 | | | Cynomolgus CD7 | | |
|---|---|---|---|---|---|---|
| | ka, M⁻¹s⁻¹ | kd, s⁻¹ | KD, nM | ka, M⁻¹s⁻¹ | kd, s⁻¹ | KD, nM |
| RFT2 | 6.11 × 10⁵ | 3.70 × 10⁻³ | 6.225 | 5.29 × 10⁵ | 2.54 × 10⁻² | 50.765 |
| TH69 | 4.51 × 10⁵ | 1.25 × 10⁻⁴ | 0.279 | nbs | nbs | nbs |
| 1741G09 | 4.16 × 10⁵ | 1.92 × 10⁻³ | 4.735 | 4.24 × 10⁵ | 6.11 × 10⁻⁴ | 0.145 |
| 1734F05 | 3.69 × 10⁵ | 3.56 × 10⁻⁴ | 0.980 | 1.65 × 10⁵ | 6.42 × 10⁻⁴ | 3.895 |

nbs = No binding observed.

TABLE 2
Maximum killing and EC50 values for lead mAbs on CEM
cells in the CDC assays. The mean max killing and EC50
values were obtained from the experiments in FIG. 5.

| MAbs | Maximum killing | EC₅₀ |
|---|---|---|
| 1730C02 E345R | 93.2% | 0.37 pM |
| 1734E10 E345R | 8.8% | 0.09 nM |
| 1734F05 E345R | 100.1% | 1.16 nM |
| 1738B07 E345R | 75.4% | 2.25 nM |
| 1741E04 E345R | 99.8% | 0.12 nM |
| 1741G09 E345R | 99.5% | 0.12 nM |
| 1896A03 E345R | 69.5% | 0.60 nM |
| TH69 E345R | 87.1% | 0.21 nM |
| RFT2 E345R | 98.5% | 0.22 nM |

TABLE 4
Cytokine levels from PBMCs treated with 1741G09 E430G and control antibodies. IgG1 and IgG1 E430G
antibodies were included as negative controls, and anti-CD3 and anti-CD28 as positive controls. PBMCs
from 5 donors were treated with different antibodies at the concentration of 60 μg/ml at 37° C. for 48 hours.
1741G09 E430G

| Cytokines | mAb, 60 | Donor 1 | Donor 2 | Donor 3 | Donor 4 | Donor 5 | Mean | SD | p value | p value |
|---|---|---|---|---|---|---|---|---|---|---|
| IFNg | 1731G09 | 379.4 | 33.5 | 44.2 | 119.6 | 37.3 | 122.8 | 379.4 | 0.0688 | 0.0185 |
| | IgG1 | 324.0 | 16.4 | 21.1 | 48.3 | 25.1 | 87.0 | 324.0 | 0.1957 | |
| | IgG1 | 205.5 | 17.0 | 26.2 | 37.5 | 33.2 | 63.9 | 205.5 | | |
| | anti-CD3 | 81768.0 | 15838.5 | 104409.0 | 79869.9 | 16352.7 | 59647.6 | 104409.0 | 0.0156 | |
| | anti-CD28 | 176706.2 | 39510.9 | 332610.1 | 234603.9 | 27971.4 | 162280.5 | 332610.1 | 0.0246 | |
| IL-2 | 1731G09 | 27.2 | 6.3 | — | 39.4 | 48.8 | 30.4 | 48.8 | 0.1885 | 0.1410 |
| | IgG1 | 27.1 | 12.1 | 3.8 | 17.7 | 27.7 | 17.7 | 27.7 | 0.4268 | |
| | IgG1 | 25.4 | 13.1 | 1.5 | 32.1 | 11.5 | 16.7 | 32.1 | | |
| | anti-CD3 | 21.1 | 30.5 | 72.4 | 173.0 | 14.7 | 62.3 | 173.0 | 0.0845 | |
| | anti-CD28 | 19123.8 | 7147.5 | 29754.7 | 23012.6 | 4167.3 | 16641.2 | 29754.7 | 0.0130 | |
| IL-8 | 1731G09 | 47373.5 | 39571.9 | 18384.7 | 55423.0 | 25245.7 | 37199.8 | 55423.0 | 0.0035 | 0.0157 |
| | IgG1 | 41038.1 | 30897.0 | 18371.4 | 40096.4 | 12786.3 | 28637.8 | 41038.1 | 0.0037 | |
| | IgG1 | 17713.0 | 18059.4 | 9150.8 | 17990.1 | 3425.5 | 13267.8 | 18059.4 | | |
| | anti-CD3 | 23594.2 | 40748.8 | 23779.7 | 26202.6 | 38104.2 | 30485.9 | 40748.8 | 0.0153 | |
| | anti-CD28 | 23022.4 | 61283.1 | 38889.5 | 58715.7 | 41168.1 | 44615.8 | 61283.1 | 10.0052 | |
| Mip-1α | 1731G09 | 3818.5 | 1850.7 | 705.4 | 3436.7 | 360.7 | 2034.4 | 3818.5 | 0.0240 | 0.0676 |
| | IgG1 | 3240.0 | 1991.0 | 593.9 | 2650.1 | 147.5 | 1724.5 | 3240.0 | 0.0162 | |
| | IgG1 | 1188.1 | 662.7 | 194.1 | 722.4 | — | 691.8 | 1188.1 | | |
| | anti-CD3 | 28382.7 | 14726.4 | 18475.3 | 29620.4 | 23177.8 | 22876.5 | 29620.4 | 0.0042 | |
| | anti-CD28 | 32584.0 | 9903.4 | 23311.9 | 28880.2 | 13288.8 | 21593.7 | 32584.0 | 0.0091 | |
| TNFα | 1731G09 | 1283.5 | 486.3 | 662.4 | 1427.8 | 1157.8 | 1003.6 | 1427.8 | 0.0047 | 0.0333 |
| | IgG1 | 1190.3 | 319.9 | 590.8 | 936.5 | 493.1 | 706.1 | 1190.3 | 0.0044 | |
| | IgG1 | 496.4 | 137.5 | 211.0 | 247.1 | 92.0 | 236.8 | 496.4 | | |
| | anti-CD3 | 12810.6 | 10502.9 | 10746.7 | 15180.0 | 10199.3 | 11887.9 | 15180.0 | 0.0001 | |
| | anti-CD28 | 34118.4 | 14593.3 | 46209.1 | 28840.4 | 13741.9 | 27500.6 | 46209.1 | 0.0055 | |
| IL-10 | 1731G09 | 31.4 | 20.1 | 35.9 | 33.7 | 83.1 | 40.8 | 83.1 | 0.1318 | 0.0778 |
| | IgG1 | 28.1 | 11.7 | 37.3 | 22.4 | 45.7 | 29.0 | 45.7 | 0.3309 | |
| | IgG1 | 34.3 | 9.5 | 38.2 | 25.0 | 28.7 | 27.1 | 38.2 | | |
| | anti-CD3 | 13934.0 | 5430.3 | 93210.8 | 20.2 | 8704.4 | 24259.9 | 93210.8 | 0.1179 | |
| | anti-CD28 | 5379.4 | 1391.5 | 6882.0 | 23.6 | 1192.3 | 2973.7 | 6882.0 | 0.0453 | |
| IL-12p40 | 1731G09 | 10.1 | — | — | — | — | 10.1 | 10.1 | | |
| | IgG1 | 10.0 | — | — | — | — | 10.0 | 10.0 | | |
| | IgG1 | 12.8 | — | — | — | — | 12.8 | 12.8 | | |
| | anti-CD3 | 29.1 | — | 43.0 | — | 14.7 | 29.0 | 43.0 | | |
| | anti-CD28 | 9.5 | — | 21.7 | — | — | 15.6 | 21.7 | | |
| IL-17A | 1731G09 | 7.0 | 1.0 | 18.1 | 4.9 | 16.8 | 9.6 | 18.1 | 0.1079 | 0.1130 |
| | IgG1 | 8.3 | 0.9 | 14.9 | 2.3 | 4.9 | 6.3 | 14.9 | 0.2687 | |
| | IgG1 | 6.8 | 1.3 | 13.6 | 4.0 | 3.5 | 5.8 | 13.6 | | |
| | anti-CD3 | 2812.6 | 1008.4 | 2468.3 | 6.1 | 2723.2 | 1803.7 | 2812.6 | 0.0158 | |
| | anti-CD28 | 2801.1 | 1136.5 | 2424.8 | 5.6 | 1464.9 | 1566.6 | 2801.1 | 0.0170 | |
| IL-1β | 1731G09 | 66.0 | 55.7 | 9.7 | 64.2 | 37.5 | 46.6 | 66.0 | 0.0043 | 0.0130 |
| | IgG1 | 26.0 | 15.0 | 10.4 | 43.1 | 6.4 | 20.2 | 43.1 | 0.0193 | |
| | IgG1 | 15.9 | 11.0 | 2.4 | 20.7 | 1.1 | 10.2 | 20.7 | | |
| | anti-CD3 | 728.9 | 419.1 | 740.0 | 14.5 | 315.6 | 443.6 | 740.0 | 0.0173 | |
| | anti-CD28 | 687.5 | 341.3 | 454.0 | 15.1 | 198.0 | 339.2 | 687.5 | 0.0226 | |
| IL-6 | 1731G09 | 188.6 | 208.3 | 106.2 | 219.6 | 32.1 | 151.0 | 219.6 | 0.0043 | 0.0579 |
| | IgG1 | 159.4 | 116.7 | 105.0 | 205.9 | 10.3 | 119.5 | 205.9 | 0.0154 | |
| | IgG1 | 115.8 | 105.6 | 60.5 | 157.8 | 5.7 | 89.1 | 157.8 | | |
| | anti-CD3 | 2055.5 | 506.1 | 2890.9 | 114.3 | 533.4 | 1220.0 | 2890.9 | 0.0520 | |
| | anti-CD28 | 2408.0 | 570.3 | 2475.5 | 113.7 | 384.0 | 1190.3 | 2475.5 | 0.0505 | |

TABLE 5
Immuno-phenotype categorisation of
T-ALL/LBL using WHO criteria (37)

| | CD1a | CD2 | cCD3 | sCD3 | CD4 | CD8 | CD7 | CD34 |
|---|---|---|---|---|---|---|---|---|
| Pro-T | − | − | + | − | − | − | + | +/− |
| Pre-T | − | + | + | − | − | − | + | +/− |
| Cortical T | + | + | + | − | + | + | + | − |
| (Thymic T) | | | | | | | | |

| | | | | | | | |
|---|---|---|---|---|---|---|---|
| Medullary T | − | + | + | + | Either CD4+ | + | − |
| (Mature T) | | | | | or CD8+ | | |
| | | | | | (but not both) | | |

TABLE 6
The statistical comparison of the antibodies is presented in FIG. 6

| | Mean | | | Adjusted |
|---|---|---|---|---|
| Holm-Sidak's multiple comparisons test | Diff. | Significant | Summary | P Value |
| 1730C02 E345R vs. 1734E10 E345R | 95.95 | Yes | **** | <0.0001 |
| 1730C02 E345R vs. 1734F05 E345R | 50.83 | Yes | ** | 0.0062 |
| 1730C02 E345R vs. 1738B07 E345R | 60.17 | Yes | ** | 0.0012 |
| 1730C02 E345R vs. 1741E04 E345R | −40.73 | Yes | ** | 0.0368 |
| 1730C02 E345R vs. 1741G09 E345R | −38.27 | Yes | * | 0.0491 |
| 1730C02 E345R vs. 1896A03 E345R | 40.24 | Yes | * | 0.038 |
| 1730C02 E345R vs. TH69 E345R | −2.167 | No | ns | 0.9728 |
| 1730C02 E345R vs. RFT2 E345R | −14.1 | No | ns | 0.8255 |
| 1730C02 E345R vs. Isotype control E345R | 110.7 | Yes | **** | <0.0001 |
| 1734E10 E345R vs. 1734F05 E345R | −45.11 | Yes | * | 0.0175 |
| 1734E10 E345R vs. 1738B07 E345R | −35.77 | No | ns | 0.0675 |
| 1734E10 E345R vs. 1741E04 E345R | −136.7 | Yes | **** | <0.0001 |
| 1734E10 E345R vs. 1741G09 E345R | −134.2 | Yes | **** | <0.0001 |
| 1734E10 E345R vs. 1896A03 E345R | 55.71 | Yes | ** | 0.0027 |
| 1734E10 E345R vs. TH69 E345R | −98.11 | Yes | **** | <0.0001 |
| 1734E10 E345R vs. RFT2 E345R | −110 | Yes | **** | <0.0001 |
| 1734E10 E345R vs. Isotype control E345R | 14.78 | No | ns | 0.8255 |
| 1734F05 E345R vs. 1738B07 E345R | 9.34 | No | ns | 0.8542 |
| 1734F05 E345R vs. 1741E04 E345R | −91.57 | Yes | **** | <0.0001 |
| 1734F05 E345R vs. 1741G09 E345R | −89.1 | Yes | **** | <0.0001 |
| 1734F05 E345R vs. 1896A03 E345R | −10.59 | No | ns | 0.8542 |
| 1734F05 E345R vs. TH69 E345R | −53 | Yes | ** | 0.0042 |
| 1734F05 E345R vs. RFT2 E345R | −64.93 | Yes | *** | 0.0005 |
| 1734F05 E345R vs. Isotype control E345R | 59.89 | Yes | ** | 0.0012 |
| 1738B07 E345R vs. 1741E04 E345R | −100.9 | Yes | **** | <0.0001 |
| 1738B07 E345R vs. 1741G09 E345R | −98.44 | Yes | **** | <0.0001 |
| 1738B07 E345R vs. 1896A03 E345R | −19.93 | No | ns | 0.5836 |
| 1738B07 E345R vs. TH69 E345R | −62.34 | Yes | *** | 0.0008 |
| 1738B07 E345R vs. RFT2 E345R | −74.27 | Yes | **** | <0.0001 |
| 1738B07 E345R vs. Isotype control E345R | 50.55 | Yes | ** | 0.0063 |
| 1741E04 E345R vs. 1741G09 E345R | 2.467 | No | ns | 0.9728 |
| 1741E04 E345R vs. 1896A03 E345R | 80.97 | Yes | **** | <0.0001 |
| 1741E04 E345R vs. TH69 E345R | 38.57 | Yes | * | 0.0491 |
| 1741E04 E345R vs. RFT2 E345R | 26.63 | No | ns | 0.2915 |
| 1741E04 E345R vs. Isotype control E345R | 151.5 | Yes | **** | <0.0001 |
| 1741G09 E345R vs. 1896A03 E345R | 78.51 | Yes | **** | <0.0001 |
| 1741G09 E345R vs. TH69 E345R | 36.1 | No | ns | 0.0675 |
| 1741G09 E345R vs. RFT2 E345R | 24.17 | No | ns | 0.3812 |
| 1741G09 E345R vs. Isotype control E345R | 149 | Yes | **** | <0.0001 |
| 1896A03 E345R vs. TH69 E345R | −42.41 | Yes | * | 0.0282 |
| 1896A03 E345R vs. RFT2 E345R | −54.34 | Yes | ** | 0.0034 |
| 1896A03 E345R vs. Isotype control E345R | 70.49 | Yes | *** | 0.0002 |
| TH69 E345R vs. RFT2 E345R | −11.93 | No | ns | 0.8542 |
| TH69 E345R vs. Isotype control E345R | 112.9 | Yes | **** | <0.0001 |
| RFT2 E345R vs. Isotype control E345R | 124.8 | Yes | **** | <0.0001 |

TABLE 8
Biophysical summary for 1741G09 E430G

| Research code | 1741G09 E430G |
|---|---|
| Immunogen | Recombinant human and cynomolgus CD7 MEFs |
| | (mouse embryonic fibroblasts) |
| Affinity to soluble | Human CD7: 4.75 nM; cynomolgus CD7: |
| CD7 | 0.15 nM |
| EC₅₀ value for CEM | 100-150 pM; maximum killing ~100% |
| cell | |
| depletion in the in | |
| vitro human CDC | |
| assay | |
| Transient expression/ | 345 to 679 mg/L |
| CHOE7 (pTT5) | |
| Stable expression | 353 to 445 mg/L |
| pools/Lonza Xceed | |
| CHO | |
| Tm | 73.0° C. in 10 mM sodium succinate, 50 mM |
| | Arginine-HCl, pH5.5 |
| Impurities | No issue with aggregates, purity >95% of Protein |
| | A |
| Biophysical data | No significant change in aggregation, |
| | fragmentation and activity after freeze thaw, |
| | force deamidation and acidic hold. Following |
| | oxidation stress, decrease in activity is H202 |
| | concentration dependant |

TABLE 9
Sequences
Sequences for use in the present invention.

| Seq | | | | |
|---|---|---|---|---|
| ID | | | | |
| No: | | | Description | Sequence |
| 1 | G09 | CDRH1 | Amino acid sequence | GFSFSVAW |
| | | (IMGT) | of CDRH1 using IMGT | |
| 2 | G09 | CDRH2 | Amino acid sequence | IKRKTDGGTT |
| | | (IMGT) | of CDRH2 using IMGT | |
| 3 | G09 | CDRH3 | Amino acid sequence | TTDPLLRYSDWLSHSLMGDV |
| | | (IMGT) | of CDRH3 using IMGT | |
| 4 | G09 | CDRH1 | Amino acid sequence | SVAWMS |
| | | (KABAT) | of CDRH1 using Kabat | |
| 5 | G09 | CDRH2 | Amino acid sequence | RIKRKTDGGTTDYAAPVKG |
| | | (KABAT) | of CDRH2 using Kabat | |
| 6 | G09 | CDRH3 | Amino acid sequence | DPLLRYSDWLSHSLMGDV |
| | | (KABAT) | of CDRH3 using Kabat | |
| 7 | G09 | Heavy | Amino acid sequence | EVQLVESGGGLVKPGGSLRLSCAASGFSFSVAWMSWVRQ |
| | | chain | of VH | APGKGLEWVGRIKRKTDGGTTDYAAPVKGRFIISRDDSIKA |
| | | variable | IGHV3-15*01; | LYLQMNSLKTEDTAVYYCTTDPLLRYSDWLSHSLMGDVW |
| | | region | IGHD3-9*01; | GQGTTVTVSS |
| | | | IGHJ6*02 | |
| | | | CDRH3 = 20 a.a.s | |
| | | | Non-germline | |
| | | | residues = 17 | |
| | | | a.a.s (including | |
| | | | junctional | |
| | | | insertion and | |
| | | | hypermutation) | |
| 8 | G09 | Heavy | Nucleotide sequence | GAGGTGCAGCTGGTGGAGTCTGGGGGAGGCTTGGTAA |
| | | chain | of V_{H} | AGCCTGGGGGGTCCCTTAGACTCTCCTGTGCAGCCTCTG |
| | | variable | | GATTCAGTTTCAGTGTCGCCTGGATGAGCTGGGTCCGCC |
| | | region | | AGGCTCCAGGGAAGGGGCTGGAGTGGGTTGGCCGTATT |
| | | | | AAGCGCAAAACTGATGGTGGGACAACAGACTACGCTGC |
| | | | | ACCCGTGAAAGGCAGATTCATCATCTCAAGAGATGATTC |
| | | | | AATAAAAGCGCTGTATCTGCAAATGAACAGCCTGAAAA |
| | | | | CCGAGGACACAGCCGTGTATTACTGTACCACAGATCCCT |
| | | | | TATTACGATATTCTGACTGGTTATCCCACTCCCTTATGGG |
| | | | | GGACGTCTGGGGCCAAGGGACCACGGTCACCGTCTCCT |
| | | | | CAG |
| 9 | G09 | Full | Amino acid sequence | EVQLVESGGGLVKPGGSLRLSCAASGFSFSVAWMSWVRQ |
| | | heavy | heavy chain | APGKGLEWVGRIKRKTDGGTTDYAAPVKGRFIISRDDSIKA |
| | | chain | | LYLQMNSLKTEDTAVYYCTTDPLLRYSDWLSHSLMGDVW |
| | | sequence | | GQGTTVTVSSASTKGPSVFPLAPSSKSTSGGTAALGCLVKD |
| | | | | YFPEPVTVSWNSGALTSGVHTFPAVLQSSGLYSLSSVVTVP |
| | | | | SSSLGTQTYICNVNHKPSNTKVDKKVEPKSCDKTHTCPPCP |
| | | | | APELLGGPSVFLFPPKPKDTLMISRTPEVTCVVVDVSHEDP |
| | | | | EVKFNWYVDGVEVHNAKTKPREEQYNSTYRVVSVLTVLH |
| | | | | QDWLNGKEYKCKVSNKALPAPIEKTISKAKGQPREPQVYTL |
| | | | | PPSRDELTKNQVSLTCLVKGFYPSDIAVEWESNGQPENNY |
| | | | | KTTPPVLDSDGSFFLYSKLTVDKSRWQQGNVFSCSVMHG |
| | | | | ALHNHYTQKSLSLSPG |
| 10 | G09 | Full | Nucleic acid sequence | GAGGTGCAGCTGGTGGAGTCTGGGGGAGGCTTGGTAA |
| | | heavy | heavy chain | AGCCTGGGGGGTCCCTTAGACTCTCCTGTGCAGCCTCTG |
| | | chain | | GATTCAGTTTCAGTGTCGCCTGGATGAGCTGGGTCCGCC |
| | | sequence | | AGGCTCCAGGGAAGGGGCTGGAGTGGGTTGGCCGTATT |
| | | | | AAGCGCAAAACTGATGGTGGGACAACAGACTACGCTGC |
| | | | | ACCCGTGAAAGGCAGATTCATCATCTCAAGAGATGATTC |
| | | | | AATAAAAGCGCTGTATCTGCAAATGAACAGCCTGAAAA |
| | | | | CCGAGGACACAGCCGTGTATTACTGTACCACAGATCCCT |
| | | | | TATTACGATATTCTGACTGGTTATCCCACTCCCTTATGGG |
| | | | | GGACGTCTGGGGCCAAGGGACCACGGTCACCGTCTCCT |
| | | | | CAGGCCAGCACCAAGGGCCCCTCTGTGTTCCCTCTGGCC |
| | | | | CCTTCCAGCAAGTCCACCTCTGGCGGAACAGCCGCTCTG |
| | | | | GGCTGCCTCGTGAAGGACTACTTCCCCGAGCCTGTGACC |
| | | | | GTGTCCTGGAACTCTGGCGCTCTGACCAGCGGAGTGCA |
| | | | | CACCTTCCCTGCTGTGCTGCAGTCCTCCGGCCTGTACTCC |
| | | | | CTGTCCTCCGTCGTGACCGTGCCTTCCAGCTCTCTGGGC |
| | | | | ACCCAGACCTACATCTGCAACGTGAACCACAAGCCCTCC |
| | | | | AACACCAAGGTGGACAAGAAGGTGGAACCCAAGTCCTG |
| | | | | CGACAAGACCCACACCTGTCCCCCTTGTCCTGCCCCTGA |
| | | | | ACTGCTGGGCGGACCTTCCGTGTTCCTGTTCCCCCCAAA |
| | | | | GCCCAAGGACACCCTGATGATCTCCCGGACCCCCGAAGT |
| | | | | GACCTGCGTGGTGGTGGATGTGTCCCACGAGGACCCTG |
| | | | | AAGTGAAGTTCAATTGGTACGTGGACGGCGTGGAAGTG |
| | | | | CACAACGCCAAGACCAAGCCTAGAGAGGAACAGTACAA |
| | | | | CTCCACCTACCGGGTGGTGTCCGTGCTGACCGTGCTGCA |
| | | | | CCAGGATTGGCTGAACGGCAAAGAGTACAAGTGCAAGG |
| | | | | TGTCCAACAAGGCCCTGCCTGCCCCCATCGAAAAGACCA |
| | | | | TCTCCAAGGCCAAGGGCCAGCCCCGGGAACCCCAGGTG |
| | | | | TACACACTGCCCCCTAGCAGGGACGAGCTGACCAAGAA |
| | | | | CCAGGTGTCCCTGACCTGTCTCGTGAAAGGCTTCTACCC |
| | | | | CTCCGATATCGCCGTGGAATGGGAGTCCAACGGCCAGC |
| | | | | CTGAGAACAACTACAAGACCACCCCCCCTGTGCTGGACT |
| | | | | CCGACGGCTCATTCTTCCTGTACAGCAAGCTGACAGTGG |
| | | | | ACAAGTCCCGGTGGCAGCAGGGCAACGTGTTCTCCTGCT |
| | | | | CCGTGATGCACGGCGCCCTGCACAACCACTACACCCAGA |
| | | | | AGTCCCTGTCCCTGAGCCCCGGC |
| 11 | G09 | CDRL1 | Amino acid sequence | QNINNY |
| | | (IMGT) | of CDRL1 using IMGT | |
| 12 | G09 | CDRL2 | Amino acid sequence | AAS |
| | | (IMGT) | of CDRL2 using IMGT | |
| 13 | G09 | CDRL3 | Amino acid sequence | QQTYSIPLTFGGG |
| | | (IMGT) | of CDRL3 using IMGT | |
| 14 | G09 | CDRL1 | Amino acid sequence | NNYLN |
| | | (KABAT) | of CDRL1 using KABAT | |
| 15 | G09 | CDRL2 | Amino acid sequence | YAASSLQSGVPS |
| | | (KABAT) | of CDRL2 using KABAT | |
| 16 | G09 | CDRL3 | Amino acid sequence | QQTYSIPLTFGGG |
| | | (KABAT) | of CDRL3 using KABAT | |
| 17 | G09 | Light | Amino acid sequence | DIQMTQSPSSLSASVGDRVAITCRTSQNINNYLNWYQQKP |
| | | chain | of VL | GKAPKPLIYAASSLQSGVPSRFSGSGSGTDFTLTISTLHPEDF |
| | | variable | IGKV1D-39*01; | ATYYCQQTYSIPLTFGGGTKVEIK |
| | | region | IGKJ4*01 | |
| | | | CDRK3 = 9 a.a.s | |
| | | | Non-germline | |
| | | | residues = 10 | |
| | | | a. a. s | |
| | | | F-Serine | |
| | | | present at this | |
| | | | position in the | |
| | | | sequence | |
| | | | obgtained from | |
| | | | the hybridoma ; | |
| | | | Serine was back | |
| | | | engineered to | |
| | | | Phenylalanine to | |
| | | | improve the | |
| | | | expression | |
| 18 | G09 | Light | Nucleic acid sequence | GATATCCAGATGACCCAGTCCCCCTCCAGCCTGTCTGCCT |
| | | chain | of V_{L} | CTGTGGGCGACAGAGTGGCCATCACCTGTCGGACCTCCC |
| | | variable | | AGAACATCAACAACTACCTGAACTGGTATCAGCAGAAG |
| | | region | | CCCGGCAAGGCCCCCAAGCCCCTGATCTACGCTGCTTCC |
| | | | | AGTCTGCAGTCCGGCGTGCCCTCTAGATTCTCCGGCTCT |
| | | | | GGCTCTGGCACCGACTTCACCCTGACCATCTCCACCCTGC |
| | | | | ACCCCGAGGACTTCGCCACCTACTACTGCCAGCAGACCT |
| | | | | ACTCCATCCCCCTGACCTTTGGCGGAGGCACCAAGGTGG |
| | | | | AAATCAAA |
| 19 | G09 | Full | Amino acid sequence | DIQMTQSPSSLSASVGDRVAITCRTSQNINNYLNWYQQKP |
| | | light | light chain | GKAPKPLIYAASSLQSGVPSRFSGSGSGTDFTLTISTLHPEDF |
| | | chain | | ATYYCQQTYSIPLTFGGGTKVEIKRTVAAPSVFIFPPSDEQLK |
| | | sequence | | SGTASVVCLLNNFYPREAKVQWKVDNALQSGNSQESVTE |
| | | | | QDSKDSTYSLSSTLTLSKADYEKHKVYACEVTHQGLSSPVTK |
| | | | | SFNRGEC |
| 20 | G09 | Full | Nucleic acid sequence | GATATCCAGATGACCCAGTCCCCCTCCAGCCTGTCTGCCT |
| | | light | light chain | CTGTGGGCGACAGAGTGGCCATCACCTGTCGGACCTCCC |
| | | chain | | AGAACATCAACAACTACCTGAACTGGTATCAGCAGAAG |
| | | sequence | | CCCGGCAAGGCCCCCAAGCCCCTGATCTACGCTGCTTCC |
| | | | | AGTCTGCAGTCCGGCGTGCCCTCTAGATTCTCCGGCTCT |
| | | | | GGCTCTGGCACCGACTTCACCCTGACCATCTCCACCCTGC |
| | | | | ACCCCGAGGACTTCGCCACCTACTACTGCCAGCAGACCT |
| | | | | ACTCCATCCCCCTGACCTTTGGCGGAGGCACCAAGGTGG |
| | | | | AAATCAAACGTACGGTGGCCGCTCCCTCCGTGTTCATCT |
| | | | | TCCCACCTTCCGACGAGCAGCTGAAGTCCGGCACCGCTT |
| | | | | CTGTCGTGTGCCTGCTGAACAACTTCTACCCCCGCGAGG |
| | | | | CCAAGGTGCAGTGGAAGGTGGACAACGCCCTGCAGTCC |
| | | | | GGCAACTCCCAGGAATCCGTGACCGAGCAGGACTCCAA |
| | | | | GGACAGCACCTACTCCCTGTCCTCCACCCTGACCCTGTCC |
| | | | | AAGGCCGACTACGAGAAGCACAAGGTGTACGCCTGCGA |
| | | | | AGTGACCCACCAGGGCCTGTCTAGCCCCGTGACCAAGTC |
| | | | | TTTCAACCGGGGCGAGTGT |
| 21 | F05 | CDRH1 | Amino acid sequence | QSVSSY |
| | | (IMGT) | of CDRH1 using IMGT | |
| 22 | F05 | CDRH2 | Amino acid sequence | DTS |
| | | (IMGT) | of CDRH2 using IMGT | |
| 23 | F05 | CDRH3 | Amino acid sequence | QQRRNWPLT |
| | | (IMGT) | of CDRH3 using IMGT | |
| 24 | F05 | CDRH1 | Amino acid sequence | RVSQSVSSYLA |
| | | (KABAT) | of CDRH1 using Kabat | |
| 25 | F05 | CDRH2 | Amino acid sequence | DTSNRAT |
| | | (KABAT) | of CDRH2 using Kabat | |
| 26 | F05 | CDRH3 | Amino acid sequence | QQRRNWPLT |
| | | (KABAT) | of CDRH3 using Kabat | |
| 27 | F05 | Heavy | Amino acid sequence | EVQLVESGGGLVQPGGSLRLSCAASGFTFSRYAMSWVRQ |
| | | chain | of V_{H} | APGKGLEWVSSISASGATTFYADPVKGRFTISRDNSKKTLYL |
| | | variable | | QMNSLRAEDTAVYYCAKDQDFDILTGYLNWFDPWGQGT |
| | | region | | LVTVSS |
| 28 | F05 | Heavy | Nucleotide sequence | GAAGTGCAGCTGGTTGAATCTGGCGGCGGATTGGTTCA |
| | | chain | of V_{H} | GCCTGGCGGATCTCTGAGACTGTCTTGTGCCGCCTCCGG |
| | | variable | | CTTCACCTTCTCCAGATACGCTATGTCCTGGGTCCGACAG |
| | | region | | GCTCCTGGCAAAGGACTGGAATGGGTGTCCTCCATCTCT |
| | | | | GCCTCTGGCGCCACCACCTTTTACGCCGATCCTGTGAAG |
| | | | | GGCAGATTCACCATCAGCCGGGACAACTCCAAGAAAAC |
| | | | | CCTGTACCTGCAGATGAACTCCCTGAGAGCCGAGGACA |
| | | | | CCGCCGTGTACTACTGTGCCAAGGACCAGGACTTCGACA |
| | | | | TCCTGACCGGCTACCTGAATTGGTTCGACCCTTGGGGCC |
| | | | | AGGGCACACTGGTTACAGTGTCTAGC |
| 29 | F05 | Full | Amino acid sequence | EVQLVESGGGLVQPGGSLRLSCAASGFTFSRYAMSWVRQ |
| | | heavy | heavy chain | APGKGLEWVSSISASGATTFYADPVKGRFTISRDNSKKTLYL |
| | | chain | | QMNSLRAEDTAVYYCAKDQDFDILTGYLNWFDPWGQGT |
| | | sequence | | LVTVSSASTKGPSVFPLAPSSKSTSGGTAALGCLVKDYFPEP |
| | | | | VTVSWNSGALTSGVHTFPAVLQSSGLYSLSSVVTVPSSSLG |
| | | | | TQTYICNVNHKPSNTKVDKKVEPKSCDKTHTCPPCPAPELL |
| | | | | GGPSVFLFPPKPKDTLMISRTPEVTCVVVDVSHEDPEVKEN |
| | | | | WYVDGVEVHNAKTKPREEQYNSTYRVVSVLTVLHQDWL |
| | | | | NGKEYKCKVSNKALPAPIEKTISKAKGQPREPQVYTLPPSRD |
| | | | | ELTKNQVSLTCLVKGFYPSDIAVEWESNGQPENNYKTTPP |
| | | | | VLDSDGSFFLYSKLTVDKSRWQQGNVFSCSVMHGALHNH |
| | | | | YTQKSLSLSPG |
| 30 | F05 | Full | Nucleic acid sequence | GAAGTGCAGCTGGTTGAATCTGGCGGCGGATTGGTTCA |
| | | heavy | heavy chain | GCCTGGCGGATCTCTGAGACTGTCTTGTGCCGCCTCCGG |
| | | chain | | CTTCACCTTCTCCAGATACGCTATGTCCTGGGTCCGACAG |
| | | sequence | | GCTCCTGGCAAAGGACTGGAATGGGTGTCCTCCATCTCT |
| | | | | GCCTCTGGCGCCACCACCTTTTACGCCGATCCTGTGAAG |
| | | | | GGCAGATTCACCATCAGCCGGGACAACTCCAAGAAAAC |
| | | | | CCTGTACCTGCAGATGAACTCCCTGAGAGCCGAGGACA |
| | | | | CCGCCGTGTACTACTGTGCCAAGGACCAGGACTTCGACA |
| | | | | TCCTGACCGGCTACCTGAATTGGTTCGACCCTTGGGGCC |
| | | | | AGGGCACACTGGTTACAGTGTCTAGCGCCAGCACCAAG |
| | | | | GGCCCCTCTGTGTTCCCTCTGGCCCCTTCCAGCAAGTCCA |
| | | | | CCTCTGGCGGAACAGCCGCTCTGGGCTGCCTCGTGAAG |
| | | | | GACTACTTCCCCGAGCCTGTGACCGTGTCCTGGAACTCT |
| | | | | GGCGCTCTGACCAGCGGAGTGCACACCTTCCCTGCTGTG |
| | | | | CTGCAGTCCTCCGGCCTGTACTCCCTGTCCTCCGTCGTGA |
| | | | | CCGTGCCTTCCAGCTCTCTGGGCACCCAGACCTACATCT |
| | | | | GCAACGTGAACCACAAGCCCTCCAACACCAAGGTGGAC |
| | | | | AAGAAGGTGGAACCCAAGTCCTGCGACAAGACCCACAC |
| | | | | CTGTCCCCCTTGTCCTGCCCCTGAACTGCTGGGCGGACC |
| | | | | TTCCGTGTTCCTGTTCCCCCCAAAGCCCAAGGACACCCTG |
| | | | | ATGATCTCCCGGACCCCCGAAGTGACCTGCGTGGTGGT |
| | | | | GGATGTGTCCCACGAGGACCCTGAAGTGAAGTTCAATT |
| | | | | GGTACGTGGACGGCGTGGAAGTGCACAACGCCAAGACC |
| | | | | AAGCCTAGAGAGGAACAGTACAACTCCACCTACCGGGT |
| | | | | GGTGTCCGTGCTGACCGTGCTGCACCAGGATTGGCTGA |
| | | | | ACGGCAAAGAGTACAAGTGCAAGGTGTCCAACAAGGCC |
| | | | | CTGCCTGCCCCCATCGAAAAGACCATCTCCAAGGCCAAG |
| | | | | GGCCAGCCCCGGGAACCCCAGGTGTACACACTGCCCCCT |
| | | | | AGCAGGGACGAGCTGACCAAGAACCAGGTGTCCCTGAC |
| | | | | CTGTCTCGTGAAAGGCTTCTACCCCTCCGATATCGCCGT |
| | | | | GGAATGGGAGTCCAACGGCCAGCCTGAGAACAACTACA |
| | | | | AGACCACCCCCCCTGTGCTGGACTCCGACGGCTCATTCT |
| | | | | TCCTGTACAGCAAGCTGACAGTGGACAAGTCCCGGTGG |
| | | | | CAGCAGGGCAACGTGTTCTCCTGCTCCGTGATGCACGGC |
| | | | | GCCCTGCACAACCACTACACCCAGAAGTCCCTGTCCCTG |
| | | | | AGCCCCGGC |
| 31 | F05 | CDRL1 | Amino acid sequence | QSVSSY |
| | | (IMGT) | of CDRL1 using IMGT | |

| | | | | |
|---|---|---|---|---|
| 32 | F05 | CDRL2 | Amino acid sequence | DTS |
| | | (IMGT) | of CDRL2 using IMGT | |
| 33 | F05 | CDRL3 | Amino acid sequence | QQRRNWPLT |
| | | (IMGT) | of CDRL3 using IMGT | |
| 34 | F05 | CDRL1 | Amino acid sequence | RVSQSVSSYLA |
| | | (KABAT) | of CDRL1 using KABAT | |
| 35 | F05 | CDRL2 | Amino acid sequence | DTSNRAT |
| | | (KABAT) | of CDRL2 using KABAT | |
| 36 | F05 | CDRL3 | Amino acid sequence | QQRRNWPLT |
| | | (KABAT) | of CDRL3 using KABAT | |
| 37 | F05 | Light | Amino acid sequence | EIVLTQSPATLSLSPGERVILSCRVSQSVSSYLAWYQQKPGQ |
| | | chain | of V_{L} | APRLLIYDTSNRATGIPARFSGSGSGTDFTLTISSLEPEDFAV |
| | | variable | | YYCQQRRNWPLTFGGGTKVEIK |
| | | region | | |
| 38 | F05 | Light | Nucleic acid sequence | GAGATCGTGCTGACCCAGTCTCCTGCCACACTGTCACTG |
| | | chain | of V_{L} | TCTCCAGGCGAGAGAGTGATCCTGTCTTGCCGGGTGTC |
| | | variable | | CCAGTCCGTGTCCTCTTACCTGGCTTGGTATCAGCAGAA |
| | | region | | GCCCGGCCAGGCTCCTAGACTGCTGATCTACGACACCT |
| | | | | CCAACAGAGCCACAGGCATCCCCGCCAGATTTTCCGGC |
| | | | | TCTGGCTCTGGCACAGACTTTACCCTGACCATCTCCAGC |
| | | | | CTGGAACCTGAGGACTTCGCCGTGTACTACTGCCAGCA |
| | | | | GCGGAGAAACTGGCCCCTGACATTTGGCGGCGGAACA |
| | | | | AAGGTGGAAATCAAG |
| 39 | F05 | Full | Amino acid sequence | EIVLTQSPATLSLSPGERVILSCRVSQSVSSYLAWYQQKPGQ |
| | | light | light chain | APRLLIYDTSNRATGIPARFSGSGSGTDFTLTISSLEPEDFAV |
| | | chain | | YYCQQRRNWPLTFGGGTKVEIKRTVAAPSVFIFPPSDEQLK |
| | | sequence | | SGTASVVCLLNNFYPREAKVQWKVDNALQSGNSQESVTE |
| | | | | QDSKDSTYSLSSTLTLSKADYEKHKVYACEVTHQGLSSPVTK |
| | | | | SFNRGEC |
| 40 | F05 | Full | Nucleic acid sequence | GAGATCGTGCTGACCCAGTCTCCTGCCACACTGTCACTG |
| | | light | light chain | TCTCCAGGCGAGAGAGTGATCCTGTCTTGCCGGGTGTC |
| | | chain | | CCAGTCCGTGTCCTCTTACCTGGCTTGGTATCAGCAGAA |
| | | sequence | | GCCCGGCCAGGCTCCTAGACTGCTGATCTACGACACCT |
| | | | | CCAACAGAGCCACAGGCATCCCCGCCAGATTTTCCGGC |
| | | | | TCTGGCTCTGGCACAGACTTTACCCTGACCATCTCCAGC |
| | | | | CTGGAACCTGAGGACTTCGCCGTGTACTACTGCCAGCA |
| | | | | GCGGAGAAACTGGCCCCTGACATTTGGCGGCGGAACA |
| | | | | AAGGTGGAAATCAAGCGTACGGTGGCCGCTCCCTCCGT |
| | | | | GTTCATCTTCCCACCTTCCGACGAGCAGCTGAAGTCCGG |
| | | | | CACCGCTTCTGTCGTGTGCCTGCTGAACAACTTCTACCC |
| | | | | CCGCGAGGCCAAGGTGCAGTGGAAGGTGGACAACGCC |
| | | | | CTGCAGTCCGGCAACTCCCAGGAATCCGTGACCGAGCA |
| | | | | GGACTCCAAGGACAGCACCTACTCCCTGTCCTCCACCCT |
| | | | | GACCCTGTCCAAGGCCGACTACGAGAAGCACAAGGTGT |
| | | | | ACGCCTGCGAAGTGACCCACCAGGGCCTGTCTAGCCCC |
| | | | | GTGACCAAGTCTTTCAACCGGGGCGAGTGT |
| 41 | C02 | CDRH1 | Amino acid sequence | GLTFNNYA |
| | | (IMGT) | of CDRH1 using IMGT | |
| 42 | C02 | CDRH2 | Amino acid sequence | ISGSGGST |
| | | (IMGT) | of CDRH2 using IMGT | |
| 43 | C02 | CDRH3 | Amino acid sequence | AKGGLLYFGEFHFDY |
| | | (IMGT) | of CDRH3 using IMGT | |
| 44 | C02 | CDRH1 | Amino acid sequence | NNYA |
| | | (KABAT) | of CDRH1 using Kabat | |
| 45 | C02 | CDRH2 | Amino acid sequence | TISGSGGSTYYADSAKG |
| | | (KABAT) | of CDRH2 using Kabat | |
| 46 | C02 | CDRH3 | Amino acid sequence | GGLLYFGEFHFDY |
| | | (KABAT) | of CDRH3 using Kabat | |
| 47 | C02 | Heavy | Amino acid sequence | EVQLVESGGGLVQLGGSLRLSCAASGLTFNNYAMSWVRQ |
| | | chain | of V_{H} | APGKGLEWVSTISGSGGSTYYADSAKGRFTISRDNSKNTLY |
| | | variable | | LQMNSLRAEDTAVYYCAKGGLLYFGEFHFDYWGQGTLVT |
| | | region | | VSS |
| 48 | C02 | Heavy | Nucleotide sequence | GAGGTGCAGCTGGTGGAGTCTGGGGGAGGCTTGGTAC |
| | | chain | of V_{H} | AGCTTGGGGGGTCCCTGAGACTCTCCTGTGCAGCCTCT |
| | | variable | | GGACTCACCTTTAACAACTATGCCATGAGCTGGGTCCG |
| | | region | | CCAGGCTCCAGGGAAGGGGCTGGAGTGGGTCTCAACT |
| | | | | ATTAGTGGAAGTGGTGGCAGCACATACTACGCAGACTC |
| | | | | CGCGAAGGGCCGGTTCACCATCTCCAGAGACAATTCCA |
| | | | | AGAACACGCTGTATCTGCAAATGAACAGCCTGAGAGCC |
| | | | | GAGGACACGGCCGTATATTACTGTGCGAAAGGGGGCT |
| | | | | TACTATACTTCGGGGAGTTCCACTTTGACTACTGGGGCC |
| | | | | AGGGAACCCTGGTCACCGTCTCCTCA |
| 49 | C02 | Full | Amino acid sequence | EVQLVESGGGLVQLGGSLRLSCAASGLTFNNYAMSWVRQ |
| | | heavy | heavy chain | APGKGLEWVSTISGSGGSTYYADSAKGRFTISRDNSKNTLY |
| | | chain | | LQMNSLRAEDTAVYYCAKGGLLYFGEFHFDYWGQGTLVT |
| | | sequence | | VSSASTKGPSVFPLAPSSKSTSGGTAALGCLVKDYFPEPVTV |
| | | | | SWNSGALTSGVHTFPAVLQSSGLYSLSSVVTVPSSSLGTQT |
| | | | | YICNVNHKPSNTKVDKKVEPKSCDKTHTCPPCPAPELLGGP |
| | | | | SVFLFPPKPKDTLMISRTPEVTCVVVDVSHEDPEVKFNWYV |
| | | | | DGVEVHNAKTKPREEQYNSTYRVVSVLTVLHQDWLNGKE |
| | | | | YKCKVSNKALPAPIEKTISKAKGQPREPQVYTLPPSRDELTK |
| | | | | NQVSLTCLVKGFYPSDIAVEWESNGQPENNYKTTPPVLDS |
| | | | | DGSFFLYSKLTVDKSRWQQGNVFSCSVMHGALHNHYTQK |
| | | | | SLSLSPG |
| 50 | C02 | Full | Nucleic acid sequence | GAGGTGCAGCTGGTGGAGTCTGGGGGAGGCTTGGTAC |
| | | heavy | heavy chain | AGCTTGGGGGGTCCCTGAGACTCTCCTGTGCAGCCTCT |
| | | chain | | GGACTCACCTTTAACAACTATGCCATGAGCTGGGTCCG |
| | | sequence | | CCAGGCTCCAGGGAAGGGGCTGGAGTGGGTCTCAACT |
| | | | | ATTAGTGGAAGTGGTGGCAGCACATACTACGCAGACTC |
| | | | | CGCGAAGGGCCGGTTCACCATCTCCAGAGACAATTCCA |
| | | | | AGAACACGCTGTATCTGCAAATGAACAGCCTGAGAGCC |
| | | | | GAGGACACGGCCGTATATTACTGTGCGAAAGGGGGCT |
| | | | | TACTATACTTCGGGGAGTTCCACTTTGACTACTGGGGCC |
| | | | | AGGGAACCCTGGTCACCGTCTCCTCAGCCAGCACCAAG |
| | | | | GGCCCCTCTGTGTTCCCTCTGGCCCCTTCCAGCAAGTCC |
| | | | | ACCTCTGGCGGAACAGCCGCTCTGGGCTGCCTCGTGAA |
| | | | | GGACTACTTCCCCGAGCCTGTGACCGTGTCCTGGAACTC |
| | | | | TGGCGCTCTGACCAGCGGAGTGCACACCTTCCCTGCTG |
| | | | | TGCTGCAGTCCTCCGGCCTGTACTCCCTGTCCTCCGTCG |
| | | | | TGACCGTGCCTTCCAGCTCTCTGGGCACCCAGACCTACA |
| | | | | TCTGCAACGTGAACCACAAGCCCTCCAACACCAAGGTG |
| | | | | GACAAGAAGGTGGAACCCAAGTCCTGCGACAAGACCC |
| | | | | ACACCTGTCCCCCTTGTCCTGCCCCTGAACTGCTGGGCG |
| | | | | GACCTTCCGTGTTCCTGTTCCCCCCAAAGCCCAAGGACA |
| | | | | CCCTGATGATCTCCCGGACCCCCGAAGTGACCTGCGTG |
| | | | | GTGGTGGATGTGTCCCACGAGGACCCTGAAGTGAAGTT |
| | | | | CAATTGGTACGTGGACGGCGTGGAAGTGCACAACGCC |
| | | | | AAGACCAAGCCTAGAGAGGAACAGTACAACTCCACCTA |
| | | | | CCGGGTGGTGTCCGTGCTGACCGTGCTGCACCAGGATT |
| | | | | GGCTGAACGGCAAAGAGTACAAGTGCAAGGTGTCCAA |
| | | | | CAAGGCCCTGCCTGCCCCCATCGAAAAGACCATCTCCA |
| | | | | AGGCCAAGGGCCAGCCCCGGGAACCCCAGGTGTACAC |
| | | | | ACTGCCCCCTAGCAGGGACGAGCTGACCAAGAACCAG |
| | | | | GTGTCCCTGACCTGTCTCGTGAAAGGCTTCTACCCCTCC |
| | | | | GATATCGCCGTGGAATGGGAGTCCAACGGCCAGCCTG |
| | | | | AGAACAACTACAAGACCACCCCCCCTGTGCTGGACTCC |
| | | | | GACGGCTCATTCTTCCTGTACAGCAAGCTGACAGTGGA |
| | | | | CAAGTCCCGGTGGCAGCAGGGCAACGTGTTCTCCTGCT |
| | | | | CCGTGATGCACGGCGCCCTGCACAACCACTACACCCAG |
| | | | | AAGTCCCTGTCCCTGAGCCCCGGC |
| 51 | C02 | CDRL1 | Amino acid sequence | QGISNY |
| | | (IMGT) | of CDRL1 using IMGT | |
| 52 | C02 | CDRL2 | Amino acid sequence | AAS |
| | | (IMGT) | of CDRL2 using IMGT | |
| 53 | C02 | CDRL3 | Amino acid sequence | QHYNSYPLT |
| | | (IMGT) | of CDRL3 using IMGT | |
| 54 | C02 | CDRL1 | Amino acid sequence | RASQGISNYLA |
| | | (KABAT) | of CDRL1 using KABAT | |
| 55 | C02 | CDRL2 | Amino acid sequence | AASSLQS |
| | | (KABAT) | of CDRL2 using KABAT | |
| 56 | C02 | CDRL3 | Amino acid sequence | QHYNSYPLT |
| | | (KABAT) | of CDRL3 using KABAT | |
| 57 | C02 | Light | Amino acid sequence | DIQMTQSPSSLSASVGDRVTIICRASQGISNYLAWFQQKPG |
| | | chain | of V_{L} | KAPKSLIYAASSLQSGVPSKFSGSGSGTDFTLTISSLQPEDFA |
| | | variable | | TYYCQHYNSYPLTFGGGTKVEIK |
| | | region | | |
| 58 | C02 | Light | Nucleic acid sequence | GACATCCAGATGACCCAGTCTCCATCCTCACTGTCTGCA |
| | | chain | of V_{L} | TCTGTAGGAGACAGAGTCACCATCATTTGTCGGGCGAG |
| | | variable | | TCAGGGCATTAGCAATTATTTAGCCTGGTTTCAGCAGAA |
| | | region | | ACCAGGGAAAGCCCCTAAGTCCCTGATCTATGCTGCAT |
| | | | | CCAGTTTGCAAAGTGGGGTCCCATCAAAGTTCAGCGGC |
| | | | | AGTGGATCTGGGACAGATTTCACTCTCACCATCAGCAG |
| | | | | CCTGCAGCCTGAAGATTTTGCAACTTATTACTGCCAACA |
| | | | | TTATAATAGTTACCCGCTCACTTTCGGCGGAGGGACCA |
| | | | | AGGTGGAGATCAAA |
| 59 | C02 | Full | Amino acid sequence | DIQMTQSPSSLSASVGDRVTIICRASQGISNYLAWFQQKPG |
| | | light | light chain | KAPKSLIYAASSLQSGVPSKFSGSGSGTDFTLTISSLQPEDFA |
| | | chain | | TYYCQHYNSYPLTFGGGTKVEIKRTVAAPSVFIFPPSDEQLK |
| | | sequence | | SGTASVVCLLNNFYPREAKVQWKVDNALQSGNSQESVTE |
| | | | | QDSKDSTYSLSSTLTLSKADYEKHKVYACEVTHQGLSSPVTK |
| | | | | SFNRGEC |
| 60 | C02 | Full | Nucleic acid sequence | GACATCCAGATGACCCAGTCTCCATCCTCACTGTCTGCA |
| | | light | light chain | TCTGTAGGAGACAGAGTCACCATCATTTGTCGGGCGAG |
| | | chain | | TCAGGGCATTAGCAATTATTTAGCCTGGTTTCAGCAGAA |
| | | sequence | | ACCAGGGAAAGCCCCTAAGTCCCTGATCTATGCTGCAT |
| | | | | CCAGTTTGCAAAGTGGGGTCCCATCAAAGTTCAGCGGC |
| | | | | AGTGGATCTGGGACAGATTTCACTCTCACCATCAGCAG |
| | | | | CCTGCAGCCTGAAGATTTTGCAACTTATTACTGCCAACA |
| | | | | TTATAATAGTTACCCGCTCACTTTCGGCGGAGGGACCA |
| | | | | AGGTGGAGATCAAACGTACGGTGGCCGCTCCCTCCGTG |
| | | | | TTCATCTTCCCACCTTCCGACGAGCAGCTGAAGTCCGGC |
| | | | | ACCGCTTCTGTCGTGTGCCTGCTGAACAACTTCTACCCC |
| | | | | CGCGAGGCCAAGGTGCAGTGGAAGGTGGACAACGCCC |
| | | | | TGCAGTCCGGCAACTCCCAGGAATCCGTGACCGAGCAG |
| | | | | GACTCCAAGGACAGCACCTACTCCCTGTCCTCCACCCTG |
| | | | | ACCCTGTCCAAGGCCGACTACGAGAAGCACAAGGTGTA |
| | | | | CGCCTGCGAAGTGACCCACCAGGGCCTGTCTAGCCCCG |
| | | | | TGACCAAGTCTTTCAACCGGGGCGAGTGT |
| 61 | E04 | CDRH1 | Amino acid sequence | GVTFSNAW |
| | | (IMGT) | of CDRH1 using IMGT | |
| 62 | E04 | CDRH2 | Amino acid sequence | IKSKTDGGTT |
| | | (IMGT) | of CDRH2 using IMGT | |
| 63 | E04 | CDRH3 | Amino acid sequence | TIEAVAGHFDY |
| | | (IMGT) | of CDRH3 using IMGT | |
| 64 | E04 | CDRH1 | Amino acid sequence | SNAWMS |
| | | (KABAT) | of CDRH1 using Kabat | |
| 65 | E04 | CDRH2 | Amino acid sequence | RIKSKTDGGTTDYAAPVKG |
| | | (KABAT) | of CDRH2 using Kabat | |
| 66 | E04 | CDRH3 | Amino acid sequence | EAVAGHFDY |
| | | (KABAT) | of CDRH3 using Kabat | |
| 67 | E04 | Heavy | Amino acid sequence | EVQLVESGGGLVKPGGSPRLSCAAYGVTFSNAWMSWVR |
| | | chain | of V_{H} | QAPGKGLEWVGRIKSKTDGGTTDYAAPVKGRFTISRDDSK |
| | | variable | | NTLYLQMNSLKTEDTAVYYCTIEAVAGHFDYWGQGTLVTV |
| | | region | | SS |
| 68 | E04 | Heavy | Nucleotide sequence | GAGGTGCAGTTGGTGGAGTCTGGGGGAGGCTTGGTAA |
| | | chain | of V_{H} | AGCCTGGGGGGTCCCCTAGACTCTCCTGTGCAGCCTAT |
| | | variable | | GGAGTCACTTTCAGTAACGCCTGGATGAGTTGGGTCCG |
| | | region | | CCAGGCTCCAGGGAAGGGGCTGGAGTGGGTTGGCCGT |
| | | | | ATTAAAAGCAAAACTGATGGTGGGACAACAGACTATGC |
| | | | | TGCACCCGTGAAAGGCAGATTCACCATCTCAAGAGATG |
| | | | | ATTCAAAAAACACGCTGTATCTGCAAATGAACAGCCTG |
| | | | | AAAACCGAGGACACAGCCGTGTATTACTGTACCATAGA |
| | | | | AGCAGTGGCTGGTCACTTTGACTACTGGGGCCAGGGAA |
| | | | | CCCTGGTCACCGTCTCCTCA |
| 69 | E04 | Full | Amino acid sequence | EVQLVESGGGLVKPGGSPRLSCAAYGVTFSNAWMSWVR |
| | | heavy | heavy chain | QAPGKGLEWVGRIKSKTDGGTTDYAAPVKGRFTISRDDSK |
| | | chain | | NTLYLQMNSLKTEDTAVYYCTIEAVAGHFDYWGQGTLVTV |
| | | sequence | | SSASTKGPSVFPLAPSSKSTSGGTAALGCLVKDYFPEPVTVS |
| | | | | WNSGALTSGVHTFPAVLQSSGLYSLSSVVTVPSSSLGTQTYI |
| | | | | CNVNHKPSNTKVDKKVEPKSCDKTHTCPPCPAPELLGGPS |
| | | | | VFLFPPKPKDTLMISRTPEVTCVVVDVSHEDPEVKFNWYV |
| | | | | DGVEVHNAKTKPREEQYNSTYRVVSVLTVLHQDWLNGKE |
| | | | | YKCKVSNKALPAPIEKTISKAKGQPREPQVYTLPPSRDELTK |
| | | | | NQVSLTCLVKGFYPSDIAVEWESNGQPENNYKTTPPVLDS |
| | | | | DGSFFLYSKLTVDKSRWQQGNVFSCSVMHGALHNHYTQK |
| | | | | SLSLSPG |
| 70 | E04 | Full | Nucleic acid sequence | GAGGTGCAGTTGGTGGAGTCTGGGGGAGGCTTGGTAA |
| | | heavy | heavy chain | AGCCTGGGGGGTCCCCTAGACTCTCCTGTGCAGCCTAT |
| | | chain | | GGAGTCACTTTCAGTAACGCCTGGATGAGTTGGGTCCG |
| | | sequence | | CCAGGCTCCAGGGAAGGGGCTGGAGTGGGTTGGCCGT |
| | | | | ATTAAAAGCAAAACTGATGGTGGGACAACAGACTATGC |
| | | | | TGCACCCGTGAAAGGCAGATTCACCATCTCAAGAGATG |
| | | | | ATTCAAAAAACACGCTGTATCTGCAAATGAACAGCCTG |
| | | | | AAAACCGAGGACACAGCCGTGTATTACTGTACCATAGA |
| | | | | AGCAGTGGCTGGTCACTTTGACTACTGGGGCCAGGGAA |
| | | | | CCCTGGTCACCGTCTCCTCAGCCAGCACCAAGGGCCCCT |
| | | | | CTGTGTTCCCTCTGGCCCCTTCCAGCAAGTCCACCTCTG |
| | | | | GCGGAACAGCCGCTCTGGGCTGCCTCGTGAAGGACTAC |
| | | | | TTCCCCGAGCCTGTGACCGTGTCCTGGAACTCTGGCGCT |
| | | | | CTGACCAGCGGAGTGCACACCTTCCCTGCTGTGCTGCA |
| | | | | GTCCTCCGGCCTGTACTCCCTGTCCTCCGTCGTGACCGT |
| | | | | GCCTTCCAGCTCTCTGGGCACCCAGACCTACATCTGCAA |
| | | | | CGTGAACCACAAGCCCTCCAACACCAAGGTGGACAAGA |
| | | | | AGGTGGAACCCAAGTCCTGCGACAAGACCCACACCTGT |
| | | | | CCCCCTTGTCCTGCCCCTGAACTGCTGGGCGGACCTTCC |
| | | | | GTGTTCCTGTTCCCCCCAAAGCCCAAGGACACCCTGATG |
| | | | | ATCTCCCGGACCCCCGAAGTGACCTGCGTGGTGGTGGA |
| | | | | TGTGTCCCACGAGGACCCTGAAGTGAAGTTCAATTGGT |
| | | | | ACGTGGACGGCGTGGAAGTGCACAACGCCAAGACCAA |
| | | | | GCCTAGAGAGGAACAGTACAACTCCACCTACCGGGTGG |
| | | | | TGTCCGTGCTGACCGTGCTGCACCAGGATTGGCTGAAC |
| | | | | GGCAAAGAGTACAAGTGCAAGGTGTCCAACAAGGCCC |
| | | | | TGCCTGCCCCCATCGAAAAGACCATCTCCAAGGCCAAG |
| | | | | GGCCAGCCCCGGGAACCCCAGGTGTACACACTGCCCCC |
| | | | | TAGCAGGGACGAGCTGACCAAGAACCAGGTGTCCCTG |
| | | | | ACCTGTCTCGTGAAAGGCTTCTACCCCTCCGATATCGCC |
| | | | | GTGGAATGGGAGTCCAACGGCCAGCCTGAGAACAACT |
| | | | | ACAAGACCACCCCCCCTGTGCTGGACTCCGACGGCTCA |
| | | | | TTCTTCCTGTACAGCAAGCTGACAGTGGACAAGTCCCG |
| | | | | GTGGCAGCAGGGCAACGTGTTCTCCTGCTCCGTGATGC |
| | | | | ACGGCGCCCTGCACAACCACTACACCCAGAAGTCCCTG |
| | | | | TCCCTGAGCCCCGGC |
| 71 | E04 | CDRL1 | Amino acid sequence | QSISSW |
| | | (IMGT) | of CDRL1 using IMGT | |
| 72 | E04 | CDRL2 | Amino acid sequence | KAS |
| | | (IMGT) | of CDRL2 using IMGT | |
| 73 | E04 | CDRL3 | Amino acid sequence | QQYNNYSPT |
| | | (IMGT) | of CDRL3 using IMGT | |
| 74 | E04 | CDRL1 | Amino acid sequence | RASQSISSWLA |
| | | (KABAT) | of CDRL1 using KABAT | |
| 75 | E04 | CDRL2 | Amino acid sequence | KASSLES |
| | | (KABAT) | of CDRL2 using KABAT | |
| 76 | E04 | CDRL3 | Amino acid sequence | QQYNNYSPT |
| | | (KABAT) | of CDRL3 using KABAT | |
| 77 | E04 | Light | Amino acid sequence | DIQMTQSPSTLSASVGDRVTITCRASQSISSWLAWYQQKP |
| | | chain | of V_{L} | GKAPKLLIYKASSLESGVPSRFSGSGSGTEFTLTISSLQPDDF |
| | | variable | | ATYYCQQYNNYSPTFGQGTKVEIK |
| | | region | | |
| 78 | E04 | Light | Nucleic acid sequence | GACATCCAGATGACCCAGTCTCCTTCCACCCTGTCTGCA |
| | | chain | of V_{L} | TCTGTAGGAGACAGAGTCACCATCACTTGCCGGGCCAG |
| | | variable | | TCAGAGTATTAGTAGCTGGTTGGCCTGGTATCAGCAGA |
| | | region | | AACCAGGGAAAGCCCCTAAGCTCCTGATCTATAAGGCG |
| | | | | TCTAGTTTAGAAAGTGGGGTCCCATCAAGGTTCAGCGG |
| | | | | CAGTGGATCTGGGACAGAATTCACTCTCACCATCAGCA |
| | | | | GCCTGCAGCCTGATGATTTTGCAACTTATTACTGCCAAC |
| | | | | AGTATAATAATTATTCTCCGACGTTCGGCCAGGGGACC |
| | | | | AAGGTGGAAATCAAA |
| 79 | E04 | Full | Amino acid sequence | DIQMTQSPSTLSASVGDRVTITCRASQSISSWLAWYQQKP |
| | | light | light chain | GKAPKLLIYKASSLESGVPSRFSGSGSGTEFTLTISSLQPDDF |
| | | chain | | ATYYCQQYNNYSPTFGQGTKVEIKRTVAAPSVFIFPPSDEQ |
| | | sequence | | LKSGTASVVCLLNNFYPREAKVQWKVDNALQSGNSQESV |
| | | | | TEQDSKDSTYSLSSTLTLSKADYEKHKVYACEVTHQGLSSPV |
| | | | | TKSFNRGEC |
| 80 | E04 | Full | Nucleic acid sequence | GACATCCAGATGACCCAGTCTCCTTCCACCCTGTCTGCA |
| | | light | light chain | TCTGTAGGAGACAGAGTCACCATCACTTGCCGGGCCAG |
| | | chain | | TCAGAGTATTAGTAGCTGGTTGGCCTGGTATCAGCAGA |
| | | sequence | | AACCAGGGAAAGCCCCTAAGCTCCTGATCTATAAGGCG |
| | | | | TCTAGTTTAGAAAGTGGGGTCCCATCAAGGTTCAGCGG |
| | | | | CAGTGGATCTGGGACAGAATTCACTCTCACCATCAGCA |
| | | | | GCCTGCAGCCTGATGATTTTGCAACTTATTACTGCCAAC |
| | | | | AGTATAATAATTATTCTCCGACGTTCGGCCAGGGGACC |
| | | | | AAGGTGGAAATCAAACGTACGGTGGCCGCTCCCTCCGT |
| | | | | GTTCATCTTCCCACCTTCCGACGAGCAGCTGAAGTCCGG |
| | | | | CACCGCTTCTGTCGTGTGCCTGCTGAACAACTTCTACCC |
| | | | | CCGCGAGGCCAAGGTGCAGTGGAAGGTGGACAACGCC |
| | | | | CTGCAGTCCGGCAACTCCCAGGAATCCGTGACCGAGCA |
| | | | | GGACTCCAAGGACAGCACCTACTCCCTGTCCTCCACCCT |
| | | | | GACCCTGTCCAAGGCCGACTACGAGAAGCACAAGGTGT |
| | | | | ACGCCTGCGAAGTGACCCACCAGGGCCTGTCTAGCCCC |
| | | | | GTGACCAAGTCTTTCAACCGGGGCGAGTGT |
| 81 | Human | | Nucleic acid sequence | ATGGCCGGGCCTCCGAGGCTCCTGCTGCTGCCCCTGCTT |
| | CD7 | | | CTGGCGCTGGCTCGCGGCCTGCCTGGGGCCCTGGCTGC |
| | | | | CCAAGAGGTGCAGCAGTCTCCCCACTGCACGACTGTCCC |
| | | | | CGTGGGAGCCTCCGTCAACATCACCTGCTCCACCAGCGG |
| | | | | GGGCCTGCGTGGGATCTACCTGAGGCAGCTCGGGCCAC |
| | | | | AGCCCCAAGACATCATTTACTACGAGGACGGGGTGGTG |
| | | | | CCCACTACGGACAGACGGTTCCGGGGCCGCATCGACTTC |
| | | | | TCAGGGTCCCAGGACAACCTGACTATCACCATGCACCGC |
| | | | | CTGCAGCTGTCGGACACTGGCACCTACACCTGCCAGGCC |
| | | | | ATCACGGAGGTCAATGTCTACGGCTCCGGCACCCTGGTC |
| | | | | CTGGTGACAGAGGAACAGTCCCAAGGATGGCACAGATG |
| | | | | CTCGGACGCCCCACCAAGGGCCTCTGCCCTCCCTGCCCC |
| | | | | ACCGACAGGCTCCGCCCTCCCTGACCCGCAGACAGCCTC |
| | | | | TGCCCTCCCTGACCCGCCAGCAGCCTCTGCCCTCCCTGCG |
| | | | | GCCCTGGCGGTGATCTCCTTCCTCCTCGGGCTGGGCCTG |
| | | | | GGGGTGGCGTGTGTGCTGGCGAGGACACAGATAAAGA |
| | | | | AACTGTGCTCGTGGCGGGATAAGAATTCGGCGGCATGT |
| | | | | GTGGTGTACGAGGACATGTCGCACAGCCGCTGCAACAC |
| | | | | GCTGTCCTCCCCCAACCAGTACCAGTGA |
| 82 | Human | | Amino acid sequence | MAGPPRLLLLPLLLALARGLPGALAAQEVQQSPHCTTVPV |
| | CD7 | | | GASVNITCSTSGGLRGIYLRQLGPQPQDIIYYEDGVVPTTDR |
| | | | | RFRGRIDFSGSQDNLTITMHRLQLSDTGTYTCQAITEVNVY |
| | | | | GSGTLVLVTEEQSQGWHRCSDAPPRASALPAPPTGSALPD |
| | | | | PQTASALPDPPAASALPAALAVISFLLGLGLGVACVLARTQI |
| | | | | KKLCSWRDKNSAACVVYEDMSHSRCNTLSSPNQ |
| 83 | Cynomolgus | | Nucleic acid sequence | ATGGCCGGGCTCCCGAGGCTCCTGCTGCTGCCCCTCCTC |
| | monkey | | | CTGGCGCTGGCTCGTGGCCTGCCTGGGGCCCTGGCCGC |
| | (Macaca | | | CCAAGAGGTGCAGCAGTCTCCCCACTGCACGATTGCCCC |
| | fascicularis) | | | CGTGGGAGGCTCCGTCAACATCACCTGCTCCACCAGCGG |
| | CD7 | | | GGAACTGCATGGGATCTACCTGAGGCAGCTCGGGCCAC |
| | | | | AGCCCCAAAACATCATTTACTACGAGGACAGGGTGGTG |
| | | | | CCCACTACGGACAAACGGTTCCAGGGCCGCATTGACTTC |
| | | | | TCGGGGTCCCAGGACAACCTGACTATTACCATGCACCAC |
| | | | | CTGCAGCCGTCGGACACTGGCACCTACACCTGCCAGGCC |
| | | | | GTCACGGAGATCAATGTCTACGGCTCCGGTACCCTGGTC |
| | | | | CTGGTGACAGAGGAACAGTCCCAAGGATTGCACAGATG |
| | | | | CTCGGACGCCCCGCCGACAGGCTCTGCCCTCCCTGTCCC |
| | | | | GCCGACAACCTCTGCCCTCCCTGCCCTGCCAACAGCCTCT |
| | | | | GCCCTCCCTGCCCTGCCGACAGCCTCTGCCCTCCCTGTG |
| | | | | GCCCTGGCAGTGGTCTCCTTCCTCCTCGGGCTGGCCCTG |
| | | | | GGGGTGGCCTGTGTGCTGGTGAGGACGCAGATGAAGA |
| | | | | AACTGTGCTCGTCGCGGGATAAGAATTCGGCAGCGTGT |
| | | | | GTGGTGTACGAGGACATGTCCCGCAGCCGCTGCAACAC |
| | | | | GCTGTCCTCCCCCAACCAGTACCAGTGA |
| 84 | Cynomolgus | | Amino acid sequence | MAGLPRLLLLPLLLALARGLPGALAAQEVQQSPHCTIAPVG |
| | monkey | | | GSVNITCSTSGELHGIYLRQLGPQPQNIIYYEDRVVPTTDKR |
| | (Macaca | | | FQGRIDFSGSQDNLTITMHHLQPSDTGTYTCQAVTEINVY |
| | fascicularis) | | | GSGTLVLVTEEQSQGLHRCSDAPPTGSALPVPPTTSALPAL |
| | CD7 | | | PTASALPALPTASALPVALAVVSFLLGLALGVACVLVRTQM |
| | | | | KKLCSSRDKNSAACVVYEDMSRSRCNTLSSPNQYQ |
| 85 | Rat | | Nucleic acid sequence | ATGACTCAGGCACTGTTGGCTTTGCTACTTACACTGGCT |
| | (Rattus | | | AGAATCCTGCCTGGCCCCCTGGATGCCCAAGAGGTACAT |
| | norvegicus) | | | CAGTCTCCCCGAGTCGTGATCGCCTCCGAGGGAGAGTCC |
| | CD7 | | | ATCAACATCACCTGCTCTACAAGAGGGGATTTGGAAGG |
| | | | | GCTCATAATGAAGCGGATCTGGCCACAGGCTTCCAATGT |
| | | | | GATTTACTTTGAAGACGAATTGGAGCCCACAGTAGACA |
| | | | | GTGCCTTCTCAGGCCGAATTAATTTCTCTGGGTCCCAGA |
| | | | | AGAATCTGACCATCATCATGAGCCTTCTCCAGGAGGCAG |
| | | | | ACACTGGAGCCTACACCTGCGAGGCCGTCAGAAAAGTC |
| | | | | AGTGTCCATGGCTTGTTCACCACGGTTGTGGTGAAAGAA |
| | | | | AAACTATCCCATGAAGCGTACAGATCCCAGGAACCTCTG |
| | | | | CAGACATCAGTTTCCCTCCCAGCTGCCATTGCTGTAAGCT |
| | | | | TCTTCCTCACTGGGCTGATCCTTGGGGTGCTGTGCAGCA |
| | | | | TGCTGAGGAAGACGCAGATCAAGAAACTGTGTGCCTCA |
| | | | | GGGAATAAGGACTCTCTGTGCGTCGTGTATGAAGACAT |
| | | | | GTCCTGCAGCAACCGCAAGATACCATGTACCCCCAACCA |
| | | | | GTACCAATGA |
| 86 | Rat | | Amino acid sequence | MTQALLALLLTLARILPGPLDAQEVHQSPRVVIASEGESINIT |
| | (Rattus | | | CSTRGDLEGLIMKRIWPQASNVIYFEDELEPTVDSAFSGRIN |
| | norvegicus) | | | FSGSQKNLTIIMSLLQEADTGAYTCEAVRKVSVHGLFTTVV |
| | CD7 | | | VKEKLSHEAYRSQEPLQTSVSLPAAIAVSFFLTGLILGVLCSM |
| | | | | LRKTQIKKLCASGNKDSLCVVYEDMSCSNRKIPCTPNQYQ |
| 87 | Human | IGHG1*01 | Human Heavy Chain | gcctccaccaagggcccatcggtcttccccctggcaccctcctccaagagc |
| | IgG1 | | Constant Region | acctctgggggcacagcggccctgggctgcctggtcaaggactacttccc |
| | constant | | (IGHG1*01) | cgaaccggtgacggtgtcgtggaactcaggcgccctgaccagcggcgtgc |
| | region | | Nucleotide Sequence | acaccttcccggctgtcctacagtcctcaggactctactccctcagcagcgt |
| | | | | ggtgaccgtgccctccagcagcttgggcacccagacctacatctgcaacg |
| | | | | tgaatcacaagcccagcaacaccaaggtggacaagaaagttgagccca |
| | | | | aatcttgtgacaaaactcacacatgcccaccgtgcccagcacctgaactc |
| | | | | ctggggggaccgtcagtcttcctcttccccccaaaacccaaggacaccctc |
| | | | | atgatctcccggacccctgaggtcacatgcgtggtggtggacgtgagcca |
| | | | | cgaagaccctgaggtcaagttcaactggtacgtggacggcgtggaggtgc |
| | | | | ataatgccaagacaaagccgcgggaggagcagtacaacagcacgtacc |
| | | | | gggtggtcagcgtcctcaccgtcctgcaccaggactggctgaatggcaag |
| | | | | gagtacaagtgcaaggtctccaacaaagccctcccagcccccatcgaga |
| | | | | aaaccatctccaaagccaaagggcagccccgagaaccacaggtgtacac |
| | | | | cctgcccccatcccgggatgagctgaccaagaaccaggtcagcctgacct |
| | | | | gcctggtcaaaggcttctatcccagcgacatcgccgtggagtgggagagc |
| | | | | aatgggcagccggagaacaactacaagaccacgcctcccgtgctggact |
| | | | | ccgacggctccttcttcctctacagcaagctcaccgtggacaagagcaggt |
| | | | | ggcagcaggggaacgtcttctcatgctccgtgatgcatgaggctctgcaca |
| | | | | accactacacgcagaagagcctctccctgtctccgggtaaa |
| 88 | | | Human Heavy Chain | ASTKGPSVFPLAPSSKSTSGGTAALGCLVKDYFPEPVTVSW |
| | | | Constant Region | NSGALTSGVHTFPAVLQSSGLYSLSSVVTVPSSSLGTQTYIC |
| | | | (IGHG1*01) Protein | NVNHKPSNTKVDKKVEPKSCDKTHTCPPCPAPELLGGPSV |
| | | | Sequence (P01857) | FLFPPKPKDTLMISRTPEVTCVVVDVSHEDPEVKFNWYVD |
| | | | | GVEVHNAKTKPREEQYNSTYRVVSVLTVLHQDWLNGKEY |
| | | | | KCKVSNKALPAPIEKTISKAKGQPREPQVYTLPPSRDELTKN |
| | | | | QVSLTCLVKGFYPSDIAVEWESNGQPENNYKTTPPVLDSD |
| | | | | GSFFLYSKLTVDKSRWQQGNVFSCSVMHEALHNHYTQKS |
| | | | | LSLSPGK |
| 89 | Human | IGHG1*02 | Human Heavy Chain | gcctccaccaagggcccatcggtcttccccctggcaccctcctccaagagc |
| | IgG1 | or | Constant Region | acctctgggggcacagcggccctgggctgcctggtcaaggactacttccc |
| | constant | | (IGHG1*02 or | cgaaccggtgacggtgtcgtggaactcaggcgccctgaccagcggcgtgc |
| | region | | IGHG1*05) Nucleotide | acaccttcccggctgtcctacagtcctcaggactctactccctcagcagcgt |
| | | | Sequence | ggtgaccgtgccctccagcagcttgggcacccagacctacatctgcaacg |
| | | | | tgaatcacaagcccagcaacaccaaggtggacaagaaagttgagccca |
| | | | | aatcttgtgacaaaactcacacatgcccaccgtgcccagcacctgaactc |
| | | | | ctggggggaccgtcagtcttcctcttccccccaaaacccaaggacaccctc |
| | | | | atgatctcccggacccctgaggtcacatgcgtggtggtggacgtgagcca |
| | | | | cgaagaccctgaggtcaagttcaactggtacgtggacggcgtggaggtgc |
| | | | | ataatgccaagacaaagccgcgggaggagcagtacaacagcacgtacc |
| | | | | gtgtggtcagcgtcctcaccgtcctgcaccaggactggctgaatggcaag |
| | | | | gagtacaagtgcaaggtctccaacaaagccctcccagcccccatcgaga |
| | | | | aaaccatctccaaagccaaagggcagccccgagaaccacaggtgtacac |
| | | | | cctgcccccatcccgggatgagctgaccaagaaccaggtcagcctgacct |
| | | | | gcctggtcaaaggcttctatcccagcgacatcgccgtggagtgggagagc |
| | | | | aatgggcagccggagaacaactacaagaccacgcctcccgtgctggact |
| | | | | ccgacggctccttcttcctctacagcaagctcaccgtggacaagagcaggt |
| | | | | ggcagcaggggaacgtcttctcatgctccgtgatgcatgaggctctgcaca |
| | | | | accactacacgcagaagagcctctccctgtctccgggtaaa |
| 90 | | IGHG1*05 | Human Heavy Chain | A S T K G P S V F P L A P S S K S T S G G T A A |
| | | | | L G C L V K D Y F P E P V T V S W N S G A L T S G |
| | | | Constant Region | V H T F P A V L Q S S G L Y S L S S V V T |
| | | | (IGHG1*02) Protein | V P S S S L G T Q T Y I C N V N H K P S N T K V D |
| | | | Sequence | K K V E P K S C D K T H T C P P C P A P E L L G G P |
| | | | | S V F L F P P K P K D T L M I S R T P E V T C V |
| | | | | V V D V S H E D P E V K E N W Y V D G V E V H N A K |
| | | | | T K P R E E Q Y N S T Y R V V S V L T V LH Q D W |
| | | | | L N G K E Y K C K V S N K A L P A P I E K T I S K |
| | | | | K G Q P R E P Q V Y T L P P S R D E L T K N Q V S L |
| | | | | T C L V K G F Y P S D I A V E W E S N G Q P E N N |
| | | | | Y K T T P P V L D S D G S F F L Y S K L T V D K |
| | | | | S R W Q Q G N V F S C S V M H E A L H N H Y T Q |
| | | | | K S L S L S P G K |
| 91 | Human | IGHG1*03 | Human Heavy Chain | gcctccaccaagggcccatcggtcttccccctggcaccctcctccaagagc |
| | IgG1 | | Constant Region | acctctgggggcacagcggccctgggctgcctggtcaaggactacttccc |
| | constant | | (IGHG1*03) | cgaaccggtgacggtgtcgtggaactcaggcgccctgaccagcggcgtgc |
| | region | | Nucleotide Sequence | acaccttcccggctgtcctacagtcctcaggactctactccctcagcagcgt |
| | | | (Y14737) | ggtgaccgtgccctccagcagcttgggcacccagacctacatctgcaacg |
| | | | | tgaatcacaagcccagcaacaccaaggtggacaagagagttgagcccaa |
| | | | | atcttgtgacaaaactcacacatgcccaccgtgcccagcacctgaactcct |
| | | | | ggggggaccgtcagtcttcctcttccccccaaaacccaaggacaccctcat |
| | | | | gatctcccggacccctgaggtcacatgcgtggtggtggacgtgagccacg |
| | | | | aagaccctgaggtcaagttcaactggtacgtggacggcgtggaggtgcat |
| | | | | aatgccaagacaaagccgcgggaggagcagtacaacagcacgtaccgt |
| | | | | gtggtcagcgtcctcaccgtcctgcaccaggactggctgaatggcaagga |
| | | | | gtacaagtgcaaggtctccaacaaagccctcccagcccccatcgagaaa |
| | | | | accatctccaaagccaaagggcagccccgagaaccacaggtgtacaccc |
| | | | | tgcccccatcccgggaggagatgaccaagaaccaggtcagcctgacctg |
| | | | | cctggtcaaaggcttctatcccagcgacatcgccgtggagtgggagagca |
| | | | | atgggcagccggagaacaactacaagaccacgcctcccgtgctggactc |
| | | | | cgacggctccttcttcctctatagcaagctcaccgtggacaagagcaggtg |
| | | | | gcagcaggggaacgtcttctcatgctccgtgatgcatgaggctctgcaca |
| | | | | accactacacgcagaagagcctctccctgtccccgggtaaa |
| 92 | | | Human Heavy Chain | A S T K G P S V F P L A P S S K S T S G G T A A L |
| | | | Constant Region | G C L V K D Y F P E P V T V S W N S G A L T S G V H |
| | | | (IGHG1*03) Protein | T F P A V L Q S S G L Y S L S S V V T V P S S S |
| | | | Sequence | L G T Q T Y I C N V N H K P S N T K V D K R V E P |
| | | | | K S C D K T H T C P P C P A P E L L G G P S V F L |
| | | | | F P P K P K D T L M I S R T P E V T C V V V D V |
| | | | | S H E D P E V K E N W Y V D G V E V H N A K T K P |
| | | | | R E E Q Y N S T Y R V V S V L T V L H Q D W L N G K |
| | | | | E Y K C K V S N K A L P A P I E K T I S K A K G Q |
| | | | | P R E P Q V Y T L P P S R E E M T K N Q V S L T C L V |
| | | | | K G F Y P S D I A V E W E S N G Q P E N N Y K T T |
| | | | | P P V L D S D G S F F L Y S K L T V D K S R W |
| | | | | Q Q G N V F S C S V M H E A |
| | | | | L H N H Y T Q K S L S L S P G K |
| 93 | Human | IGHG1*04 | Human Heavy Chain | gcctccaccaagggcccatcggtcttccccctggcaccctcctccaagagc |
| | IgG1 | | Constant Region | acctctgggggcacagcggccctgggctgcctggtcaaggactacttccc |
| | constant | | (IGHG1*04) | cgaaccggtgacggtgtcgtggaactcaggcgccctgaccagcggcgtgc |
| | region | | Nucleotide Sequence | acaccttcccggctgtcctacagtcctcaggactctactccctcagcagcgt |
| | | | | ggtgaccgtgccctccagcagcttgggcacccagacctacatctgcaacg |
| | | | | tgaatcacaagcccagcaacaccaaggtggacaagaaagttgagccca |
| | | | | aatcttgtgacaaaactcacacatgcccaccgtgcccagcacctgaactc |
| | | | | ctggggggaccgtcagtcttcctcttccccccaaaacccaaggacaccctc |
| | | | | atgatctcccggacccctgaggtcacatgcgtggtggtggacgtgagcca |
| | | | | cgaagaccctgaggtcaagttcaactggtacgtggacggcgtggaggtgc |
| | | | | ataatgccaagacaaagccgcgggaggagcagtacaacagcacgtacc |
| | | | | gtgtggtcagcgtcctcaccgtcctgcaccaggactggctgaatggcaag |
| | | | | gagtacaagtgcaaggtctccaacaaagccctcccagcccccatcgaga |
| | | | | aaaccatctccaaagccaaagggcagccccgagaaccacaggtgtacac |
| | | | | cctgcccccatcccgggatgagctgaccaagaaccaggtcagcctgacct |
| | | | | gcctggtcaaaggcttctatcccagcgacatcgccgtggagtgggagagc |
| | | | | aatgggcagccggagaacaactacaagaccacgcctcccgtgctggact |
| | | | | ccgacggctccttcttcctctacagcaagctcaccgtggacaagagcaggt |
| | | | | ggcagcaggggaacatcttctcatgctccgtgatgcatgaggctctgcac |
| | | | | aaccactacacgcagaagagcctctccctgtctccgggtaaa |
| 94 | | | Human Heavy Chain | ASTKGPSVFPLAPSSKSTSGGTAALGCLVKDYFPEPVTVSW |
| | | | Constant Region | NSGALTSGVHTFPAVLQSSGLYSLSSVVTVPSSSLGTQTYIC |
| | | | (IGHG1*04) Protein | NVNHKPSNTKVDKKVEPKSCDKTHTCPPCPAPELLGGPSV |
| | | | Sequence | FLFPPKPKDTLMISRTPEVTCVVVDVSHEDPEVKFNWYVD |
| | | | | GVEVHNAKTKPREEQYNSTYRVVSVLTVLHQDWLNGKEY |
| | | | | KCKVSNKALPAPIEKTISKAKGQPREPQVYTLPPSRDELTKN |
| | | | | QVSLTCLVKGFYPSDIAVEWESNGQPENNYKTTPPVLDSD |
| | | | | GSFFLYSKLTVDKSRWQQGNIFSCSVMHEALHNHYTQKSL |
| | | | | SLSPGK |
| 95 | Disabled | | Disabled Human | Gcctccaccaagggcccatcggtcttccccctggcaccctcctccaagag |
| | Human | | IGHG1*01 Heavy | cacctctgggggcacagcggccctgggctgcctggtcaaggactacttcc |
| | IgG1 | Disabled | Chain Constant Region | ccgaaccggtgacggtgtcgtggaactcaggcgccctgaccagcggcgtg |
| | heavy | human | Nucleotide Sequence. | cacaccttcccggctgtcctacagtcctcaggactctactccctcagcagc |
| | chain | IGHG1*01 | | gtggtgaccgtgccctccagcagcttgggcacccagacctacatctgcaa |
| | constant | | | cgtgaatcacaagcccagcaacaccaaggtggacaagaaagtggagcc |
| | region | | | caaatcttgtgacaaaactcacacatgcccaccgtgcccagcacctgaac |
| | | | | tcgcgggggcaccgtcagtcttcctcttccccccaaaacccaaggacaccc |
| | | | | tcatgatctcccggacccctgaggtcacatgcgtggtggtggacgtgagcc |
| | | | | acgaagaccctgaggtcaagttcaactggtacgtggacggcgtggaggt |
| | | | | gcataatgccaagacaaagccgcgggaggagcagtacaacagcacgta |
| | | | | ccgtgtggtcagcgtcctcaccgtcctgcaccaggactggctgaatggcaa |
| | | | | ggagtacaagtgcaaggtctccaacaaagccctcccagcccccatcgag |
| | | | | aaaaccatctccaaagccaaagggcagccccgagaaccacaggtgtaca |
| | | | | ccctgcccccatcccgggatgagctgaccaagaaccaggtcagcctgacc |
| | | | | tgcctggtcaaaggcttctatcccagcgacatcgccgtggagtgggagag |
| | | | | caatgggcagccggagaacaactacaagaccacgcctcccgtgctggac |
| | | | | tccgacggctccttcttcctctacagcaagctcaccgtggacaagagcagg |
| | | | | tggcagcaggggaacgtcttctcatgctccgtgatgcatgaggctctgcac |
| | | | | aaccactacacgcagaagagcctctccctgtctccgggtaaa |
| 96 | | | Disabled Human | ASTKGPSVFPLAPSSKSTSGGTAALGCLVKDYFPEPVTVSW |
| | | | IGHG1*01 Heavy | NSGALTSGVHTFPAVLQSSGLYSLSSVVTVPSSSLGTQTYIC |
| | | | Chain Constant Region | NVNHKPSNTKVDKKVEPKSCDKTHTCPPCPAPELAGAPSV |
| | | | Amino Acid Sequence. | FLFPPKPKDTLMISRTPEVTCVVVDVSHEDPEVKFNWYVD |
| | | | Two residues that | GVEVHNAKTKPREEQYNSTYRVVSVLTVLHQDWLNGKEY |
| | | | differ from the wild- | KCKVSNKALPAPIEKTISKAKGQPREPQVYTLPPSRDELTKN |
| | | | type sequence are | QVSLTCLVKGFYPSDIAVEWESNGQPENNYKTTPPVLDSD |
| | | | identified in bold. | GSFFLYSKLTVDKSRWQQGNVFSCSVMHEALHNHYTQKS |
| | | | | LSLSPGK |
| 97 | Human | IGHG2*01 | Human Heavy Chain | gcctccaccaagggcccatcggtcttccccctggcgccctgctccaggagc |
| | IgG2 | or | Constant Region | acctccgagagcacagccgccctgggctgcctggtcaaggactacttccc |
| | constant | IGHG2*04 | (IGHG2*01 or | cgaaccggtgacggtgtcgtggaactcaggcgctctgaccagcggcgtgc |
| | region | or | IGHG2*03 or | acaccttcccagctgtcctacagtcctcaggactctactccctcagcagcgt |
| | | IGHG2*05 | IGHG2*05) Nucleotide | ggtgaccgtgccctccagcaacttcggcacccagacctacacctgcaacg |
| | | | Sequence | tagatcacaagcccagcaacaccaaggtggacaagacagttgagcgcaa |
| | | | | atgttgtgtcgagtgcccaccgtgcccagcaccacctgtggcaggaccgtc |
| | | | | agtcttcctcttccccccaaaacccaaggacaccctcatgatctcccggac |
| | | | | ccctgaggtcacgtgcgtggtggtggacgtgagccacgaagaccccgag |
| | | | | gtccagttcaactggtacgtggacggcgtggaggtgcataatgccaagac |
| | | | | aaagccacgggaggagcagttcaacagcacgttccgtgtggtcagcgtcc |
| | | | | tcaccgttgtgcaccaggactggctgaacggcaaggagtacaagtgcaa |
| | | | | ggtctccaacaaaggcctcccagcccccatcgagaaaaccatctccaaaa |
| | | | | ccaaagggcagccccgagaaccacaggtgtacaccctgcccccatcccg |
| | | | | ggaggagatgaccaagaaccaggtcagcctgacctgcctggtcaaaggc |
| | | | | ttctaccccagcgacatcgccgtggagtgggagagcaatgggcagccgg |
| | | | | agaacaactacaagaccacacctcccatgctggactccgacggctccttc |
| | | | | ttcctctacagcaagctcaccgtggacaagagcaggtggcagcagggga |
| | | | | acgtcttctcatgctccgtgatgcatgaggctctgcacaaccactacacgc |
| | | | | agaagagcctctccctgtctccgggtaaa |
| 98 | | | Human Heavy Chain | ASTKGPSVFPLAPCSRSTSESTAALGCLVKDYFPEPVTVSW |
| | | | Constant Region | NSGALTSGVHTFPAVLQSSGLYSLSSVVTVPSSNFGTQTYT |
| | | | (IGHG2*01) Protein | CNVDHKPSNTKVDKTVERKCCVECPPCPAPPVAGPSVFLF |
| | | | Sequence | PPKPKDTLMISRTPEVTCVVVDVSHEDPEVQFNWYVDGV |
| | | | | EVHNAKTKPREEQFNSTFRVVSVLTVVHQDWLNGKEYKC |
| | | | | KVSNKGLPAPIEKTISKTKGQPREPQVYTLPPSREEMTKNQ |
| | | | | VSLTCLVKGFYPSDIAVEWESNGQPENNYKTTPPMLDSDG |
| | | | | SFFLYSKLTVDKSRWQQGNVFSCSVMHEALHNHYTQKSLS |
| | | | | LSPGK |
| 99 | Human | IGHG2*02 | Human Heavy Chain | GCCTCCACCAAGGGCCCATCGGTCTTCCCCCTGGCGCCC |
| | IgG2 | | Constant Region | TGCTCCAGGAGCACCTCCGAGAGCACAGCGGCCCTGGG |
| | constant | | (IGHG2*02) | CTGCCTGGTCAAGGACTACTTCCCCGAACCGGTGACGGT |
| | region | | Nucleotide Sequence | GTCGTGGAACTCAGGCGCTCTGACCAGCGGCGTGCACA |
| | | | | CCTTCCCGGCTGTCCTACAGTCCTCAGGACTCTACTCCCT |
| | | | | CAGCAGCGTGGTGACCGTGACCTCCAGCAACTTCGGCA |
| | | | | CCCAGACCTACACCTGCAACGTAGATCACAAGCCCAGCA |
| | | | | ACACCAAGGTGGACAAGACAGTTGAGCGCAAATGTTGT |
| | | | | GTCGAGTGCCCACCGTGCCCAGCACCACCTGTGGCAGG |
| | | | | ACCGTCAGTCTTCCTCTTCCCCCCAAAACCCAAGGACACC |
| | | | | CTCATGATCTCCCGGACCCCTGAGGTCACGTGCGTGGTG |
| | | | | GTGGACGTGAGCCACGAAGACCCCGAGGTCCAGTTCAA |
| | | | | CTGGTACGTGGACGGCATGGAGGTGCATAATGCCAAGA |
| | | | | CAAAGCCACGGGAGGAGCAGTTCAACAGCACGTTCCGT |
| | | | | GTGGTCAGCGTCCTCACCGTCGTGCACCAGGACTGGCTG |
| | | | | AACGGCAAGGAGTACAAGTGCAAGGTCTCCAACAAAGG |
| | | | | CCTCCCAGCCCCCATCGAGAAAACCATCTCCAAAACCAA |
| | | | | AGGGCAGCCCCGAGAACCACAGGTGTACACCCTGCCCC |
| | | | | CATCCCGGGAGGAGATGACCAAGAACCAGGTCAGCCTG |
| | | | | ACCTGCCTGGTCAAAGGCTTCTACCCCAGCGACATCGCC |
| | | | | GTGGAGTGGGAGAGCAATGGGCAGCCGGAGAACAACT |
| | | | | ACAAGACCACACCTCCCATGCTGGACTCCGACGGCTCCT |
| | | | | TCTTCCTCTACAGCAAGCTCACCGTGGACAAGAGCAGGT |
| | | | | GGCAGCAGGGGAACGTCTTCTCATGCTCCGTGATGCAT |
| | | | | GAGGCTCTGCACAACCACTACACACAGAAGAGCCTCTCC |
| | | | | CTGTCTCCGGGTAAA |
| 100 | | | Human Heavy Chain | ASTKGPSVFPLAPCSRSTSESTAALGCLVKDYFPEPVTVSW |
| | | | Constant Region | NSGALTSGVHTFPAVLQSSGLYSLSSVVTVTSSNFGTQTYT |
| | | | (IGHG2*02) Protein | CNVDHKPSNTKVDKTVERKCCVECPPCPAPPVAGPSVFLF |
| | | | Sequence | PPKPKDTLMISRTPEVTCVVVDVSHEDPEVQFNWYVDGM |
| | | | | EVHNAKTKPREEQFNSTFRVVSVLTVVHQDWLNGKEYKC |
| | | | | KVSNKGLPAPIEKTISKTKGQPREPQVYTLPPSREEMTKNQ |
| | | | | VSLTCLVKGFYPSDIAVEWESNGQPENNYKTTPPMLDSDG |
| | | | | SFFLYSKLTVDKSRWQQGNVFSCSVMHEALHNHYTQKSLS |
| | | | | LSPGK |
| 101 | Human | IGHG2*04 | Human Heavy Chain | gcctccaccaagggcccatcggtcttccccctggcgccctgctccaggagc |
| | IgG2 | | Constant Region | acctccgagagcacagcggccctgggctgcctggtcaaggactacttccc |
| | constant | | (IGHG2*04) | cgaaccggtgacggtgtcgtggaactcaggcgctctgaccagcggcgtgc |
| | region | | Nucleotide Sequence | acaccttcccagctgtcctacagtcctcaggactctactccctcagcagcgt |
| | | | | ggtgaccgtgccctccagcagcttgggcacccagacctacacctgcaacg |
| | | | | tagatcacaagcccagcaacaccaaggtggacaagacagttgagcgcaa |
| | | | | atgttgtgtcgagtgcccaccgtgcccagcaccacctgtggcaggaccgtc |
| | | | | agtcttcctcttccccccaaaacccaaggacaccctcatgatctcccggac |
| | | | | ccctgaggtcacgtgcgtggtggtggacgtgagccacgaagaccccgag |
| | | | | gtccagttcaactggtacgtggacggcgtggaggtgcataatgccaagac |
| | | | | aaagccacgggaggagcagttcaacagcacgttccgtgtggtcagcgtcc |
| | | | | tcaccgttgtgcaccaggactggctgaacggcaaggagtacaagtgcaa |
| | | | | ggtctccaacaaaggcctcccagcccccatcgagaaaaccatctccaaaa |
| | | | | ccaaagggcagccccgagaaccacaggtgtacaccctgcccccatcccg |
| | | | | ggaggagatgaccaagaaccaggtcagcctgacctgcctggtcaaaggc |
| | | | | ttctaccccagcgacatcgccgtggagtgggagagcaatgggcagccgg |
| | | | | agaacaactacaagaccacacctcccatgctggactccgacggctccttc |
| | | | | ttcctctacagcaagctcaccgtggacaagagcaggtggcagcagggga |
| | | | | acgtcttctcatgctccgtgatgcatgaggctctgcacaaccactacacgc |
| | | | | agaagagcctctccctgtctccgggtaaa |
| 102 | | | Human Heavy Chain | ASTKGPSVFPLAPCSRSTSESTAALGCLVKDYFPEPVTVSW |
| | | | Constant Region | NSGALTSGVHTFPAVLQSSGLYSLSSVVTVPSSSLGTQTYTC |
| | | | (IGHG2*04) Protein | NVDHKPSNTKVDKTVERKCCVECPPCPAPPVAGPSVFLFP |
| | | | Sequence | PKPKDTLMISRTPEVTCVVVDVSHEDPEVQFNWYVDGVE |
| | | | | VHNAKTKPREEQFNSTFRVVSVLTVVHQDWLNGKEYKCK |
| | | | | VSNKGLPAPIEKTISKTKGQPREPQVYTLPPSREEMTKNQV |
| | | | | SLTCLVKGFYPSDIAVEWESNGQPENNYKTTPPMLDSDGS |
| | | | | FFLYSKLTVDKSRWQQGNVFSCSVMHEALHNHYTQKSLSL |
| | | | | SPGK |
| 103 | Human | IGHG2*06 | Human Heavy Chain | GCCTCCACCAAGGGCCCATCGGTCTTCCCCCTGGCGCCC |
| | IgG2 | | Constant Region | TGCTCCAGGAGCACCTCCGAGAGCACAGCGGCCCTGGG |
| | constant | | (IGHG2*06) | CTGCCTGGTCAAGGACTACTTCCCCGAACCGGTGACGGT |
| | region | | Nucleotide Sequence | GTCGTGGAACTCAGGCGCTCTGACCAGCGGCGTGCACA |
| | | | | CCTTCCCGGCTGTCCTACAGTCCTCAGGACTCTACTCCCT |
| | | | | CAGCAGCGTGGTGACCGTGCCCTCCAGCAACTTCGGCAC |
| | | | | CCAGACCTACACCTGCAACGTAGATCACAAGCCCAGCAA |
| | | | | CACCAAGGTGGACAAGACAGTTGAGCGCAAATGTTGTG |
| | | | | TCGAGTGCCCACCGTGCCCAGCACCACCTGTGGCAGGA |
| | | | | CCGTCAGTCTTCCTCTTCCCCCCAAAACCCAAGGACACCC |
| | | | | TCATGATCTCCCGGACCCCTGAGGTCACGTGCGTGGTGG |
| | | | | TGGACGTGAGCCACGAAGACCCCGAGGTCCAGTTCAAC |
| | | | | TGGTACGTGGACGGCGTGGAGGTGCATAATGCCAAGAC |
| | | | | AAAGCCACGGGAGGAGCAGTTCAACAGCACGTTCCGTG |
| | | | | TGGTCAGCGTCCTCACCGTCGTGCACCAGGACTGGCTGA |
| | | | | ACGGCAAGGAGTACAAGTGCAAGGTCTCCAACAAAGGC |
| | | | | CTCCCAGCCCCCATCGAGAAAACCATCTCCAAAACCAAA |
| | | | | GGGCAGCCCCGAGAACCACAGGTGTACACCCTGCCCCC |
| | | | | ATCCCGGGAGGAGATGACCAAGAACCAGGTCAGCCTGA |
| | | | | CCTGCCTGGTCAAAGGCTTCTACCCCAGCGACATCTCCG |
| | | | | TGGAGTGGGAGAGCAATGGGCAGCCGGAGAACAACTA |
| | | | | CAAGACCACACCTCCCATGCTGGACTCCGACGGCTCCTT |
| | | | | CTTCCTCTACAGCAAGCTCACCGTGGACAAGAGCAGGTG |
| | | | | GCAGCAGGGGAACGTCTTCTCATGCTCCGTGATGCATGA |
| | | | | GGCTCTGCACAACCACTACACACAGAAGAGCCTCTCCCT |
| | | | | GTCTCCGGGTAAA |
| 104 | | | Human Heavy Chain | ASTKGPSVFPLAPCSRSTSESTAALGCLVKDYFPEPVTVSW |
| | | | Constant Region | NSGALTSGVHTFPAVLQSSGLYSLSSVVTVPSSNFGTQTYT |
| | | | (IGHG2*06) Protein | CNVDHKPSNTKVDKTVERKCCVECPPCPAPPVAGPSVFLF |
| | | | Sequence | PPKPKDTLMISRTPEVTCVVVDVSHEDPEVQFNWYVDGV |
| | | | | EVHNAKTKPREEQFNSTFRVVSVLTVVHQDWLNGKEYKC |
| | | | | KVSNKGLPAPIEKTISKTKGQPREPQVYTLPPSREEMTKNQ |
| | | | | VSLTCLVKGFYPSDISVEWESNGQPENNYKTTPPMLDSDG |
| | | | | SFFLYSKLTVDKSRWQQGNVFSCSVMHEALHNHYTQKSLS |
| | | | | LSPGK |
| 105 | Human | IGHG4*01 | Human Heavy Chain | gcttccaccaagggcccatccgtcttccccctggcgccctgctccaggagc |
| | IgG4 | or | Constant Region | acctccgagagcacagccgccctgggctgcctggtcaaggactacttccc |
| | constant | IGHG4*04 | (IGHG4*01 or | cgaaccggtgacggtgtcgtggaactcaggcgccctgaccagcggcgtgc |
| | region | | IGHG4*04) Nucleotide | acaccttcccggctgtcctacagtcctcaggactctactccctcagcagcgt |
| | | | Sequence | ggtgaccgtgccctccagcagcttgggcacgaagacctacacctgcaacg |
| | | | | tagatcacaagcccagcaacaccaaggtggacaagagagttgagtccaa |
| | | | | atatggtcccccatgcccatcatgcccagcacctgagttcctggggggacc |
| | | | | atcagtcttcctgttccccccaaaacccaaggacactctcatgatctcccg |
| | | | | gacccctgaggtcacgtgcgtggtggtggacgtgagccaggaagacccc |
| | | | | gaggtccagttcaactggtacgtggatggcgtggaggtgcataatgccaa |
| | | | | gacaaagccgcgggaggagcagttcaacagcacgtaccgtgtggtcagc |
| | | | | gtcctcaccgtcctgcaccaggactggctgaacggcaaggagtacaagtg |
| | | | | caaggtctccaacaaaggcctcccgtcctccatcgagaaaaccatctcca |
| | | | | aagccaaagggcagccccgagagccacaggtgtacaccctgcccccatc |
| | | | | ccaggaggagatgaccaagaaccaggtcagcctgacctgcctggtcaaa |
| | | | | ggcttctaccccagcgacatcgccgtggagtgggagagcaatgggcagc |
| | | | | cggagaacaactacaagaccacgcctcccgtgctggactccgacggctcc |
| | | | | ttcttcctctacagcaggctaaccgtggacaagagcaggtggcaggaggg |
| | | | | gaatgtcttctcatgctccgtgatgcatgaggctctgcacaaccactacac |
| | | | | acagaagagcctctccctgtctctgggtaaa |
| 106 | | | Human Heavy Chain | ASTKGPSVFPLAPCSRSTSESTAALGCLVKDYFPEPVTVSW |
| | | | Constant Region | NSGALTSGVHTFPAVLQSSGLYSLSSVVTVPSSSLGTKTYTC |
| | | | (IGHG4*01) Protein | NVDHKPSNTKVDKRVESKYGPPCPSCPAPEFLGGPSVFLFP |
| | | | Sequence (P01861) | PKPKDTLMISRTPEVTCVVVDVSQEDPEVQFNWYVDGVE |
| | | | | VHNAKTKPREEQFNSTYRVVSVLTVLHQDWLNGKEYKCKV |
| | | | | SNKGLPSSIEKTISKAKGQPREPQVYTLPPSQEEMTKNQVS |
| | | | | LTCLVKGFYPSDIAVEWESNGQPENNYKTTPPVLDSDGSFF |
| | | | | LYSRLTVDKSRWQEGNVFSCSVMHEALHNHYTQKSLSLSL |
| | | | | GK |
| 107 | Human | IGHG4*02 | Human Heavy Chain | gcttccaccaagggcccatccgtcttccccctggcgccctgctccaggagc |
| | IgG4 | | Constant Region | acctccgagagcacagccgccctgggctgcctggtcaaggactacttccc |
| | constant | | (IGHG4*02) | cgaaccggtgacggtgtcgtggaactcaggcgccctgaccagcggcgtgc |
| | region | | Nucleotide Sequence | acaccttcccggctgtcctacagtcctcaggactctactccctcagcagcgt |
| | | | | ggtgaccgtgccctccagcagcttgggcacgaagacctacacctgcaacg |
| | | | | tagatcacaagcccagcaacaccaaggtggacaagagagttgagtccaa |
| | | | | atatggtcccccgtgcccatcatgcccagcacctgagttcctggggggacc |
| | | | | atcagtcttcctgttccccccaaaacccaaggacactctcatgatctcccg |
| | | | | gacccctgaggtcacgtgcgtggtggtggacgtgagccaggaagacccc |
| | | | | gaggtccagttcaactggtacgtggatggcgtggaggtgcataatgccaa |
| | | | | gacaaagccgcgggaggagcagttcaacagcacgtaccgtgtggtcagc |
| | | | | gtcctcaccgtcgtgcaccaggactggctgaacggcaaggagtacaagtg |
| | | | | caaggtctccaacaaaggcctcccgtcctccatcgagaaaaccatctcca |
| | | | | aagccaaagggcagccccgagagccacaggtgtacaccctgcccccatc |
| | | | | ccaggaggagatgaccaagaaccaggtcagcctgacctgcctggtcaaa |
| | | | | ggcttctaccccagcgacatcgccgtggagtgggagagcaatgggcagc |
| | | | | cggagaacaactacaagaccacgcctcccgtgctggactccgacggctcc |
| | | | | ttcttcctctacagcaggctaaccgtggacaagagcaggtggcaggaggg |
| | | | | gaatgtcttctcatgctccgtgatgcatgaggctctgcacaaccactacac |
| | | | | gcagaagagcctctccctgtctctgggtaaa |
| 108 | | | Human Heavy Chain | ASTKGPSVFPLAPCSRSTSESTAALGCLVKDYFPEPVTVSW |
| | | | Constant Region | NSGALTSGVHTFPAVLQSSGLYSLSSVVTVPSSSLGTKTYTC |
| | | | (IGHG4*02) Protein | NVDHKPSNTKVDKRVESKYGPPCPSCPAPEFLGGPSVFLFP |
| | | | Sequence | PKPKDTLMISRTPEVTCVVVDVSQEDPEVQFNWYVDGVE |
| | | | | VHNAKTKPREEQFNSTYRVVSVLTVVHQDWLNGKEYKCK |
| | | | | VSNKGLPSSIEKTISKAKGQPREPQVYTLPPSQEEMTKNQV |
| | | | | SLTCLVKGFYPSDIAVEWESNGQPENNYKTTPPVLDSDGSF |
| | | | | FLYSRLTVDKSRWQEGNVFSCSVMHEALHNHYTQKSLSLS |
| | | | | LGK |
| 109 | Human | IGHG4*03 | Human Heavy Chain | gcttccaccaagggcccatccgtcttccccctggcgccctgctccaggagc |
| | IgG4 | | Constant Region | acctccgagagcacagccgccctgggctgcctggtcaaggactacttccc |
| | constant | | (IGHG4*03) | cgaaccggtgacggtgtcgtggaactcaggcgccctgaccagcggcgtgc |
| | region | | Nucleotide Sequence | acaccttcccggctgtcctacagtcctcaggactctactccctcagcagcgt |
| | | | | ggtgaccgtgccctccagcagcttgggcacgaagacctacacctgcaacg |
| | | | | tagatcacaagcccagcaacaccaaggtggacaagagagttgagtccaa |
| | | | | atatggtcccccatgcccatcatgcccagcacctgagttcctggggggacc |
| | | | | atcagtcttcctgttccccccaaaacccaaggacactctcatgatctcccg |
| | | | | gacccctgaggtcacgtgcgtggtggtggacgtgagccaggaagacccc |
| | | | | gaggtccagttcaactggtacgtggatggcgtggaggtgcataatgccaa |
| | | | | gacaaagccgcgggaggagcagttcaacagcacgtaccgtgtggtcagc |
| | | | | gtcctcaccgtcctgcaccaggactggctgaacggcaaggagtacaagtg |
| | | | | caaggtctccaacaaaggcctcccgtcctccatcgagaaaaccatctcca |
| | | | | aagccaaagggcagccccgagagccacaggtgtacaccctgcccccatc |
| | | | | ccaggaggagatgaccaagaaccaggtcagcctgacctgcctggtcaaa |
| | | | | ggcttctaccccagcgacatcgccgtggagtgggagagcaatgggcagc |
| | | | | cggagaacaactacaagaccacgcctcccgtgctggactccgacggctcc |
| | | | | ttcttcctctacagcaagctcaccgtggacaagagcaggtggcaggaggg |
| | | | | gaacgtcttctcatgctccgtgatgcatgaggctctgcacaaccactacac |
| | | | | gcagaagagcctctccctgtctctgggtaaa |
| 110 | | | Human Heavy Chain | ASTKGPSVFPLAPCSRSTSESTAALGCLVKDYFPEPVTVSW |
| | | | Constant Region | NSGALTSGVHTFPAVLQSSGLYSLSSVVTVPSSSLGTKTYTC |
| | | | (IGHG4*03) Protein | NVDHKPSNTKVDKRVESKYGPPCPSCPAPEFLGGPSVFLFP |
| | | | Sequence | PKPKDTLMISRTPEVTCVVVDVSQEDPEVQFNWYVDGVE |
| | | | | VHNAKTKPREEQFNSTYRVVSVLTVLHQDWLNGKEYKCKV |
| | | | | SNKGLPSSIEKTISKAKGQPREPQVYTLPPSQEEMTKNQVS |
| | | | | LTCLVKGFYPSDIAVEWESNGQPENNYKTTPPVLDSDGSFF |
| | | | | LYSKLTVDKSRWQEGNVFSCSVMHEALHNHYTQKSLSLSL |
| | | | | GK |
| 111 | Human | IGHG4-PE | Human Heavy Chain | gcctccaccaagggcccatccgtcttccccctggcgccctgctccaggagc |
| | IgG4- | | Constant Region | acctccgagagcacggccgccctgggctgcctggtcaaggactacttccc |
| | PE | | (IGHG4-PE) Nucleotide | cgaaccagtgacggtgtcgtggaactcaggcgccctgaccagcggcgtgc |
| | constant | | Sequence Version A | acaccttcccggctgtcctacagtcctcaggactctactccctcagcagcgt |
| | region | | | ggtgaccgtgccctccagcagcttgggcacgaagacctacacctgcaacg |
| | | | | tagatcacaagcccagcaacaccaaggtggacaagagagttgagtccaa |
| | | | | atatggtcccccatgcccaccatgcccagcgcctgaatttgaggggggac |
| | | | | catcagtcttcctgttccccccaaaacccaaggacactctcatgatctcccg |
| | | | | gacccctgaggtcacgtgcgtggtggtggacgtgagccaggaagacccc |
| | | | | gaggtccagttcaactggtacgtggatggcgtggaggtgcataatgccaa |
| | | | | gacaaagccgcgggaggagcagttcaacagcacgtaccgtgtggtcagc |
| | | | | gtcctcaccgtcctgcaccaggactggctgaacggcaaggagtacaagtg |
| | | | | caaggtctccaacaaaggcctcccgtcatcgatcgagaaaaccatctcca |
| | | | | aagccaaagggcagccccgagagccacaggtgtacaccctgcccccatc |
| | | | | ccaggaggagatgaccaagaaccaggtcagcctgacctgcctggtcaaa |
| | | | | ggcttctaccccagcgacatcgccgtggagtgggagagcaatgggcagc |
| | | | | cggagaacaactacaagaccacgcctcccgtgctggactccgacggatcc |
| | | | | ttcttcctctacagcaggctaaccgtggacaagagcaggtggcaggaggg |
| | | | | gaatgtcttctcatgctccgtgatgcatgaggctctgcacaaccactacac |
| | | | | acagaagagcctctccctgtctctgggtaaa |
| 112 | | | Human Heavy Chain | gcctccaccaagggacctagcgtgttccctctcgccccctgttccaggtcc |
| | | | Constant Region | acaagcgagtccaccgctgccctcggctgtctggtgaaagactactttccc |
| | | | (IGHG4-PE) Nucleotide | gagcccgtgaccgtctcctggaatagcggagccctgacctccggcgtgca |
| | | | Sequence Version B | cacatttcccgccgtgctgcagagcagcggactgtatagcctgagcagcg |
| | | | | tggtgaccgtgcccagctccagcctcggcaccaaaacctacacctgcaac |
| | | | | gtggaccacaagccctccaacaccaaggtggacaagcgggtggagagc |
| | | | | aagtacggccccccttgccctccttgtcctgcccctgagttcgagggagga |
| | | | | ccctccgtgttcctgtttccccccaaacccaaggacaccctgatgatctccc |
| | | | | ggacacccgaggtgacctgtgtggtcgtggacgtcagccaggaggaccc |
| | | | | cgaggtgcagttcaactggtatgtggacggcgtggaggtgcacaatgcca |
| | | | | aaaccaagcccagggaggagcagttcaattccacctacagggtggtgag |
| | | | | cgtgctgaccgtcctgcatcaggattggctgaacggcaaggagtacaagt |
| | | | | gcaaggtgtccaacaagggactgcccagctccatcgagaagaccatcag |
| | | | | caaggctaagggccagccgagggagccccaggtgtataccctgcctccta |
| | | | | gccaggaagagatgaccaagaaccaagtgtccctgacctgcctggtgaa |
| | | | | gggattctacccctccgacatcgccgtggagtgggagagcaatggccagc |
| | | | | ccgagaacaactacaaaacaacccctcccgtgctcgatagcgacggcag |
| | | | | cttctttctctacagccggctgacagtggacaagagcaggtggcaggagg |
| | | | | gcaacgtgttctcctgttccgtgatgcacgaggccctgcacaatcactaca |
| | | | | cccagaagagcctctccctgtccctgggcaag |
| 113 | | | Human Heavy Chain | gccagcaccaagggcccttccgtgttccccctggccccttgcagcaggagc |
| | | | Constant Region | acctccgaatccacagctgccctgggctgtctggtgaaggactactttccc |
| | | | (IGHG4-PE) Nucleotide | gagcccgtgaccgtgagctggaacagcggcgctctgacatccggcgtcca |
| | | | Sequence Version C | cacctttcctgccgtcctgcagtcctccggcctctactccctgtcctccgtgg |
| | | | | tgaccgtgcctagctcctccctcggcaccaagacctacacctgtaacgtgg |
| | | | | accacaaaccctccaacaccaaggtggacaaacgggtcgagagcaagt |
| | | | | acggccctccctgccctccttgtcctgcccccgagttcgaaggcggaccca |
| | | | | gcgtgttcctgttccctcctaagcccaaggacaccctcatgatcagccgga |
| | | | | cacccgaggtgacctgcgtggtggtggatgtgagccaggaggaccctga |
| | | | | ggtccagttcaactggtatgtggatggcgtggaggtgcacaacgccaaga |
| | | | | caaagccccgggaagagcagttcaactccacctacagggtggtcagcgt |
| | | | | gctgaccgtgctgcatcaggactggctgaacggcaaggagtacaagtgc |
| | | | | aaggtcagcaataagggactgcccagcagcatcgagaagaccatctcca |
| | | | | aggctaaaggccagccccgggaacctcaggtgtacaccctgcctcccagc |
| | | | | caggaggagatgaccaagaaccaggtgagcctgacctgcctggtgaagg |
| | | | | gattctacccttccgacatcgccgtggagtgggagtccaacggccagccc |
| | | | | gagaacaattataagaccacccctcccgtcctcgacagcgacggatcctt |
| | | | | ctttctgtactccaggctgaccgtggataagtccaggtggcaggaaggca |
| | | | | acgtgttcagctgctccgtgatgcacgaggccctgcacaatcactacaccc |
| | | | | agaagtccctgagcctgtccctgggaaag |
| 114 | | | Human Heavy Chain | ASTKGPSVFPLAPCSRSTSESTAALGCLVKDYFPEPVTVSW |
| | | | Constant Region | NSGALTSGVHTFPAVLQSSGLYSLSSVVTVPSSSLGTKTYTC |
| | | | (IGHG4-PE) Protein | NVDHKPSNTKVDKRVESKYGPPCPPCPAPEFEGGPSVFLFP |
| | | | Sequence (Amino acid | PKPKDTLMISRTPEVTCVVVDVSQEDPEVQFNWYVDGVE |
| | | | substitution shown in | VHNAKTKPREEQFNSTYRVVSVLTVLHQDWLNGKEYKCKV |
| | | | BOLD) | SNKGLPSSIEKTISKAKGQPREPQVYTLPPSQEEMTKNQVS |
| | | | | LTCLVKGFYPSDIAVEWESNGQPENNYKTTPPVLDSDGSFF |
| | | | | LYSRLTVDKSRWQEGNVFSCSVMHEALHNHYTQKSLSLSL |
| | | | | GK |
| 115 | Inactivated | Inacti- | Inactivated Human | gcctccaccaagggcccatccgtcttccccctggcgccctgctccaggagc |
| | Human | vated | Heavy Chain Constant | acctccgagagcacggccgccctgggctgcctggtcaaggactacttccc |
| | IgG4 | | Region (IGHG4) | cgaaccagtgacggtgtcgtggaactcaggcgccctgaccagcggcgtgc |
| | constant | | Nucleotide Sequence | acaccttcccggctgtcctacagtcctcaggactctactccctcagcagcgt |
| | region | | | ggtgaccgtgccctccagcagcttgggcacgaagacctacacctgcaacg |
| | | | | tagatcacaagcccagcaacaccaaggtggacaagagagttgagtccaa |
| | | | | atatggtcccccatgcccaccatgcccagcgcctccagttgcggggggac |
| | | | | catcagtcttcctgttccccccaaaacccaaggacactctcatgatctcccg |
| | | | | gacccctgaggtcacgtgcgtggtggtggacgtgagccaggaagacccc |
| | | | | gaggtccagttcaactggtacgtggatggcgtggaggtgcataatgccaa |
| | | | | gacaaagccgcgggaggagcagttcaacagcacgtaccgtgtggtcagc |
| | | | | gtcctcaccgtcctgcaccaggactggctgaacggcaaggagtacaagtg |
| | | | | caaggtctccaacaaaggcctcccgtcatcgatcgagaaaaccatctcca |
| | | | | aagccaaagggcagccccgagagccacaggtgtacaccctgcccccatc |
| | | | | ccaggaggagatgaccaagaaccaggtcagcctgacctgcctggtcaaa |
| | | | | ggcttctaccccagcgacatcgccgtggagtgggagagcaatgggcagc |
| | | | | cggagaacaactacaagaccacgcctcccgtgctggactccgacggatcc |
| | | | | ttcttcctctacagcaggctaaccgtggacaagagcaggtggcaggaggg |
| | | | | gaatgtcttctcatgctccgtgatgcatgaggctctgcacaaccactacac |
| | | | | acagaagagcctctccctgtctctgggtaaa |
| 116 | | IGHG4 | Inactivated Human | ASTKGPSVFPLAPCSRSTSESTAALGCLVKDYFPEPVTVSW |
| | | | Heavy Chain Constant | NSGALTSGVHTFPAVLQSSGLYSLSSVVTVPSSSLGTKTYTC |
| | | | Region (IGHG4) | NVDHKPSNTKVDKRVESKYGPPCPPCPAPPVAGGPSVFLF |
| | | | Protein Sequence | PPKPKDTLMISRTPEVTCVVVDVSQEDPEVQFNWYVDGV |
| | | | (inactivating | EVHNAKTKPREEQFNSTYRVVSVLTVLHQDWLNGKEYKCK |
| | | | mutations from | VSNKGLPSSIEKTISKAKGQPREPQVYTLPPSQEEMTKNQV |
| | | | human IgG4 shown in | SLTCLVKGFYPSDIAVEWESNGQPENNYKTTPPVLDSDGSF |
| | | | bold) | FLYSRLTVDKSRWQEGNVFSCSVMHEALHNHYTQKSLSLS |
| | | | | LGK |
| 117 | Human | IGKC*01 | Human Cκ Light Chain | cgtacggtggccgctccctccgtgttcatcttcccaccttccgacgagcagc |
| | Cκ | | Constant Region | tgaagtccggcaccgcttctgtcgtgtgcctgctgaacaacttctacccccg |
| | constant | | (IGKC*01) Nucleotide | cgaggccaaggtgcagtggaaggtggacaacgccctgcagtccggcaac |
| | region | | Sequence | tcccaggaatccgtgaccgagcaggactccaaggacagcacctactccct |
| | | | | gtcctccaccctgaccctgtccaaggccgactacgagaagcacaaggtgt |
| | | | | acgcctgcgaagtgacccaccagggcctgtctagccccgtgaccaagtct |
| | | | | ttcaaccggggcgagtgt |
| 118 | | | Cκ Light Chain | RTVAAPSVFIFPPSDEQLKSGTASVVCLLNNFYPREAKVQW |
| | | | Constant Region | KVDNALQSGNSQESVTEQDSKDSTYSLSSTLTLSKADYEKH |
| | | | (IGKC*01) Amino Acid | KVYACEVTHQGLSSPVTKSFNRGEC |
| | | | Sequence | |
| 119 | Human | IGKC*02 | Cκ Light Chain | cgaactgtggctgcaccatctgtcttcatcttcccgccatctgatgagcagt |
| | Cκ | | Constant Region | tgaaatctggaactgcctctgttgtgtgcctgctgaataacttctatcccag |
| | constant | | (IGKC*02) Nucleotide | agaggccaaagtacagtggaaggtggataacgccctccaatcgggtaac |
| | region | | Sequence | tcccaggagagtgtcacagagcaggagagcaaggacagcacctacagc |
| | | | | ctcagcagcaccctgacgctgagcaaagcagactacgagaaacacaaa |
| | | | | gtctacgccggcgaagtcacccatcagggcctgagctcgcccgtcacaaa |
| | | | | gagcttcaacaggggagagtgt |
| 120 | | | Cκ Light Chain | RTVAAPSVFIFPPSDEQLKSGTASVVCLLNNFYPREAKVQW |
| | | | Constant Region | KVDNALQSGNSQESVTEQESKDSTYSLSSTLTLSKADYEKH |
| | | | (IGKC*02) Amino Acid | KVYAGEVTHQGLSSPVTKSFNRGEC |
| | | | Sequence | |
| 121 | Human | IGKC*03 | Cκ Light Chain | cgaactgtggctgcaccatctgtcttcatcttcccgccatctgatgagcagt |
| | Cκ | | Constant Region | tgaaatctggaactgcctctgttgtgtgcctgctgaataacttctatcccag |
| | constant | | (IGKC*03) Nucleotide | agaggccaaagtacagcggaaggtggataacgccctccaatcgggtaac |
| | region | | Sequence | tcccaggagagtgtcacagagcaggagagcaaggacagcacctacagc |
| | | | | ctcagcagcaccctgacgctgagcaaagcagactacgagaaacacaaa |
| | | | | gtctacgcctgcgaagtcacccatcagggcctgagctcgcccgtcacaaa |
| | | | | gagcttcaacaggggagagtgt |
| 122 | | | Cκ Light Chain | RTVAAPSVFIFPPSDEQLKSGTASVVCLLNNFYPREAKVQR |
| | | | Constant Region | KVDNALQSGNSQESVTEQESKDSTYSLSSTLTLSKADYEKH |
| | | | (IGKC*03) Amino Acid | KVYACEVTHQGLSSPVTKSFNRGEC |
| | | | Sequence | |
| 123 | Human | IGKC*04 | Cκ Light Chain | cgaactgtggctgcaccatctgtcttcatcttcccgccatctgatgagcagt |
| | Cκ | | Constant Region | tgaaatctggaactgcctctgttgtgtgcctgctgaataacttctatcccag |
| | constant | | (IGKC*04) Nucleotide | agaggccaaagtacagtggaaggtggataacgccctccaatcgggtaac |
| | region | | Sequence | tcccaggagagtgtcacagagcaggacagcaaggacagcacctacagc |
| | | | | ctcagcagcaccctgacgctgagcaaagcagactacgagaaacacaaac |
| | | | | tctacgcctgcgaagtcacccatcagggcctgagctcgcccgtcacaaag |
| | | | | agcttcaacaggggagagtgt |
| 124 | | | Cκ Light Chain | RTVAAPSVFIFPPSDEQLKSGTASVVCLLNNFYPREAKVQW |
| | | | Constant Region | KVDNALQSGNSQESVTEQDSKDSTYSLSSTLTLSKADYEKH |
| | | | (IGKC*04) Amino Acid | KLYACEVTHQGLSSPVTKSFNRGEC |
| | | | Sequence | |
| 125 | Human | IGKC*05 | Cκ Light Chain | cgaactgtggctgcaccatctgtcttcatcttcccgccatctgatgagcagt |
| | Cκ | | Constant Region | tgaaatctggaactgcctctgttgtgtgcctgctgaataacttctatcccag |
| | constant | | (IGKC*05) Nucleotide | agaggccaaagtacagtggaaggtggataacgccctccaatcgggtaac |
| | region | | Sequence | tcccaggagagtgtcacagagcaggacagcaaggacagcacctacagc |
| | | | | ctcagcaacaccctgacgctgagcaaagcagactacgagaaacacaaa |
| | | | | gtctacgcctgcgaagtcacccatcagggcctgagctcgcccgtcacaaa |
| | | | | gagcttcaacaggggagagtgc |
| 126 | | | Cκ Light Chain | RTVAAPSVFIFPPSDEQLKSGTASVVCLLNNFYPREAKVQW |
| | | | Constant Region | KVDNALQSGNSQESVTEQDSKDSTYSLSNTLTLSKADYEKH |
| | | | (IGKC*05) Amino Acid | KVYACEVTHQGLSSPVTKSFNRGEC |
| | | | Sequence | |
| 127 | Human | IGLC1*01 | Cλ Light Chain | cccaaggccaaccccacggtcactctgttcccgccctcctctgaggagctc |
| | Cλ | | Constant Region | caagccaacaaggccacactagtgtgtctgatcagtgacttctacccggg |
| | constant | | (IGLC1*01) Nucleotide | agctgtgacagtggcttggaaggcagatggcagccccgtcaaggcggga |
| | region | | Sequence | gtggagacgaccaaaccctccaaacagagcaacaacaagtacgcggcc |
| | | | (ENST00000390321.2) | agcagctacctgagcctgacgcccgagcagtggaagtcccacagaagct |
| | | | | acagctgccaggtcacgcatgaagggagcaccgtggagaagacagtgg |
| | | | | cccctacagaatgttca |
| 128 | | | Cλ Light Chain | PKANPTVTLFPPSSEELQANKATLVCLISDFYPGAVTVAWK |
| | | | Constant Region | ADGSPVKAGVETTKPSKQSNNKYAASSYLSLTPEQWKSHR |
| | | | (IGLC1*01) Amino | SYSCQVTHEGSTVEKTVAPTECS |
| | | | Acid Sequence | |
| | | | (A0A075B6K8) | |
| 129 | Human | IGLC1*02 | Cλ Light Chain | ggtcagcccaaggccaaccccactgtcactctgttcccgccctcctctgag |
| | Cλ | | Constant Region | gagctccaagccaacaaggccacactagtgtgtctgatcagtgacttctac |
| | constant | | (IGLC1*02) Nucleotide | ccgggagctgtgacagtggcctggaaggcagatggcagccccgtcaagg |
| | region | | Sequence Version A | cgggagtggagaccaccaaaccctccaaacagagcaacaacaagtacg |
| | | | | cggccagcagctacctgagcctgacgcccgagcagtggaagtcccacag |
| | | | | aagctacagctgccaggtcacgcatgaagggagcaccgtggagaagac |
| | | | | agtggcccctacagaatgttca |
| 130 | | | Cλ Light Chain | ggtcagcccaaggccaaccccactgtcactctgttcccgccctcctctgag |
| | | | Constant Region | gagctccaagccaacaaggccacactagtgtgtctgatcagtgacttctac |
| | | | (IGLC1*02) Nucleotide | ccgggagctgtgacagtggcctggaaggcagatggcagccccgtcaagg |
| | | | Sequence Version B | cgggagtggagaccaccaaaccctccaaacagagcaacaacaagtacg |
| | | | | cggccagcagctacctgagcctgacgcccgagcagtggaagtcccacag |
| | | | | aagctacagctgccaggtcacgcatgaagggagcaccgtggagaagac |
| | | | | agtggcccctacagaatgttca |
| 131 | | | Cλ Light Chain | GQPKANPTVTLFPPSSEELQANKATLVCLISDFYPGAVTVA |
| | | | Constant Region | WKADGSPVKAGVETTKPSKQSNNKYAASSYLSLTPEQWKS |
| | | | (IGLC1*02) Amino | HRSYSCQVTHEGSTVEKTVAPTECS |
| | | | Acid Sequence | |
| 132 | Human | IGLC2*01 | Cλ Light Chain | ggccagcctaaggccgctccttctgtgaccctgttccccccatcctccgag |
| | Cλ | | Constant Region | gaactgcaggctaacaaggccaccctcgtgtgcctgatcagcgacttcta |
| | constant | | (IGLC2*01) Nucleotide | ccctggcgccgtgaccgtggcctggaaggctgatagctctcctgtgaagg |
| | region | | Sequence Version A | ccggcgtggaaaccaccaccccttccaagcagtccaacaacaaatacgc |
| | | | | cgcctcctcctacctgtccctgacccctgagcagtggaagtcccaccggtc |
| | | | | ctacagctgccaagtgacccacgagggctccaccgtggaaaagaccgtg |
| | | | | gctcctaccgagtgctcc |
| 133 | | | Cλ Light Chain | ggccagcctaaagctgcccccagcgtcaccctgtttcctccctccagcgag |
| | | | Constant Region | gagctccaggccaacaaggccaccctcgtgtgcctgatctccgacttctat |
| | | | (IGLC2*01) Nucleotide | cccggcgctgtgaccgtggcttggaaagccgactccagccctgtcaaagc |
| | | | Sequence Version B | cggcgtggagaccaccacaccctccaagcagtccaacaacaagtacgcc |
| | | | | gcctccagctatctctccctgacccctgagcagtggaagtcccaccggtcc |
| | | | | tactcctgtcaggtgacccacgagggctccaccgtggaaaagaccgtcgc |
| | | | | ccccaccgagtgctcc |
| 134 | | | Cλ Light Chain | GQPKAAPSVTLFPPSSEELQANKATLVCLISDFYPGAVTVA |
| | | | Constant Region | WKADSSPVKAGVETTTPSKQSNNKYAASSYLSLTPEQWKS |
| | | | (IGLC1*02) Amino | HRSYSCQVTHEGSTVEKTVAPTECS |
| | | | Acid Sequence | |
| 135 | Human | IGLC2*02 | Cλ Light Chain | ggtcagcccaaggctgccccctcggtcactctgttcccgccctcctctgagg |
| | Cλ | or | Constant Region | agcttcaagccaacaaggccacactggtgtgtctcataagtgacttctacc |
| | constant | IGLC2*03 | (IGLC2*02 or | cgggagccgtgacagtggcctggaaggcagatagcagccccgtcaaggc |
| | region | | IGLC2*03) Nucleotide | gggagtggagaccaccacaccctccaaacaaagcaacaacaagtacgc |
| | | | Sequence | ggccagcagctatctgagcctgacgcctgagcagtggaagtcccacaga |
| | | | | agctacagctgccaggtcacgcatgaagggagcaccgtggagaagaca |
| | | | | gtggcccctacagaatgttca |
| 136 | | | Cλ Light Chain | GQPKAAPSVTLFPPSSEELQANKATLVCLISDFYPGAVTVA |
| | | | Constant Region | WKADSSPVKAGVETTTPSKQSNNKYAASSYLSLTPEQWKS |
| | | | (IGLC2*02) Amino | HRSYSCQVTHEGSTVEKTVAPTECS |
| | | | Acid Sequence | |
| 137 | Human | IGLC3*01 | Cλ Light Chain | cccaaggctgccccctcggtcactctgttcccaccctcctctgaggagcttc |
| | Cλ | | Constant Region | aagccaacaaggccacactggtgtgtctcataagtgacttctacccggga |
| | constant | | (IGLC3*01) Nucleotide | gccgtgacagttgcctggaaggcagatagcagccccgtcaaggcggggg |
| | region | | Sequence | tggagaccaccacaccctccaaacaaagcaacaacaagtacgcggcca |
| | | | | gcagctacctgagcctgacgcctgagcagtggaagtcccacaaaagcta |
| | | | | cagctgccaggtcacgcatgaagggagcaccgtggagaagacagttgcc |
| | | | | cctacggaatgttca |
| 138 | | | Cλ Light Chain | PKAAPSVTLFPPSSEELQANKATLVCLISDFYPGAVTVAWK |
| | | | Constant Region | ADSSPVKAGVETTTPSKQSNNKYAASSYLSLTPEQWKSHKS |
| | | | (IGLC3*01) Amino | YSCQVTHEGSTVEKTVAPTECS |
| | | | Acid Sequence | |
| 139 | Human | IGLC3*02 | Cλ Light Chain | ggtcagcccaaggctgccccctcggtcactctgttcccaccctcctctgag |
| | Cλ | | Constant Region | gagcttcaagccaacaaggccacactggtgtgtctcataagtgacttctac |
| | constant | | (IGLC3*02) Nucleotide | ccggggccagtgacagttgcctggaaggcagatagcagccccgtcaagg |
| | region | | Sequence | cgggggtggagaccaccacaccctccaaacaaagcaacaacaagtacg |
| | | | | cggccagcagctacctgagcctgacgcctgagcagtggaagtcccacaa |
| | | | | aagctacagctgccaggtcacgcatgaagggagcaccgtggagaagac |
| | | | | agtggcccctacggaatgttca |
| 140 | | | Cλ Light Chain | GQPKAAPSVTLFPPSSEELQANKATLVCLISDFYPGPVTVA |
| | | | Constant Region | WKADSSPVKAGVETTTPSKQSNNKYAASSYLSLTPEQWKS |
| | | | (IGLC1*02) Amino | HKSYSCQVTHEGSTVEKTVAPTECS |
| | | | Acid Sequence | |
| 141 | Human | IGLC3*03 | Cλ Light Chain | ggtcagcccaaggctgccccctcggtcactctgttcccaccctcctctgag |
| | Cλ | | Constant Region | gagcttcaagccaacaaggccacactggtgtgtctcataagtgacttctac |
| | constant | | (IGLC3*03) Nucleotide | ccgggagccgtgacagtggcctggaaggcagatagcagccccgtcaagg |
| | region | | Sequence | cgggagtggagaccaccacaccctccaaacaaagcaacaacaagtacg |
| | | | | cggccagcagctacctgagcctgacgcctgagcagtggaagtcccacaa |
| | | | | aagctacagctgccaggtcacgcatgaagggagcaccgtggagaagac |
| | | | | agtggcccctacagaatgttca |
| 142 | | | Cλ Light Chain | GQPKAAPSVTLFPPSSEELQANKATLVCLISDFYPGAVTVA |
| | | | Constant Region | WKADSSPVKAGVETTTPSKQSNNKYAASSYLSLTPEQWKS |
| | | | (IGLC3*03) Amino | HKSYSCQVTHEGSTVEKTVAPTECS |
| | | | Acid Sequence | |
| 143 | Human | IGLC3*04 | Cλ Light Chain | ggtcagcccaaggctgccccctcggtcactctgttcccgccctcctctgagg |
| | Cλ | | Constant Region | agcttcaagccaacaaggccacactggtgtgtctcataagtgacttctacc |
| | constant | | (IGLC3*04) Nucleotide | cgggagccgtgacagtggcctggaaggcagatagcagccccgtcaaggc |
| | region | | Sequence | gggagtggagaccaccacaccctccaaacaaagcaacaacaagtacgc |
| | | | | ggccagcagctacctgagcctgacgcctgagcagtggaagtcccacaga |
| | | | | agctacagctgccaggtcacgcatgaagggagcaccgtggagaagaca |
| | | | | gtggcccctacagaatgttca |
| 144 | | | Cλ Light Chain | GQPKAAPSVTLFPPSSEELQANKATLVCLISDFYPGAVTVA |
| | | | Constant Region | WKADSSPVKAGVETTTPSKQSNNKYAASSYLSLTPEQWKS |
| | | | (IGLC3*04) Amino | HRSYSCQVTHEGSTVEKTVAPTECS |
| | | | Acid Sequence | |
| 145 | Human | IGLC6*01 | Cλ Light Chain | ggtcagcccaaggctgccccatcggtcactctgttcccgccctcctctgag |
| | Cλ | | Constant Region | gagcttcaagccaacaaggccacactggtgtgcctgatcagtgacttctac |
| | constant | | (IGLC6*01) Nucleotide | ccgggagctgtgaaagtggcctggaaggcagatggcagccccgtcaaca |
| | region | | Sequence | cgggagtggagaccaccacaccctccaaacagagcaacaacaagtacg |
| | | | | cggccagcagctacctgagcctgacgcctgagcagtggaagtcccacag |
| | | | | aagctacagctgccaggtcacgcatgaagggagcaccgtggagaagac |
| | | | | agtggcccctgcagaatgttca |
| 146 | | | Cλ Light Chain | GQPKAAPSVTLFPPSSEELQANKATLVCLISDFYPGAVKVA |
| | | | Constant Region | WKADGSPVNTGVETTTPSKQSNNKYAASSYLSLTPEQWKS |
| | | | (IGLC6*01) Amino | HRSYSCQVTHEGSTVEKTVAPAECS |
| | | | Acid Sequence | |
| 147 | Human | IGLC7*01 | Cλ Light Chain | ggtcagcccaaggctgccccatcggtcactctgttcccaccctcctctgag |
| | Cλ | or | Constant Region | gagcttcaagccaacaaggccacactggtgtgtctcgtaagtgacttctac |
| | constant | IGLC7*02 | (IGLC7*01 or | ccgggagccgtgacagtggcctggaaggcagatggcagccccgtcaagg |
| | region | | IGLC7*02) Nucleotide | tgggagtggagaccaccaaaccctccaaacaaagcaacaacaagtatgc |
| | | | Sequence | ggccagcagctacctgagcctgacgcccgagcagtggaagtcccacaga |
| | | | | agctacagctgccgggtcacgcatgaagggagcaccgtggagaagaca |
| | | | | gtggcccctgcagaatgctct |
| 148 | | | Cλ Light Chain | GQPKAAPSVTLFPPSSEELQANKATLVCLVSDFYPGAVTVA |
| | | | Constant Region | WKADGSPVKVGVETTKPSKQSNNKYAASSYLSLTPEQWKS |
| | | | (IGLC7*01) Amino | HRSYSCRVTHEGSTVEKTVAPAECS |
| | | | Acid Sequence | |
| 149 | Human | IGLC7*03 | Cλ Light Chain | GGTCAGCCCAAGGCTGCCCCCTCGGTCACTCTGTTCCCA |
| | Cλ | | Constant Region | CCCTCCTCTGAGGAGCTTCAAGCCAACAAGGCCACACTG |
| | constant | | (IGLC7*03) Nucleotide | GTGTGTCTCGTAAGTGACTTCAACCCGGGAGCCGTGACA |
| | region | | Sequence | GTGGCCTGGAAGGCAGATGGCAGCCCCGTCAAGGTGG |
| | | | | GAGTGGAGACCACCAAACCCTCCAAACAAAGCAACAAC |
| | | | | AAGTATGCGGCCAGCAGCTACCTGAGCCTGACGCCCGA |
| | | | | GCAGTGGAAGTCCCACAGAAGCTACAGCTGCCGGGTCA |
| | | | | CGCATGAAGGGAGCACCGTGGAGAAGACAGTGGCCCCT |
| | | | | GCAGAATGCTCT |

TABLE 10
GENE SEGMENT USAGE

| Antibody | IGHV | IGHD | IGHJ | CDRH3 length | IGLV | IGLJ | CDRL3 length |
|---|---|---|---|---|---|---|---|
| ID | gene | gene | gene | (IMGT) | gene | gene | (IMGT) |
| G09 | IGHV3- | IGHD3- | IGHJ6*02 | 20 | IGKV1D- | IGKJ4*01 | 13 |
| | 15*01 | 9*01 | | | 39*01 | | |
| F05 | IGHV3- | IGHD3- | IGHJ5*02 | 18 | IGKV3- | IGKJ4*01 | 9 |
| | 23*04 | 9*01 | | | 11*01 | | |
| C02 | IGHV3- | IGHD3- | IGHJ4*02 | 15 | IGKV1- | IGKJ4*01 | 9 |
| | 23*04 | 10*01 | | | 16*02 | | |
| E04 | IGHV3- | IGH6- | IGHJ4*02 | 11 | IGKV1- | IGKJ1*01 | 9 |
| | 15*01 | 19*01 | | | 5*03 | | |

1. An antibody or fragment comprising a binding site which specifically binds to CD7 (Cluster of Differentiation 7), wherein the binding site comprises a VH domain that is encoded by a nucleotide sequence that is derived from the recombination of a human VH gene segment, DH gene segment and JH gene segment, wherein the VH gene segment is IGHV3-15.

2. The antibody or fragment according to claim 1, wherein the DH gene segment is a human gene segment selected from IGHD3-9 and IGH6-19 and/or wherein the JH gene segment is a human gene segment selected from IGHJ6 and IGHJ4.

3. The antibody or fragment of claim 1 comprising
(a) a VH domain which comprises a CDRH3 sequence of an antibody selected from G09 and E04 or said sequence comprising 3, 2 or 1 amino acid substitution(s); and optionally the VH domain comprises a CDRH1 sequence of said selected antibody or said CDRH1 sequence comprising 3, 2 or 1 amino acid substitution(s); or
(b) a VH domain which comprises a CDRH3 sequence of an antibody selected from G09 and E04 or said CDRH3 sequence comprising 3, 2 or 1 amino acid substitution(s); and optionally comprises a CDRH2 sequence of said selected antibody or said CDRH2 sequence comprising 3, 2 or 1 amino acid substitution(s); or
(c) a VH domain which comprises a CDRH1, 2 and 3 sequence of an antibody selected from G09 and E04 or said sequence comprising 3, 2 or 1 amino acid substitution(s); or
(d) a CDRH3 sequence selected from SEQ ID NO: 3, 6, 63 and 66.

4. The antibody or fragment of claim 1, wherein the binding site comprises a VH domain that comprises the amino acid sequence of a VH domain of an antibody selected from G09 and E04; or an amino acid that is at least 70% identical thereto.

5. (canceled)

6. An antibody or fragment comprising a binding site which specifically binds to CD7, wherein the binding site comprises a VL domain that is encoded by a nucleotide sequence that is derived from the recombination of a human VL gene segment and JL gene segment, wherein the VL gene segment is IGKV1D-39.

7. The antibody or fragment according to claim 6, wherein the VL is a VK and the JL gene segment is a human gene segment IGKJ4.

8. The antibody or fragment of claim 1, comprising
(e) a VL domain which comprises a CDRL3 sequence selected from SEQ ID NO: 13 and 16, or said selected CDRL3 sequence comprising 3, 2 or 1 amino acid substitution(s);
(f) a VL domain which comprises a CDRL3 (and optionally a CDRH3) sequence of antibody selected from G09 E04 or said sequence(s) each comprising 3, 2 or 1 amino acid substitution(s) and optionally the VL domain comprises a CDRL1 sequence of antibody G09 or said CDR1 sequence(s) each comprising 3, 2 or 1 amino acid substitution(s); or
(g) a VL domain which comprises a CDRL3 (and optionally a CDRH3) sequence of antibody selected G09 or said CDR3 sequence(s) each comprising 3, 2 or 1 amino acid substitution(s); and optionally a CDRL2 sequence of antibody G09 or said CDR2 sequence(s) each comprising 3, 2 or 1 amino acid substitution(s).

9. The antibody or fragment according to claim 1 which comprises
(h) the heavy chain amino acid sequence of an antibody selected from G09 and E04; or an amino acid that is at least 70% identical thereto; and/or
(i) the light chain amino acid sequence of an antibody selected from G09 and E04; or an amino acid that is at least 70% identical thereto.

10. The antibody or fragment according to claim 1,
wherein the binding site comprises a VL domain that comprises the amino acid sequence of a VL domain of an antibody selected from G09 and E04; or an amino acid that is at least 70% identical thereto.

11-12. (canceled)

13. The antibody or fragment according to claim 1,
wherein the antibody or fragment comprises a human constant region that comprises the amino acid sequence of SEQ ID NO: 88, 90, 92, 94 or 96 (eg, SEQ ID NO: 88).

14. The antibody or fragment according to claim 1 further comprising an antigen-binding site that specifically binds another target antigen.

15. A method for treating or preventing a CD7-mediated disease or condition in a subject, comprising administering the anti-CD7 antibody or fragment of claim 1 to the subject.

16. (canceled)

17. The method of claim 15, wherein the antibody or fragment is administered to the subject simultaneously or sequentially with chemotherapy or an immune checkpoint inhibitor.

18. A combination comprising the anti-CD7 antibody or fragment of claim 1 and a chemotherapeutic.

19. A method of treating leukaemia in a human comprising administering the anti-CD7 antibody or fragment of claim 1, wherein the antibody, fragment or combination is administered to the human with an antagonist of human CD5, CD14 or CD19.

20-22. (canceled)

23. A pharmaceutical composition comprising an antibody, fragment or combination as defined in claim 1 and a pharmaceutically acceptable excipient, diluent or carrier.

24. A nucleic acid that
(j) encodes a VH domain of an antibody or fragment as defined in claim 1;
(k) encodes (i) a VH domain comprising the amino acid sequence of a VH domain of an antibody selected from G09 and E04; or an amino acid that is at least 70% identical thereto; and/or (ii) a VL domain comprising the amino acid sequence of a VL domain of an antibody selected from G09 and E04; or an amino acid that is at least 70% identical thereto;
(l) comprises a nucleotide sequence that is at least 70% identical to the sequence of SEQ ID NO: 10; and/or a nucleotide sequence that is at least 70% identical to the sequence of SEQ ID NO: 20;
(m) encodes a heavy chain of an antibody or fragment as defined in claim 1;
(n) encodes (i) a heavy chain comprising an amino acid sequence that is at least 70% identical to SEQ ID NO: 7 and/or (ii) a light chain comprising an amino acid sequence that is at least 70% identical to SEQ ID NO: 17; or
(o) comprises a nucleotide sequence that is at least 70% identical to a heavy chain sequence selected of an antibody selected from G09 and E04; and/or a nucleotide sequence that is at least 70% identical to a sequence selected of an antibody selected from G09 and E04.

25. (canceled)

26. A host cell comprising the nucleic of claim 24.

27. A method of diagnosing a CD7-mediated disease or condition in a subject, the method comprising combining the antibody or fragment of claim 1 with an isolated blood or serum sample and determining that cells comprised by the sample are specifically bound by the antibody or fragment.

28. An in vitro assay for detecting CD7-positive cells in a sample, the assay comprising combining antibody or fragment of claim 1 with an isolated blood or serum sample and determining that cells comprised by the sample are specifically bound by the antibody or fragment.
